(19) United States
(12) Patent Application Publication
(10) Pub. No.: US 2026/0049970 A1
(43) Pub. Date: Feb. 19, 2026

(54) ASSET DEVELOPMENT OPTIMIZATION USING FIELD SAMPLING AND ROCK-FLUID INTERACTION TESTING

(71) Applicant: Chevron U.S.A. Inc., San Ramon, CA (US)

(72) Inventors: Wei Wei, Sugar Land, TX (US); David Gilbert Leach, Houston, TX (US); Haiping Lu, Sugar Land, TX (US); Chao Yan, Sugar Land, TX (US); Wei Wang, Houston, TX (US); Edward Russell Peacher, II, Cypress, TX (US); Ramez Hanna Alla, Midland, TX (US); Michael Christopher Cheshire, Richmond, TX (US)

(21) Appl. No.: 19/077,125

(22) Filed: Mar. 12, 2025

Related U.S. Application Data
(60) Provisional application No. 63/684,184, filed on Aug. 16, 2024.

(51) Int. Cl. G01N 33/24; E21B 43/30

(52) U.S. Cl. CPC G01N 33/241; E21B 43/30

(57) ABSTRACT

A method for asset development optimization using a rock sample and rock sample-test fluid interaction testing may include combining the rock sample and a test fluid for a period of time, where the rock sample originates from a portion of a subterranean formation through which a wellbore is drilled. The method may also include obtaining a measurement of a hydrocarbon released from the rock sample-test fluid interaction testing after the period of time. The method may further include generating, using the measurement, a forecast of hydrocarbon production potential for a portion of a subterranean formation from which the rock sample is obtained.

### CROSS-REFERENCE TO RELATED APPLICATIONS

This application claims priority to U.S. Provisional Patent Application Ser. No. 63/684,184 titled “Asset Development Optimization Using Field Sampling And Rock-Fluid Interaction Testing” and filed on Aug. 16, 2024, the entire contents of which are hereby incorporated herein by reference.

### TECHNICAL FIELD

The present application is related to subterranean field operations and, more particularly, to asset development optimization using fluid sampling and rock-fluid interaction testing.

### BACKGROUND

Aqueous fluids are injected into reservoirs during hydraulic fracturing operations to stimulate hydrocarbon release and production through wellbores, such as in tight rock and unconventional (TRU) formations. While significant research has been conducted to understand stimulated/drained rock volume, fluid flow characteristics, geomechanics properties, and acoustic properties, there has been limited work to investigate how the interactions between fluid and rock (e.g., shale for tight formations) affect drilling programs (e.g., landing), well operations (e.g., fracturing operations), production performance, and oil recovery

### SUMMARY

In general, in one aspect, the disclosure relates to a method for asset development optimization using a rock sample and rock sample-test fluid interaction testing. The method may include combining the rock sample and a test fluid for a period of time, where the rock sample originates from a portion of a subterranean formation through which a wellbore is drilled. The method may also include obtaining a measurement of a hydrocarbon released from the rock sample-test fluid interaction testing after the period of time. The method may further include generating, using the measurement, a forecast of hydrocarbon production potential for a portion of a subterranean formation from which the rock sample is obtained.

In another aspect, the disclosure relates to a system for improving production performance of a wellbore using a rock sample and rock sample-test fluid interaction testing. The system may include a fluid source that is configured to provide a test fluid. The system may also include an analytic system that includes a testing apparatus and a controller communicably coupled to the testing apparatus. The testing apparatus of the analytic system may be configured to receive, by the vessel, the rock sample that originates from a portion of a subterranean formation through which the wellbore is drilled; receive, by the vessel, the test fluid from the fluid source; and measure, using the sensor device, a measurement of a hydrocarbon and other gases released from the rock sample-test fluid interaction testing in the vessel. The controller of the analytic system may be configured to facilitate generating, using the measurement, a forecast of hydrocarbon production potential for a portion of a subterranean formation from which the rock sample is obtained.

In yet another aspect, the disclosure relates to a computer-implemented method for improving production performance of a wellbore using a rock sample and rock sample-test fluid interaction testing. The computer-implemented method may include facilitate combining the rock sample and a test fluid for a period of time, wherein the rock sample originates from a portion of a subterranean formation through which the wellbore is drilled. The computer-implemented method may also include facilitate obtaining a measurement of a hydrocarbon released from the rock sample-test fluid interaction testing after the period of time. The computer-implemented method may further include generating, using the measurement, a forecast of hydrocarbon production potential for a portion of a subterranean formation from which the rock sample is obtained.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, the same reference numerals used in different figures may designate like or corresponding but not necessarily identical elements.

FIG. 1 shows a field system in which example embodiments may be used.

FIG. 2 shows another field system in which example embodiments may be used.

FIGS. 3A and 3B show detailed views of the field system of FIG. 1 in which example embodiments may be used.

FIG. 4 shows a diagram of a system for well evaluation using water chemistry analysis according to certain example embodiments.

FIG. 5 shows a system diagram of a controller according to certain example embodiments.

FIG. 6 shows a computing device in accordance with certain example embodiments.

FIG. 7 shows a flowchart of a method for asset development optimization using rock-fluid interaction testing according to certain example embodiments.

FIG. 8 shows part of an analytic system according to certain example embodiments.

FIG. 9 shows part of another analytic system according to certain example embodiments.

FIG. 10 shows part of yet another analytic system according to certain example embodiments.

FIG. 11 shows a graph of mass loss of rock samples according to certain example embodiments.

FIG. 12 shows a graph of a gas analysis resulting from a rock-fluid interaction according to certain example embodiments.

FIG. 13 shows a graph of minerology changes in rock before and after a test fluid interaction according to certain example embodiments.

FIG. 14 shows a graph of mass loss of rock based on test fluid interaction according to certain example embodiments.

FIG. 15 shows a graph plotting measurements taken from rock samples according to certain example embodiments.

FIGS. 16 through 19 show graphs related to an example case study according to certain example embodiments.

FIG. 20 shows a graph of a volume of a recommended operation fluid for multiple wells according to certain example embodiments.

FIG. 21 through 23 show graphs related to another example case study according to certain example embodiments.

FIG. 24 shows a graph of changes in calcium and iron in rock samples over a range of depths according to certain example embodiments.

FIG. 25 shows a graph of changes in manganese and zinc in rock samples over a range of depths according to certain example embodiments.

FIG. 26 shows a graph of calcium to iron ratios for rock samples over a range of depths according to certain example embodiments.

FIG. 27 shows part of another analytic system according to certain example embodiments.

FIG. 28 shows a graph of an index value from headspace gas monitoring according to certain example embodiments.

FIG. 29 shows a table that highlights the capabilities of utilizing an example analytic system over time according to certain example embodiments.

FIG. 30 shows another graph of headspace gas monitoring according to certain example embodiments.

FIG. 31 shows another graph of hydrocarbon production potential from headspace gas monitoring according to certain example embodiments.

FIG. 32 shows part of yet another analytic system according to certain example embodiments.

FIG. 33 shows a table that highlights the capabilities of utilizing an example analytic system over time according to certain example embodiments.

FIGS. 34 and 35 show graphs of hydrocarbon production potential for adjacent wellbores based on testing rock samples using the analytic system of FIG. 32 according to certain example embodiments.

FIG. 36 shows a graph of actual hydrocarbon production for wells in three different areas based on testing rock samples using the analytic system of FIG. 32 according to certain example embodiments.

### DETAILED DESCRIPTION

The example embodiments discussed herein are directed to systems, apparatus, methods, and devices for asset development optimization using fluid sampling and rock-fluid interaction testing. In some cases, use of example embodiments may allow for production performance and/or other forms of field operations that occur at the subsurface (e.g., in a fractured subterranean formation adjacent to a well) to be evaluated and improved, which may lead to additional subterranean resources being extracted from the subsurface and/or increasing the injection capacity and life of a saltwater disposal (SWD) well. As defined herein, an asset may be or include one or more existing wells, one or more new wells, one or more subterranean reservoirs, entire field development, and/or other related subterranean assets of interest. As defined here, potential refers to having or showing the capacity to become or develop into something in the future based on forecasts using example embodiments.

Asset development optimization, as defined herein, may have one or more of a number of results. For instance, example embodiments may be used to optimize (e.g., improve, enhance, increase) asset development by determining where new wells are to be placed and/or landed. In other words, example embodiments may be used to determine the kick-off point (e.g., in terms of depth within the wellbore corresponding to a layer of a subterranean formation) to start a substantially horizontal section of a wellbore from a substantially vertical section of the wellbore (e.g. a new wellbore, an existing wellbore).

As another example, an asset may be optimally developed using example embodiments by determining which wellbores to close off (e.g. safely abandon) and which wellbores (and at what depths within those wellbores) to further develop (e.g., implement fracturing operations) to enhance hydrocarbon production based on the hydrocarbon production potential identified using example embodiments. In some cases, example embodiments may also be used in such cases to provide specific recommendations (e.g., in terms of fracturing fluid composition, in terms of location(s) within a horizontal section of the wellbore) as to how the wellbore may be further developed.

For instance, by changing the test fluid and/or monitoring the gas, liquid, and/or other substances released from the rock samples during the rock-fluid interaction tests discussed herein, the composition of the fluid (e.g., fracturing fluid) used during completion (e.g., hydraulic fracturing) and treatment (e.g., during production stage) of a wellbore to improve hydrocarbon production potential may be optimized. In this way, example embodiments may be used to optimize fracturing fluid composition during initial composition and refracturing, and the hydraulic intervention of the well using the identified optimal fracturing fluid may then release hydrocarbons from the rock in the layer of the subterranean formation in the most economic/effective manner.

In this way, example embodiments may be used to understand the impact of surface area/gram of rock on hydrocarbon recovery efficiency. For instance, test results with 1 gram of rock samples in the form of cuttings with sizes of A, B, and C may be used to compare the gas and/or liquid hydrocarbons released from each size of the rock samples. The results may be used to understand fracture geometry, rock properties, and/or the impact of the generated surface on potential hydrocarbon recovery. This analysis may lead to understanding how to optimize a fracturing program and/or a refracturing program. When strong test fluids such as strong acids are used to interact with rock samples, the results may correlate to the maximum hydrocarbon potential that may be released from the rock, which establishes a baseline for determining recovery efficiency using the hydraulic fracturing approach. The rock-fluid interaction of rock samples according to example embodiments may be used to determine where to drill and/or land a new wellbore.

As yet another example, an asset may be optimally developed using example embodiments by determining how to better deploy capital of a field (e.g., multiple wellbores) or a single wellbore. For instance, if a certain part of the landing or lateral of a wellbore is not known to show hydrocarbon release, but the lateral has been drilled, an operator can make a conscious choice not to stimulate a certain part of the lateral. Example embodiments may additionally or alternatively be used to determine whether further development of an existing well should be paused, reevaluated, altered, or stopped.

Examples of such additional subterranean resources may include, but are not limited to, oil and natural gas. Use of example embodiments on production and injection wells may be designed to comply with certain standards and/or requirements. Example embodiments may be used for wellbores drilled in conventional and/or unconventional (e.g., tight shale) subterranean formations and reservoirs. Example embodiments of asset development optimization (e.g., for injection SWD wells, for production wells) using fluid sampling and rock-fluid interaction testing may be at a subsurface (e.g., within and adjacent to a wellbore in a subterranean formation) for injection (e.g., SWD) wells and production wells (e.g., wells undergoing a fracturing operation).

Example embodiments may relate to a method and workflow to characterize, forecast, and/or improve reservoir and production performance and/or asset development in shale and tight plays using scalable solutions. For example, example embodiments may relate to a new workflow between drilling and completion to improve (e.g., optimize) asset development and production performance. Example embodiments may be designed to investigate the chemical interactions between fluid and shale rock and their indication and applications in reservoir and production performance optimization. Example embodiments may be used for key decision making in unconventional (e.g., shale & tight asset development) formations. Example embodiments may be utilized to understand/forecast GOR and water cut. Example embodiments may be utilized to perform field case root cause analysis/operations troubleshooting in a certain area. Example embodiments may be used to develop and implement a drill-to-fracture strategy that utilizes rock and fluid samples collected during the drilling stage to tailor and optimize a completion strategy and forecast production performance for an individual development well.

Asset development optimization using fluid sampling and rock-fluid interaction testing according to example embodiments may provide results and insights into one or more of a number of factors related to a well. Such factors may include but are not limited to water geochemistry surveillance (e.g., for SWD wells, for a fracture-driven interaction (FDI)), fracturing fluid chemistry and chemical additives, hydrocarbon properties, geoscience considerations (e.g., structural configurations, lithology, stratigraphy methods, gross thickness, net-to-gross ratio, net pay, porosity, saturation, permeability, heterogenicity), engineering considerations (e.g., reservoir depth, pressure, temperature, fluid properties, well placement, landing for a well, recovery mechanisms, fluid mobilities, fluid distribution, well productivity), and/or operational considerations (e.g., water depth, water cut, well types, completion, spacing, facility type and constraints, artificial lift, pattern type and spacing, injector/producer ratio).

As defined herein, a field sample (e.g., a fluid sample, a rock sample) obtained from a well may be or include one or more of any of a number of materials. A field sample (sometimes referred to herein as a rock sample or more simply as a sample) obtained from a well may be or include a liquid, a solid, and/or a gas. In certain example embodiments, a field sample obtained from a well includes some amount (e.g., trace amounts, 5% by volume or weight, 50% by volume or weight, 75% by volume or weight, 95% by volume or weight) of water. Such water may include one or more elements in addition to hydrogen and oxygen. In implementations, a field sample may include produced water, formation water, fracturing fluid (also referred to as fracturing water), and/or hydrocarbon (e.g., oil). A field sample may be from cuttings. In addition, or in the alternative, a field sample may be a core sample.

An FDI may include practically any fluidic interaction involving one or more fractures. For example, the fluidic interaction may be related to fractures generated by hydraulic fracturing. For example, the fluidic interaction may be related to fractures generated from injection, such as saltwater injection. Example embodiments may apply to practically any fluidic interaction, such as those caused by fractures. In one implementation, the fluidic interaction may be between a parent well and a child well. In one implementation, the fluidic interaction may be between a first child well and a second child well.

In one implementation, the fluidic interaction may be between a first formation or first zone and a second formation or second zone that both produce water to a single well, such as a saltwater disposal zone above a hydrocarbon producing zone that both produce water into a single well. In one implementation, the fluidic interaction may be between a first formation or first zone and a second formation or second zone that produce water to a single well, such as a saltwater disposal zone below a hydrocarbon producing zone that both produce water into a single well. In one implementation, the fluidic interaction may be between a first formation or first zone and a second formation or second zone that produce water to a single well, such as a saltwater disposal zone above a hydraulic fracturing zone that both produce water into a single well.

Potentially, in one implementation, the fluidic interaction may be between a first formation or first zone and a second formation or second zone that produce water to a single well, such as a saltwater disposal zone below a hydraulic fracturing zone that both produce water into a single well. This is not an exhaustive list of fluidic interactions, and example embodiments may be applied to other instances of fluidic interaction (e.g., fluidic interactions with three wells, a well in fluidic interaction with three or more formations/three or more zones, multiple wells in fluidic interaction with two or more formations/two or more zones, etc.).

As defined herein, water may be of any type and/or from any source of water, including but not limited to produced water, formation water, without adding any chemicals or making any other alterations to the water. Alternatively, water may be of any type and/or from any source of water that has added thereto one or more chemicals and/or has otherwise been altered in some way. Examples of such water may include, but are not limited to, water within a fracturing fluid, water with an acid added to it, and water with scale inhibitor added to it. The water may include one or more types of solid-generating components (e.g., bivalent cations, trivalent cations). In addition, the water may include various amounts of total dissolved solids (TDSs) (e.g., between 1,000 mg/L and 500,000 mg/L, between 30,000 mg/L and 100,000 mg/L, between 50,000 mg/L and 250,000 mg/L, between 20,000 mg/L and 50,000 mg/L, between 100,000 mg/L and 200,000 mg/L).

The use of the terms “about”, “approximately”, and similar terms applies to all numeric values, whether or not explicitly indicated. These terms generally refer to a range of numbers that one of ordinary skill in the art would consider as a reasonable amount of deviation to the recited numeric values (i.e., having the equivalent function or result). For example, this term may be construed as including a deviation of ±10 percent of the given numeric value provided such a deviation does not alter the end function or result of the value. Therefore, a value of about 1% may be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein.

A “subterranean formation” refers to practically any volume under a surface. For example, it may be practically any volume under a terrestrial surface (e.g., a land surface), practically any volume under a seafloor, etc. Each subsurface volume of interest may have a variety of characteristics, such as petrophysical rock properties, reservoir fluid properties, reservoir conditions, hydrocarbon properties, or any combination thereof. For example, each subsurface volume of interest may be associated with one or more of: temperature, porosity, salinity, permeability, water composition, mineralogy, hydrocarbon type, hydrocarbon quantity, reservoir location, pressure, etc. Those of ordinary skill in the art will appreciate that the characteristics are many, including, but not limited to: shale gas, shale oil, tight gas, tight oil, tight carbonate, carbonate, vuggy carbonate, unconventional formation (e.g., a permeability of less than 25 millidarcy (mD) such as a permeability of from 0.000001 mD to 25 mD)), diatomite, geothermal, mineral, etc.

In some embodiments, an unconventional formation may have a permeability of less than 25 millidarcy (mD) (e.g., 20 mD or less, 15 mD or less, 10 mD or less, 5 mD or less, 1 mD or less, 0.5 mD or less, 0.1 mD or less, 0.05 mD or less, 0.01 mD or less, 0.005 mD or less, 0.001 mD or less, 0.0005 mD or less, 0.0001 mD or less, 0.00005 mD or less, 0.00001 mD or less, 0.000005 mD or less, 0.000001 mD or less, or less). In some embodiments, an unconventional formation may have a permeability of at least 0.000001 mD (e.g., at least 0.000005 mD, at least 0.00001 mD, 0.00005 mD, at least 0.0001 mD, 0.0005 mD, 0.001 mD, at least 0.005 mD, at least 0.01 mD, at least 0.05 mD, at least 0.1 mD, at least 0.5 mD, at least 1 mD, at least 5 mD, at least 10 mD, at least 15 mD, or at least 20 mD).

An unconventional formation may include a permeability ranging from any of the minimum values described above to any of the maximum values described above. For example, in some embodiments, the unconventional formation may have a permeability of from 0.000001 mD to 25 mD (e.g., from 0.001 mD to 25 mD, from 0.001 mD to 10 mD, from 0.01 mD to 10 mD, from 0.1 mD to 10 mD, from 0.001 mD to 5 mD, from 0.01 mD to 5 mD, or from 0.1 mD to 5 mD).

The terms “formation”, “subsurface formation”, “hydrocarbon-bearing formation”, “reservoir”, “subsurface reservoir”, “subsurface area of interest”, “subsurface region of interest”, “subsurface volume of interest”, and the like may be used synonymously. The term “subterranean formation” is not limited to any description or configuration described herein.

A “well” or a “wellbore” refers to a single hole, usually cylindrical, that is drilled into a subsurface volume of interest. A well or a wellbore may be drilled in one or more directions. For example, a well or a wellbore may include a vertical well, a horizontal well, a deviated well, and/or other type of well. A well or a wellbore may be drilled in the subterranean formation for exploration and/or recovery of resources. A plurality of wells (e.g., tens to hundreds of wells) or a plurality of wellbores are often used in a field depending on the desired outcome.

A well or a wellbore may be drilled into a subsurface volume of interest using practically any drilling technique and equipment known in the art, such as geosteering, directional drilling, etc. Drilling the well may include using a tool, such as a drilling tool that includes a drill bit and a drill string. Drilling fluid, such as drilling mud, may be used while drilling in order to cool the drill tool and remove cuttings. Other tools may also be used while drilling or after drilling, such as measurement-while-drilling (MWD) tools, seismic-while-drilling tools, wireline tools, logging-while-drilling (LWD) tools, or other downhole tools. After drilling to a predetermined depth, the drill string and the drill bit may be removed, and then the casing, the tubing, and/or other equipment may be installed according to the design of the well. The equipment to be used in drilling the well may be dependent on the design of the well, the subterranean formation, the hydrocarbons, and/or other factors.

A well may include a plurality of components, such as, but not limited to, a casing, a liner, a tubing string, a sensor, a packer, a screen, a gravel pack, artificial lift equipment (e.g., an electric submersible pump (ESP)), and/or other components. If a well is drilled offshore, the well may include one or more of the previous components plus other offshore components, such as a riser. A well may also include equipment to control fluid flow into the well, control fluid flow out of the well, or any combination thereof. For example, a well may include a wellhead, a choke, a valve, and/or other control devices. These control devices may be located on the surface, in the subsurface (e.g., downhole in the well), or any combination thereof.

In some embodiments, the same control devices may be used to control fluid flow into and out of the well. In some embodiments, different control devices may be used to control fluid flow into and out of a well. In some embodiments, the rate of flow of fluids through the well may depend on the fluid handling capacities of the surface facility that is in fluidic communication with the well. The equipment to be used in controlling fluid flow into and out of a well may be dependent on the well, the subsurface region, the surface facility, and/or other factors. Moreover, sand control equipment and/or sand monitoring equipment may also be installed (e.g., downhole and/or on the surface). A well may also include any completion hardware that is not discussed separately. The term “well” may be used synonymously with the terms “borehole,” “wellbore,” or “well bore.” The term “well” is not limited to any description or configuration described herein.

“Hydraulic fracturing” is one way that hydrocarbons may be recovered (sometimes referred to as produced) from the formation. For example, hydraulic fracturing may entail preparing a fracturing fluid and injecting that fracturing fluid into the wellbore at a sufficient rate and pressure to open existing fractures and/or create fractures in the formation. The fractures permit hydrocarbons to flow more freely into the wellbore. In the hydraulic fracturing process, the fracturing fluid may be prepared on-site to include at least proppants. The proppants, such as sand or other particles, are meant to hold the fractures open so that hydrocarbons may more easily flow to the wellbore. The fracturing fluid and the proppants may be blended together using at least one blender. The fracturing fluid may also include other components in addition to the proppants.

The wellbore and the formation proximate to the wellbore are in fluid communication (e.g., via perforations), and the fracturing fluid with the proppants is injected into the wellbore through a wellhead of the wellbore using at least one pump (oftentimes called a fracturing pump). The fracturing fluid with the proppants is injected at a sufficient rate and pressure to open existing fractures and/or create fractures in the subsurface volume of interest. As fractures become sufficiently wide to allow proppants to flow into those fractures, proppants in the fracturing fluid are deposited in those fractures during injection of the fracturing fluid. After the hydraulic fracturing process is completed, the fracturing fluid is removed by flowing or pumping it back out of the wellbore so that the fracturing fluid does not block the flow of hydrocarbons to the wellbore. The hydrocarbons will typically enter the same wellbore from the formation and go up to the surface for further processing.

The equipment to be used in preparing and injecting the fracturing fluid may be dependent on the components of the fracturing fluid, the proppants, the wellbore, the formation, etc. However, for simplicity, the term “fracturing apparatus” is meant to represent any tank(s), mixer(s), blender(s), pump(s), manifold(s), line(s), valve(s), fluid(s), fracturing fluid component(s), proppants, and other equipment and non-equipment items related to preparing the fracturing fluid and injecting the fracturing fluid.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A.

In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C.

In some embodiments, the item described by this phrase could include two or more components of type A (e.g., A1 and A2). In some embodiments, the item described by this phrase could include two or more components of type B (e.g., B1 and B2). In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C).

In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure may be inferred to that component. Conversely, if a component in a figure is labeled but is not described, the description for such component may be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of asset development optimization using fluid sampling and rock-fluid interaction testing will be described more fully hereinafter with reference to the accompanying drawings, in which example embodiments of asset development optimization using fluid sampling and rock-fluid interaction testing are shown. Asset development optimization using fluid sampling and rock-fluid interaction testing may, however, be embodied in many different forms and should not be construed as limited to the example embodiments set forth herein. Rather, these example embodiments are provided so that this disclosure will be thorough and complete, and will fully convey the scope of asset development optimization using fluid sampling and rock-fluid interaction testing to those of ordinary skill in the art. Like, but not necessarily the same, elements (also sometimes called components) in the various figures are denoted by like reference numerals for consistency.

Terms such as “first”, “second”, “primary,” “secondary,” “above”, “below”, “inner”, “outer”, “distal”, “proximal”, “end”, “top”, “bottom”, “upper”, “lower”, “side”, “left”, “right”, “front”, “rear”, and “within”, when present, are used merely to distinguish one component (or part of a component or state of a component) from another. This list of terms is not exclusive. Such terms are not meant to denote a preference or a particular orientation, and they are not meant to limit embodiments of asset development optimization using fluid sampling and rock-fluid interaction testing. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

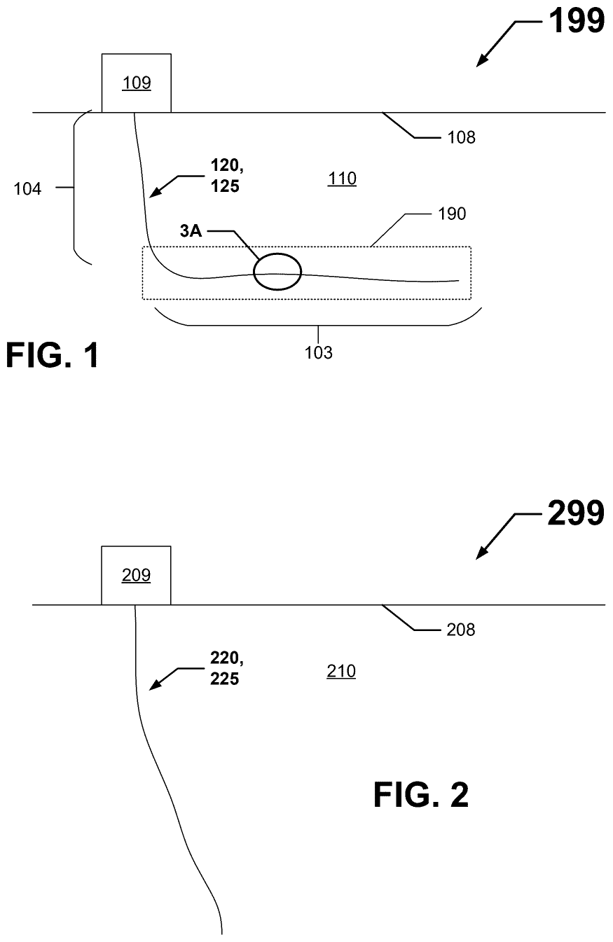

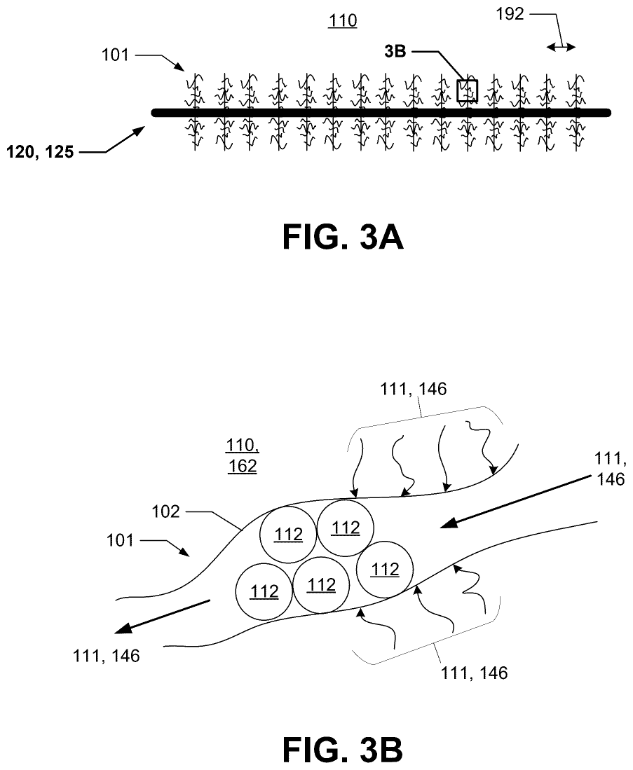

FIG. 1 shows a schematic diagram of a land-based field system 199 with which example embodiments may be used. FIG. 2 shows a schematic diagram of another land-based field system 299 with which example embodiments may be used. FIG. 3A shows a detail of a substantially horizontal section 103 of the wellbore 120 of FIG. 1. FIG. 3B shows a detail of a fracture 101 of FIG. 3A. The field system 199 of FIG. 1 includes a producing wellbore 120 disposed in a subterranean formation 110 using field equipment 109 (e.g., a derrick, a tool pusher, a clamp, a tong, drill pipe, casing pipe, a drill bit, a wireline tool, a fluid pumping system) located above a surface 108 and within the wellbore 120. Example embodiments may also be used in other types of wells (e.g., injection wells) that have vertical sections 104 and/or horizontal sections 103.

With respect to the system 199 of FIG. 1, once the wellbore 120 is drilled, a casing string 125 is inserted into the wellbore 120 to stabilize the wellbore 120 and allow for the extraction of subterranean resources (e.g., natural gas, oil, produced water) from the subterranean formation 110. Field equipment 109, located at the surface 108, is used to drill, encase, fracture, produce, and/or perform any other part of a field operation with respect to the wellbore 120. The wellbore 120 of FIG. 1 starts out with a substantially vertical section 104 (e.g., no more than 45° from true vertical, no more than 30° from true vertical), and then has a substantially horizontal section 103 (e.g., no more than 45° from true horizontal, no more than 30° from true horizontal). This configuration of the wellbore 120 is common for exploration and production of subterranean resources, such as oil and natural gas.

Similarly, with respect to the system 299 of FIG. 2, once the wellbore 220 is drilled, a casing string 225 is inserted into the wellbore 220 to stabilize the wellbore 220 from the subterranean formation 210. Field equipment 209, located at the surface 208, is used to drill, encase, fracture, produce, and/or perform any other part of a field operation with respect to the wellbore 220. The wellbore 220 of FIG. 2 is substantially vertical. This configuration of the wellbore 220 is common for injection wells.

Referring back to FIG. 1, the surface 108 may be ground level for an onshore application and the sea floor (or other similar floor under a body of water) for an offshore application. A body of water may include, but it not limited to, sea water, brackish water, flowback or produced water, wastewater (e.g., reclaimed or recycled), brine (e.g., reservoir or synthetic brine), fresh water (e.g., fresh water comprises <1,000 ppm TDS), any other type of water, or any combination thereof. For offshore applications, at least some of the field equipment may be located on a platform that sits above the water level. The point where the wellbore 120 begins at the surface 108 may be called the wellhead.

While not shown in FIGS. 1 and 2, there may be multiple wellbores 120, 220, each with its own wellhead but that is located close to the other wellheads, drilled into the subterranean formation 110, 210 and having substantially vertical sections and/or horizontal sections 103 that are close to each other. In such a case, the multiple wellbores 120, 220 may be drilled at the same pad or at different pads.

During the process of drilling the wellbore 120 of FIG. 1, as detailed in FIGS. 3A and 3B, cuttings, water 146 (e.g., produced water, formation water), and other subterranean resources 111 (e.g., relatively small amounts of oil or natural gas) may be extracted (or otherwise obtained) from downhole to the surface 108, where some of the field equipment 109 separates out at least some of the cuttings and recirculates the produced water back downhole. When the drilling process is complete, other operations, such as fracturing operations, may be performed. While the subterranean formation 110 may have naturally-occurring fractures 101 and some fractures 101 that may be created when drilling the wellbore 120, these fractures 101 may need to be enlarged and/or elongated, and additional fractures 101 may need to be created, in order to extract additional subterranean resources 111 (e.g., oil, natural gas) from the subsurface. The fractures 101 are shown to be located in the horizontal section 103 of the wellbore 120 in FIG. 1. The fractures 101, whether created and/or naturally occurring, may additionally or alternatively be located in other sections (e.g., a substantially vertical section 104, a transition area between a vertical section 104 and a horizontal section 103) of the wellbore 120. In some cases, a wellbore 220 has no substantially horizontal sections, as shown in FIG. 2. Example embodiments may be used along any portion of a wellbore (e.g., wellbore 120, wellbore 220), regardless of whether fractures 101 are located in such portion.

The subterranean formation 110 may include one or more of a number of formation types, including but not limited to shale, limestone, sandstone, clay, sand, and salt. In certain embodiments, a subterranean formation 110 may include one or more reservoirs in which one or more subterranean resources 111 (e.g., oil, natural gas, water, steam) may be located. One or more of a number of field operations (e.g., fracturing (e.g., hydraulic fracturing), coring, tripping, drilling, setting casing, extracting downhole resources, production) may be performed to reach an objective of a user with respect to the subterranean formation 110.

The wellbore 120 may have one or more of a number of segments or hole sections, where each segment or hole section may have one or more of a number of dimensions. Examples of such dimensions may include, but are not limited to, a size (e.g., diameter) of the wellbore 120, a curvature of the wellbore 120, a true vertical depth of the wellbore 120, a measured depth of the wellbore 120, and a horizontal displacement of the wellbore 120. There may be multiple overlapping casing strings of various sizes (e.g., length, outer diameter) contained within and between these segments or hole sections to ensure the integrity of the wellbore construction. In this case, one or more of the segments of the subterranean wellbore 120 is the substantially horizontal section 103.

As discussed above, inserted into and disposed within the wellbore 120 of FIGS. 1 and 2 are a number of casing pipes that are coupled to each other end-to-end to form the casing string 125 and the casing string 225, respectively. In these cases, each end of a casing pipe has mating threads (a type of coupling feature) disposed thereon, allowing a casing pipe to be directly or indirectly mechanically coupled to another casing pipe in an end-to-end configuration. The casing pipes of the casing string 125 and the casing string 225 may be indirectly mechanically coupled to each other using a coupling device, such as a coupling sleeve.

Each casing pipe of the casing string 125 and the casing string 225 may have a length and a width (e.g., outer diameter). The length of a casing pipe may vary. For example, a common length of a casing pipe is approximately 40 feet. The length of a casing pipe may be longer (e.g., 60 feet) or shorter (e.g., 10 feet) than 40 feet. The width of a casing pipe may also vary and may depend on the cross-sectional shape of the casing pipe. For example, when the shape of the casing pipe is cylindrical, the width may refer to an outer diameter, an inner diameter, or some other form of measurement of the casing pipe. Examples of a width in terms of an outer diameter may include, but are not limited to, 4½ inches, 7 inches, 7⅝ inches, 8⅝ inches, 10¾ inches, 13⅜ inches, and 14 inches.

The size (e.g., width, length) of the casing string 125 and the casing string 225 may be based on the information (e.g., diameter of the borehole drilled) gathered using field equipment with respect to the subterranean wellbore 120 and the subterranean wellbore 220, respectively. The walls of the casing string 125 and the casing string 225 have an inner surface that forms a cavity that traverses the length of the casing string 125 and the casing string 225. Each casing pipe may be made of one or more of a number of suitable materials, including but not limited to steel.

Once the wellbore 120, 220 (or a section thereof) is drilled, the casing string 125, 225 is inserted into the wellbore 120, 220 and subsequently cemented to the wellbore 120, 220 to stabilize the wellbore 120, 220 and allow for the extraction of subterranean resources 111 (e.g., oil, natural gas) from the subterranean formation 110. For example, cement may be poured into the wellbore 120 through the cavity and then forced upward between the outer surface of the casing string 125 and the wall of the subterranean wellbore 120. Similarly, cement may be poured into the wellbore 220 through the cavity and then forced upward between the outer surface of the casing string 225 and the wall of the subterranean wellbore 220. In some cases, a liner may additionally be used with, or alternatively be used in place of, some or all of the casing pipes.

Referring to the system 199 of FIGS. 1, 3A, and 3B, once the cement dries, a number of fractures 101 may be created in the subterranean formation 110. The fractures 101 may be created in any of a number of ways known in the industry, including but not limited to hydraulic fracturing, fracturing using electrodes, and/or other methods of generating fractures. The hydraulic fracturing process involves the injection of large quantities of fluids containing water, chemical additives, and proppant 112 into the subterranean formation 110 from the wellbore 120 to create fracture networks. A subterranean formation 110 naturally has fractures 101, but these naturally occurring fractures 101 have inconsistent characteristics (e.g., length, spacing) and so in some cases cannot be relied upon for extracting subterranean resources without having additional fractures 101, such as what is shown in FIG. 3A, created in the subterranean formation 110.

Operations that create fractures 101 in the subterranean formation 110 use any of a number of fluids that include proppant 112 (e.g., sand, ceramic pellets). When proppant 112 is used, some of the fractures 101 (also sometimes called principal or primary fractures) receive proppant 112, while a remainder of the fractures 101 (also sometimes called secondary fractures) do not have any proppant 112 (or small amounts of proppant 112) in them. During a fracturing operation, large quantities of fluid (e.g., water/aqueous based chemicals) are injected into certain locations in the well 120. The hydraulic fracturing process involves the opening of pores (e.g., nanometer-sized fractures 101) in certain formations (e.g., shale) via rock-fluid interaction.

There may be one or more impacts from and/or indications of rock-fluid interactions in the fractures 101. For example, an impact of rock-fluid interactions may be to clear debris from the fractures 101 (such as during an acid stage of a fracturing operation) to better access the subterranean resources 111 within the subterranean formation 110. As another example, an impact of rock-fluid interactions may be to enhance or begin the imbibition process where subterranean resources 111 (e.g., hydrocarbons) are released from the rock. As yet another example, an indication of rock-fluid interactions may be certain produced fluid compositions and chemistry characteristics, which may lead to an indication of certain fluid chemistry related risks (e.g., scale, corrosion, reservoir souring). Using example embodiments, rock-fluid interaction tests may provide important information to characterize, forecast, and/or improve reservoir and well performance for tight rock and unconventional plays.

As shown in FIG. 3B, the proppant 112 is designed to become lodged inside at least some of the created fractures 101 to keep those fractures 101 open after the fracturing operation is complete. The size of the proppant 112 is a design consideration determined by a user. Sizes (e.g., 40/70 mesh, 50/140 mesh) of the proppant 112 may vary. While the shape of the proppant 112 is shown as being uniformly spherical, and the size is substantially identical among the proppant 112, the actual sizes and shapes of the proppant 112 may vary, whether in absolute terms and/or with respect to each other. If the proppant 112 is too small, the proppant 112 will not be effective at keeping the fractures 101 open enough to effectively allow water 146 and/or other subterranean resources 111 to flow through the fractures 101 from the rock matrices 162 in the subterranean formation 110 to the wellbore 120. If the proppant 112 is too large, the proppant 112 may plug up the fractures 101, blocking the flow of the water 146 and/or other subterranean resources 111 through the fractures 101.

The use of proppant 112 in certain types of subterranean formation 110, such as shale, may be important. Shale formations typically have permeabilities on the order of microdarcys (μD) to nanodarcys (nD). When fractures 101 are created in such formations with low permeabilities, it is important to sustain the fractures 101 and their permeability and conductivity for an extended period of time in order to extract more of the subterranean resource 111. Example embodiments may also be applied to fluids used in other types of field operations, including but not limited to fracturing operations and injection wells.

Regardless of the type (e.g., conventional, unconventional) of subterranean formation 110, when proppant 112 and/or other similar components of a fracturing fluid are used in a fracturing operation, The proppant 112 and/or other similar components may be designed to become lodged within fractures 101 that result in principal fractures, which are designed to last (stay open) for a longer period of time as fluids (e.g., water 146, subterranean resources 111) flow therethrough. Fractures 101 that do not have proppant 112 and/or other similar components lodged therein may be referred to as secondary fractures, which may not last as long (close or reduce in size more quickly) as principal fractures.

The various created fractures 101 that originate at the wellbore 120 and extend outward into the rock matrices 162 in the subterranean formation 110 in this case have consistent penetration lengths perpendicular to the wellbore 120 and have consistent coverage along at least a portion of the lateral length (substantially horizontal section) of the wellbore 120. For example, created fractures 101 may be 50 meters high and 200 meters long. Further, the created fractures 101 may be spaced a distance 192 apart from each other. The distance 192 (e.g., 25 meters, 5 meters, 12 meters) may be optimized based on the permeability and/or the porosity of the rock matrix 162 of the subterranean formation 110.

The created fractures 101 create a volume 190 within the subterranean formation 110 where the rock matrix 162 of the subterranean formation 110 is connected to the high conductivity fractures 101 located a short distance away. In addition to different configurations of the fractures 101, other factors that may contribute to the viability of the subterranean formation 110 may include, but are not limited to, permeability of the rock matrix 162, capillary pressure, and the temperature and pressure of the subterranean formation 110. Each fracture 101, whether created or naturally occurring, is defined by a wall 102, also called a fracture face 102 herein. The fracture face 102 provides a transition between the paths formed by the rock matrices 162 in the subterranean formation 110 and the fracture 101. The subterranean resources 111 flow through the paths formed by the rock matrices 162 in the subterranean formation 110 into the fracture 101.

The rock matrices 162, as well as the rest of the subterranean formation 110, both without and outside the volume 190, have a certain amount of water 146 therein. The water 146 may be or include, for example, formation water from the formation matrix within the volume 190, moveable free formation water, and “external” water from non-targeted formation/sources (e.g., outside the target volume 190). These external sources of water 146 may include water from a nearby SWD source(s), a nearby hydrocarbon producing source, and/or other sources.

The water 146 may have any of a number of different components (e.g., minerals, chemical additives, acids, completion brine) in addition to formation water. The contents of water 146 in one part (e.g., outside the volume 190) of the subterranean formation 110 may be the same as, or different than, the contents of the water 146 in other parts (e.g., in the rock matrices 162) of the subterranean formation 110. In some cases, such as during a stage (e.g., a hydraulic fracturing stage) of a field operation, the fluids (e.g., fracturing fluid) used in that stage may mix with or include the water 146, thereby changing the contents or composition of the in situ water chemistry in parts (e.g., at or near the fractures 101) of the subterranean formation 110. The water 146 may include one or more of a number of types of water, including but not limited to sea water, brackish water, flowback or produced water, wastewater (e.g., reclaimed or recycled), brine (e.g., reservoir or synthetic brine), fresh water (e.g., fresh water comprises <1,000 ppm TDS), any other type of water, or any combination thereof.

There may be high levels of lateral and/or horizontal heterogeneity in unconventional formations (e.g., shale). The type curve and/or production forecast based on a pilot study or analog wells may not appropriately represent individual development wells in a certain area. The lack of understanding in the variation of hydrocarbon production potential and water cut along the lateral provide a motivation for using example embodiments. In addition, with unconventional formations, there may be a potential invasion of extraneous movable water sources from non-target formations and/or salt water disposal wells. Water invasion may potentially jeopardize hydrocarbon production from a well 120. In certain cases, a well 120 may completely or substantially lose oil production due to external water invasion with an up to 90% decrease in EUR. Use of example embodiments may help recognize these situations ahead of time and provide a path for producing the well 120 by introducing a particular fluid that interacts with the rock in such a way as to reduce or prevent the external water invasion.

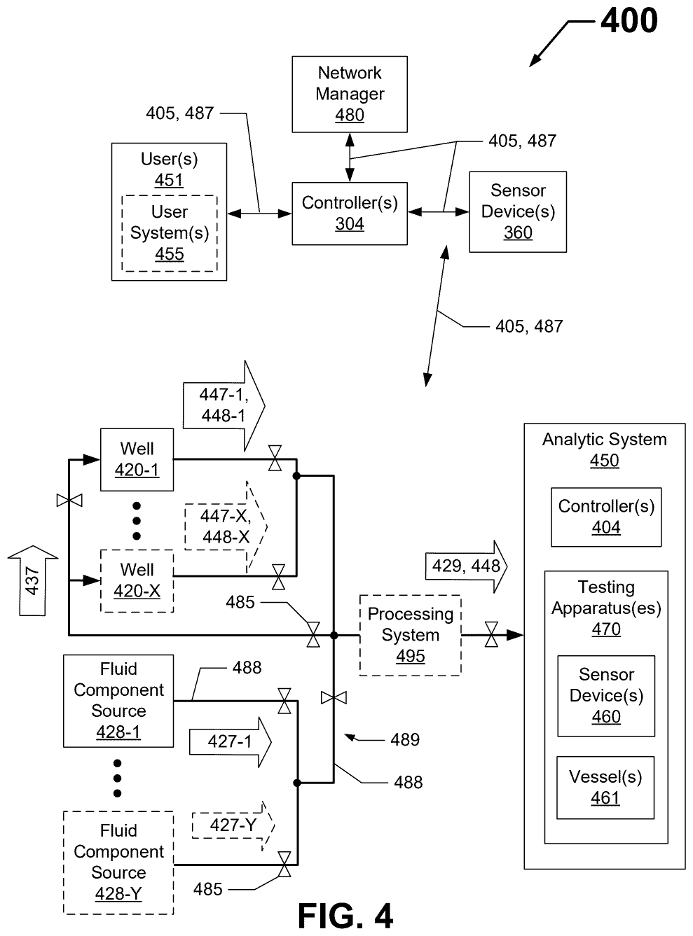

FIG. 4 shows a diagram of a system 400 for well evaluation using water chemistry analysis according to certain example embodiments. The system 400 of FIG. 4 includes one or more fluid component sources 428, one or more wells 420, an example analytic system 450, an optional processing system 495, one or more controllers 304, one or more sensor devices 360, one or more users 451 (including one or more optional user systems 455), a network manager 480, a material conveyance system 488, and one or more valves 485. The analytic system 450 in this case includes one or more testing apparatuses 470 and one or more controllers 404. Each testing apparatus 470 may include one or more vessels 461 and one or more sensor devices 460.

The components shown in FIG. 4 are not exhaustive, and in some embodiments, one or more of the components shown in FIG. 4 may not be included in the example system 400. Any component of the system 400 may be discrete or combined with one or more other components of the system 400. Also, one or more components of the system 400 may have different configurations. For example, one or more sensor devices 360 may be disposed within or disposed on other components (e.g., the material conveyance system 488, a valve 485, a fluid component source 428, a well 420). As another example, a controller 304, rather than being a stand-alone device, may be part of one or more other components (e.g., a fluid component source 428, a well 420) of the system 400.

Incorporating the description above with respect to FIGS. 1 through 3, the system 400 of FIG. 4 may include one or more wells 420 (in this case, well 420-1 through well 420-X). Each of the wells 420 of the system 400 may be substantially similar to the wells discussed above. Some or all of the wells 420 may be from a common pad. Each well 420 may produce water (e.g., water 146), subterranean resources (e.g., subterranean resources 111), cuttings, other materials, or any combination thereof. From these materials that flow uphole from each well 420 to the surface, one or more samples 447 may be obtained. Each sample 447 that is obtained may be transported through the material conveyance system 488, through the optional processing system 495, and to the analytic system 450. Over time, a well 420 may be used for different purposes. For example, well 420-1 may be used as a production well at one time, and at another time, well 420-1 may be used as an injection well.

A sample 447 of FIG. 4 may include rock 448 (e.g., cuttings, core samples). In some cases, a sample 447 may also include water, which may be substantially the same as the water 146 discussed above. Specifically, the water of a sample 447 may be any type of water, including but not limited to the produced water, sea water, brackish water, wastewater (e.g., reclaimed or recycled), brine (e.g., reservoir or synthetic brine), fresh water (e.g., fresh water comprises <1,000 ppm TDS), or any other type of water. In addition to rock 448 (also sometimes referred to herein as rock samples 448) and optionally water, a sample 447 may include fracturing fluid (fracturing water), one or more subterranean resources 111 (e.g., a hydrocarbon (e.g., oil, natural gas)), and/or any other element or compound. In addition to a solid, a sample 447 may be in the form of a liquid and/or a gas. Each sample 447 is specifically categorized as being from a particular well 420. For example, samples 447-1, including rocks 448-1, are from well 420-1, and samples 447-X, including rocks 447-X, are from well 420-X.

The samples 447, including the rock 448, are moved from each well 420 toward the analytic system 450 using a conveyance system 488. The conveyance system 488 may be configured to extract the samples 447, including the rock 448, from a well 420 and/or convey the samples 447, including the rock 448, through the conveyance system 488 toward the analytic system 450. The conveyance system 488 may additionally or alternatively be configured to extract a fluid component 427 from a fluid component source 428 and convey the fluid component 427 through the conveyance system 488 to the analytic system 450. The conveyance system 488 may additionally or alternatively be configured to convey one or more fluid components 427 and/or one or more samples 447 from one or more fluid component sources 428 and/or one or more wells 420, respectively, to the optional processing system 495. The conveyance system 488 may additionally or alternatively be configured to convey one or more processed fluid components 427 and/or one or more processed samples 447 from the optional processing system 495 to the analytic system 450.

As discussed above, the system 400 may also include one or more fluid component sources 428. Each fluid component source 428 may hold one or more fluid components 427. A fluid component source 428 may include, but is not limited to, a natural vessel (e.g., land that forms walls to contain a liquid, a subterranean cavity that holds carbon dioxide or other gas or liquid) and a man-made storage tank or other type of vessel. Each fluid component 427 may be or include a liquid, a solid, and/or a gas. A fluid component 427 may be in the form of a liquid, a gas, and/or a solid. A single fluid component 427 or a mixture of multiple fluid components 427 may be disposed in a fluid component source 428.

Examples of a fluid component 427 may include, but are not limited to, carbon dioxide, gas with various concentrations of CO₂ (e.g., in liquid form, in gas form, in produced gas from a field operation, from a source external to a field operation), hydrocarbons, a chemical used for a fracturing operation, water that does not come from a well 420, methane, H₂S, nucleation catalyzing metals, an alkali salt (e.g., NaOH), sodium bicarbonate (NaHCO₃), sodium carbonate (Na₂CO₃), polymers and/or other substances, and flocculation agents. In some cases, multiple fluid components 427 may be combined to form a fluid 437 (also sometimes called an operational fluid 437 herein). In some other cases, a single fluid component 427 may also be a fluid 437.

A fluid component 427 may serve one or more purposes in one or more field operations. For example, a fluid component 427 may be used in a fluid 437 to generate and/or enhance fractures 101 in a subterranean formation 110 adjacent to a well 420 during a fracturing operation. As another example, a fluid component 427 may be carbon dioxide, a gas stream containing carbon dioxide (e.g., stored, produced), or any combination thereof, which may be used during injection of a well 420 that is an injection well. One of ordinary skill in the art will appreciate that other fluid components 427 and/or combinations thereof are possible in example embodiments.

The conveyance system 488 may include one or more of a number of pieces of equipment to perform its function. Examples of such equipment may include, but are not limited to, a compressor, a motor, a pump, a conveyer, a truck or other vehicle, a rail system, a crane, a shaker, a vibrator, piping, a fan, a blower, a valve (e.g., valve 485), a controller (e.g., controller 404), and a sensor device (e.g., sensor device 460). Some or all of the conveyance system 488 may operate using a controller (e.g., controller 404). In addition, or in the alternative, one or more users 451 may perform one or more of the various functions required to move some or all of the samples 447, one or more of the fluid components 427, and/or one or more of the fluids 437 using the conveyance system 488. The conveyance system 488 (including the collection area 489) may include any components, devices, subsystems, etc. that transport the samples 447, the fluid components 427, and the fluid 437 within the system 400 from one component to another component. The conveyance system 488 may be configured to transport solids, liquids, and/or gases.

For example, in order to transport liquids and gases within the system 400, the conveyance system 488 may include piping. In such a case, the piping of the conveyance system 488 may include multiple pipes, ducts, elbows, joints, sleeves, collars, and similar components that are coupled to each other (e.g., using coupling features such as mating threads) to establish a network for transporting such liquids and/or gases within the system 400. Each component of the piping of the conveyance system 488 may have an appropriate size (e.g., inner diameter, outer diameter) and be made of an appropriate material (e.g., steel, PVC) to safely and efficiently handle the pressure, temperature, flow rate, and other characteristics of the liquids and/or gases that flow therethrough. As another example, in order to transport solids within the system 400, the conveyance system 488 may include conveyer belts, trucks, bulldozers, backhoes, and/or other similar equipment.

There may be a number of valves 485 placed directly or indirectly in-line with the conveyance system 488 (or portions thereof) at various locations in the system 400 to control the flow of the samples 447, the fluid components 427, and/or the fluids 437 in liquid and/or gas form. A valve 485 may have one or more of any of a number of configurations, including but not limited to a guillotine valve, a ball valve, a gate valve, a butterfly valve, a pinch valve, a needle valve, a plug valve, a diaphragm valve, and a globe valve. One valve 485 may be configured the same as or differently compared to another valve 485 in the system 400. Also, one valve 485 may be controlled (e.g., manually by a user 451, automatically by a controller 404 of the analytic system 450, automatically by a controller 304) the same as or differently compared to another valve 485 in the system 400.

In some cases, positioned within the material conveyance system 488 between the wells 420, the fluid component sources 428, and the analytic system 450 may be an optional processing system 495. Such a processing system 495 may be or include part of the field equipment (e.g., field equipment 109, field equipment 209) discussed above. The processing system 495 may be designed to separate cuttings, other subterranean resources 111 (e.g., oil, natural gas), and/or other elements from the samples 447 as the samples 447 are prepared for testing in the analytic system 450 and/or for recirculation into a well 420 (e.g., the same well 420 from which the samples 447 are obtained, another well 420 (e.g., a SWD well)). In addition, or in the alternative, the processing system 495 may be configured to otherwise process (e.g., mix, heat, dry, cool, dehumidify, hydrate, stimulate, agitate, separate) some or all of one or more samples 447, some or all of one or more fluid components 427, and/or some or all of one or more fluids 437 at a point in time and/or over a period of time.

Such a processing system 495 may include one or more of a number of various pieces of equipment. Such equipment may include, but is not limited to, a pump, a motor, a filter, a centrifuge, a heater, a blower, a condenser, a vessel, a funnel, a strainer, a separator, an agitator, a paddle, a circulating system, an aerator, a heat exchanger, a column, a test tube, a separator, a mixer (e.g., a centrifuge mixer, a desander, a tumbler mixer, a homogenizer, a static mixer, a drum mixer, a fluidization mixer, agitator mixers, paddle mixers, an emulsifier, a drum mixer, a pail mixer, a convective mixer, an agitator, a batch mixer, and a ribbon mixer), a controller (e.g., controller 304, controller 404), and a sensor device (e.g., sensor device 360, sensor device 460).

The processing system 495 may operate substantially continuously (as when the samples 447 and/or the fluid components 427 substantially continuously flow into the processing system 495) or at intervals (as when the samples 447 and/or the fluid components 427 are introduced into the processing system 495 intermittently). The processing system 495 may be or include a single apparatus (with or without multiple portions) or multiple apparatus (or portions thereof) that operate in series and/or in parallel with each other. As an example, the processing system 495 may include a temperature conditioning portion, a mixing portion, a drying portion, and a separating portion that operate in series with each other. As another example, the processing system 495 may include multiple mixers that operate in parallel with each other, where each mixer may mix one or more fluid components 427 and/or one or more of the samples 447 into a different fluid 437 simultaneously.

The processing system 495 may control various aspects (e.g., temperature, pressure, flow rate) of the samples 447, the fluid components 427, and/or the fluid 437. In some cases, the processing system 495 is designed to subject the samples 447, the fluid components 427, and/or the fluid 437 to conditions (e.g., pressure, temperature, flow rate) that simulate the conditions at the subsurface (e.g., corresponding downhole conditions of the fractures 101 and rock matrix 162 in the subterranean formation 110 adjacent to the wellbore 120). The processing system 495 may be controlled by a user 451 (e.g., a human being), by a controller 304 of the system 400, by its own controller (e.g., similar to a controller 304), and/or by a controller 404 of the analytic system 450.

In some cases, some or all of the processing system 495 may be operated, paused, and/or stopped so that the samples 447, the fluid components 427, and/or the fluid 437 may be evaluated by the testing apparatus 470. Testing by the testing apparatus 470 may be controlled by a user 451 (e.g., a human being) and/or a controller 404 of the analytic system 450. Testing by the testing apparatus 470 may be based on historical data and/or field data (e.g., measurements from sensor devices 460). Testing by the testing apparatus 470 may generate test scenarios or expected results. Testing by the testing apparatus 470 may operate using one or more algorithms 533, one or more protocols 532, and/or stored data 534 (all discussed below).

Whether inside the processing system 495 or in a collection area 489 (e.g., a header, a manifold), some or all of the samples 447 and/or some or all of the fluid components 427 may be introduced to each other. Conditions (e.g., temperature, pressure) in some or all of the processing system 495 may vary and may be customized or otherwise controlled (e.g., to represent field operating conditions).

To control the composition of a fluid 437 at a given point in time, the amount of one or more of the samples 447 and/or the amount of the one or more fluid components 427 that are released or withdrawn from the one or more wells 420 and/or the one or more fluid component sources 428, respectively, may be regulated in real time. This regulation may be performed automatically by a controller (e.g., a controller 404 of the analytic system 450, a controller 304) and/or manually by a user 451 (which may include an associated user system 455). This regulation may be performed using equipment such as the processing system 495 (including portions thereof), pumps, compressors, field equipment (e.g., field equipment 109), the conveyance system 488, valves 485, regulators, sensor devices 460, etc. The samples 447 of a well 420 and a fluid component 427 of a fluid component source 428 may have any of a number of different compositions that are naturally occurring, created (e.g., mixed), and/or man-made.

The analytic system 450 of the system 400 may be configured to perform chemistry analysis on the rock 448 in one or more of the samples 447 from one or more of the wells 420. In addition, the analytic system 450 of the system 400 may be configured to perform a chemical and/or other type of analysis of one or more of the subterranean resources 111 from one or more of the wells 420, one or more of the fluid components 427 from one or more of the fluid component sources 428, and/or one or more of the fluids 437 that are configured to be delivered to one or more of the wells 420. As a result, the system 400 (and more specifically the analytic system 450) may be used to evaluate multiple wells 420 using chemistry analysis of the rock 448 in the samples 447, analysis of subterranean resources 111, analysis of the fluid components 427, and/or analysis of the fluids 437. As a result, example embodiments may be used, for example, to optimize the results of a particular field operation (e.g., a fracturing procedure).

As discussed above, the analytic system 450 may include one or more components. For example, in this case, the analytic system 450 includes one or more testing apparatuses 470 and one or more controllers 404. Each testing apparatus 470 of the analytic system 450 may include one or more vessels 461 and one or more sensor devices 460. Each testing apparatus 470 may be configured to test the rock 448 in one or more of the samples 447, one or more of the subterranean resources 111, one or more of the fluid components 427, and/or one or more of the fluids 437. A single testing apparatus 470 may perform multiple tests (e.g., on a single fluid component 427, on samples 447 from a single well 420, on a fluid 437 and on a subterranean resource 111) simultaneously.

When the analytic system 450 has multiple testing apparatuses 470, one testing apparatus 470 may operate in conjunction with, or independently of, one or more of the other testing apparatuses 470. Further, when the analytic system 450 has multiple testing apparatuses 470, one testing apparatus 470 may be configured (e.g., in terms of equipment, in terms of operating capability) the same as, or differently than, one or more of the other testing apparatuses 470. The operation of a testing apparatus 470 may be controlled by a user 451 (including an associated user system 455) and/or a controller 404 of the analytic system 450.

A vessel 461 of a testing apparatus 470 may be configured to retain a solid of a sample 447 for a period of time so that the rock 448 of the sample 447 may be tested and analyzed. A vessel 461 of a testing apparatus 470 may be a natural vessel (e.g., land that forms walls to contain a liquid, a subterranean cavity that holds carbon dioxide or other gas or liquid) and a man-made storage tank or other type of vessel (e.g., a bottle, a column). A vessel 461 of a testing apparatus 470 may be configured to hold a solid, a liquid, and/or a gas. A vessel 461 of a testing apparatus 470 may be configured to accommodate any of a number of parameters (e.g., pressure, temperature, acid or base content) used to receive, test, and/or analyze the rock 448 of a sample 447 and/or an interaction of the rock 448 with one or more fluids (e.g., one or more fluid components 427).

A testing apparatus 470 may include or interact with one or more sensor devices 460 (discussed below) to perform one or more of its functions. Testing performed by a testing apparatus 470 may use or include historical data and/or field data (e.g., measurements from sensor devices 460). Testing may generate test scenarios or expected results. Testing may include the use of process chemistry simulators, fluid electrolyte modeling, chemistry calculations using field/historical data to model the process, etc.

A controller 404 of the analytic system 450 may be configured to evaluate, using results of tests performed by a testing apparatus 470, the samples 447 obtained from one or more wells 420, one or more of the subterranean resources 111 obtained from one or more of the wells 420, one or more of the fluid components 427 obtained from one or more of the fluid component sources 428, and/or one or more of the fluids 437 that are delivered to one or more of the wells 420. For example, a controller 404 of the analytic system 450 may be configured to evaluate, using measurements made by one or more sensor devices 460, a reaction between a rock 448 of a sample 447 with one or more fluid components 427 over a period of time.

A testing apparatus 470 of the analytic system 450 may be configured to process one or more solids in addition to one or more fluids. In such a case, the testing apparatus 470 may be configured to provide analysis of one or more precipitated solids, including but not limited to Fourier transformed infrared spectroscopy (FT-IR), x-ray fluorescence (XRF), x-ray diffraction (XRD), elemental analysis, etc. The testing apparatus 470 may include one or more of any of a number of different equipment, including but not limited to a sifter, a shaker, a screen, a motor, a controller (e.g., controller 404), and a sensor device (e.g., sensor device 460). In some cases, the testing apparatus 470, or portions thereof, may operate using a controller 404. In addition, or in the alternative, one or more users 451 may perform one or more of the various functions required to operate some or all of the testing apparatus 470.

Each testing apparatus 470 of the analytic system 450 may be configured to test the samples 447 of one or more wells 420, one or more fluid components 427, and one or more of the fluids 437. A testing apparatus 470 may be used in conjunction with one or more sensor devices 460 of the analytic system 450. A testing apparatus 470 may be or include a vessel (e.g., a bottle, a column, a test tube) inside of which various materials (e.g., samples 447 from a well 420, a fluid component 427, a fluid 437) are disposed for testing. In some cases, the materials placed in a testing apparatus 470 are first processed in the processing system 495. In any case, the materials are provided to a testing apparatus 470 by the conveyance system 488. A testing apparatus 470 may be used to test samples 447, a fluid component 427, a fluid 437, and/or any other component during a fracturing operation of one or more wells 420, during shut-in of a well 420, during pre-production of a well 420, during production of a well 420, and/or at any other time.

A testing apparatus 470 may include one or more components or pieces of equipment to perform its function. Examples of such components or pieces of equipment may include, but are not limited to, a membrane, a sifter, a shaker, a screen, an immersion separator, a reverse osmosis membrane, a nanofiltration membrane, a pH adjustment apparatus, a softening apparatus, a motor, a controller (e.g., controller 404), a vessel 461, and a sensor device 460. In some cases, a testing apparatus 470, or portions thereof, may operate using a controller 404 of the analytic system 450. In addition, or in the alternative, one or more users 451 (e.g., a human being) may perform some or all of the various functions required to operate some or all of a testing apparatus 470.

Each sensor device 460 of a testing apparatus 470 includes one or more sensors that measure one or more parameters (e.g., pressure, flow rate, temperature, humidity, fluid content, voltage, current, permeability, porosity, characteristics of a rock 448, chemical elements in a fluid, chemical elements in a solid, concentrations, etc.). Examples of a sensor of a sensor device 460 may include, but are not limited to, a temperature sensor, a flow sensor, a pressure sensor, a gas spectrometer, a voltmeter, an ammeter, a permeability meter, a spectrograph, a gas chromatograph a porosimeter, and a camera. A sensor device 460 may be a stand-alone device or integrated with another component (e.g., a vessel 461) of the analytic system 450.

A parameter measured by a sensor device 460 may be associated with a rock 448 of a sample 447. In some cases, in addition, a parameter measured by a sensor device 460 may be associated with one or more other components (e.g., a fluid component 427, a fluid 437) of the system 400. In some cases, a sensor device 460 may additionally or alternatively measure a parameter outside of a vessel 461 of a testing apparatus 470. For example, a sensor device 460 may be configured to measure a parameter (e.g., flow rate, pressure, temperature, mass, porosity, permeability, composition, concentration) of a rock 448 and/or other part of a sample 447, a fluid component 427, and/or a fluid 437 at any location (e.g., between a well 420 and the analytic system 450, between a fluid component source 428 and the analytic system 450, within the processing system 495, etc.) of the system 400 at a particular time.

In some cases, a sensor device 460 of a testing apparatus 470 may be configured to measure a parameter indirectly related to a sample 447, a fluid component 427, and/or a fluid 437. For example, a sensor device 460 may be configured to determine the degree to which a valve 485 within the conveyance system 488 of the system 400 is open or closed. Within a vessel 461, a sensor device 460 may be configured to measure a parameter directly associated with a sample 447, a fluid component 427, and/or a fluid 437. For another example, one or more sensor devices 460 may be used to identify the mass and composition of a solid within a sample 447. As another example, one or more sensor devices 460 may be used to identify the contents of a fluid that is a byproduct of a solid of a sample 447 combined with one or more fluid components 427 within a vessel 461. As yet another example, one or more sensor devices 460 may be used to identify the contents of a fluid component 427.

In some cases, a number of sensor devices 460, each measuring a different parameter, may be used in combination to determine and confirm whether a controller 404 of the analytic system 450 and/or a controller 304 should take a particular action (e.g., operate a valve 485, operate or adjust the operation of a testing apparatus 470, operate or adjust the operation of the processing system 495). When a sensor device 460 includes its own controller 404 (or portions thereof), then the sensor device 460 may be considered a type of computer device, as discussed below with respect to FIG. 6.

The system 400 set forth in FIG. 4 may be used for taking measurements of hydrocarbons released from rock samples after the rock samples interact with a test fluid. For example, certain example embodiments may be directed to a system for improving production performance of a wellbore using a rock sample and rock sample-test fluid interaction testing, where the system may include a fluid source that is configured to provide a test fluid and an analytic system. Such an analytic system may include a testing apparatus and a controller. The testing apparatus of the analytic system may be configured to receive, by the vessel, the rock sample that originates from a portion of a subterranean formation through which the wellbore is drilled; receive, by the vessel, the test fluid from the fluid source; and measure, using the sensor device, a measurement of a hydrocarbon released from the rock sample-test fluid interaction testing in the vessel. The controller of the analytic system may be configured to facilitate generating, using the measurement, a forecast of hydrocarbon production potential for a portion of a subterranean formation from which the rock sample is obtained.

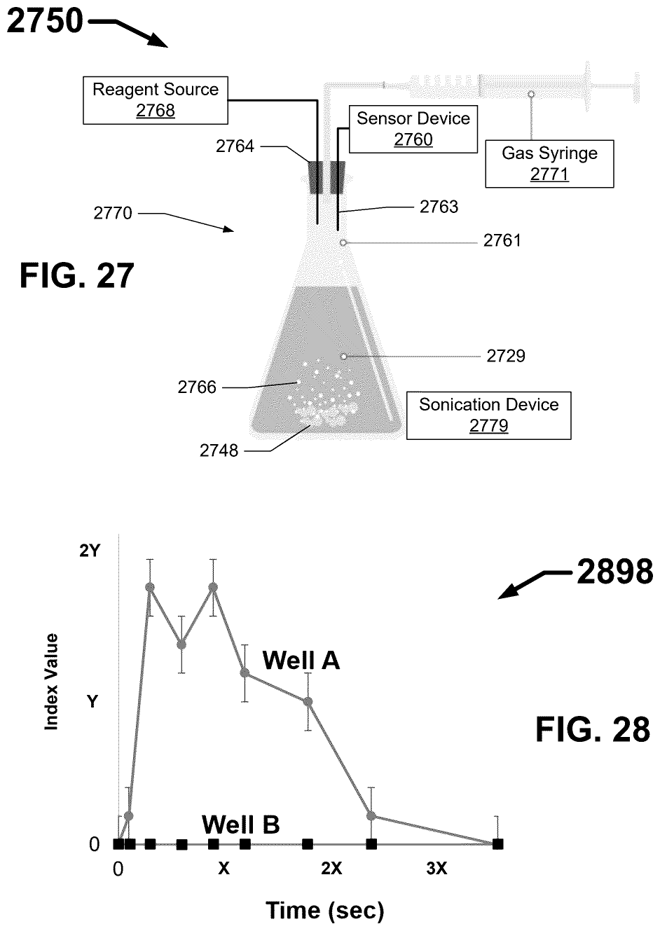

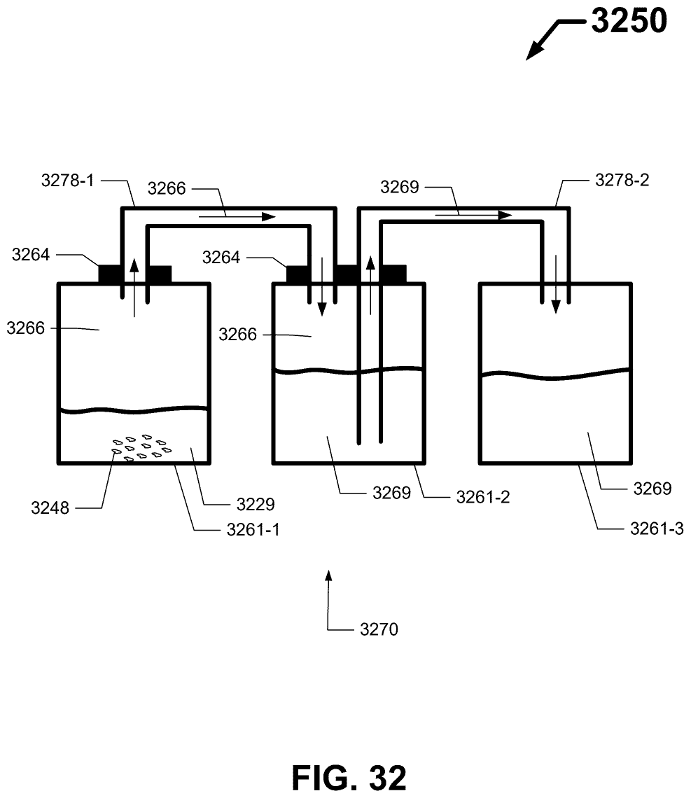

In some cases, the system may also include a processing system configured to process the rock sample before the rock sample is received by the vessel. In such cases, such as discussed below with respect to FIGS. 27 and 32, the processing system may further be configured to process the test fluid before the test fluid is received by the vessel. In some cases, the vessel includes a conical flask. In such cases, the vessel may include a stopper disposed at a top end of the conical flask, where the stopper has an aperture that traverses therethrough, wherein the aperture has disposed therein a tube through which the test fluid is introduced into an interior of the conical flask. In such cases, the stopper may have a second aperture that traverses therethrough, wherein the second aperture has disposed therein a probe for the sensor device. In such cases, the stopper may have a third aperture that traverses therethrough, wherein the third aperture has disposed therein an additional tube through which a chemical reagent is introduced into the interior of the conical flask. In some cases, the vessel may include cotton wool disposed in a neck of the conical flask. In some cases, the vessel of the testing apparatus may be configured to apply a pressure and a temperature to the rock sample and the test fluid. In some cases, the system may also include a sonication device in communication with the vessel, where the sonication device is configured to provide vibrations to the vessel.

As discussed above, the analytic system 450 may include one or more controllers 404. A controller 404 of the analytic system 450 communicates with and in some cases controls one or more of the other components (e.g., a sensor device 460, a testing apparatus 470, another controller 404) of the analytic system 450 and/or one or more other components (e.g., a sensor device 360, a controller 304, the conveyance system 488, one or more valves 485, a fluid component source 428, the processing system 495) of a remainder of the system 400. A controller 404 performs any of a number of functions that include, but are not limited to, obtaining and sending data, evaluating data, following protocols, running algorithms, and sending commands.

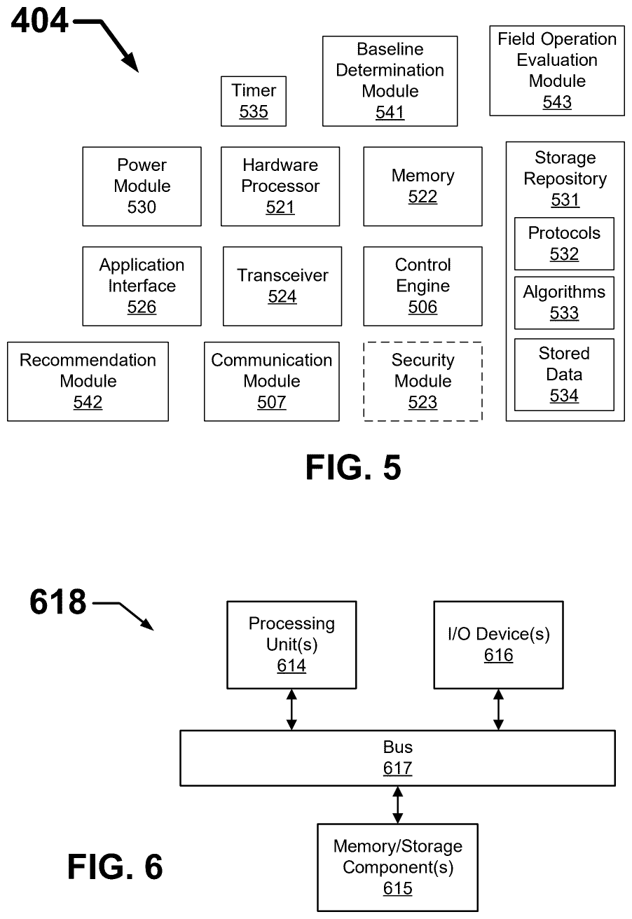

A controller 404 may include one or more of a number of components. For example, as shown in FIG. 5, such components of a controller 404 may include, but are not limited to, a control engine 506, a baseline determination module 541, a recommendation module 542, a field operation evaluation module 543, a communication module 507, a timer 535, a power module 530, a storage repository 531, a hardware processor 521, a memory 522, a transceiver 524, an application interface 526, and, optionally, a security module 523. A controller 404 (or components thereof) may be located at or near the various components of the analytic system 450. In addition, or in the alternative, the controller 404 (or components thereof) may be located remotely from (e.g., in the cloud, at an office building) the various components of the analytic system 450.

When there are multiple controllers 404 (e.g., one controller 404 for a testing apparatus 470, another controller 404 for a fluid component source 428, yet another controller 404 for the processing system 495), each controller 404 may operate independently of each other. Alternatively, two or more of the multiple controllers 404 may work cooperatively with each other. As yet another alternative, one of the controllers 404 may control some or all of one or more other controllers 404 in the system 400 or portion thereof. Each controller 404 may be considered a type of computer device, as discussed below with respect to FIG. 6.

The storage repository 531 may be a persistent storage device (or set of devices) that stores software and data used to assist a controller 404 in communicating with one or more other components of a system, such as the users 451 (including associated user systems 455), each well 420, each fluid component source 428, the processing system 495, the controllers 304, the sensor devices 360, other controllers 404 of the analytic system 450, the network manager 480, the sensor devices 460, etc. of the system 400 of FIG. 4 above. In one or more example embodiments, the storage repository 531 stores one or more protocols 532, one or more algorithms 533, and stored data 534.

The protocols 532 of the storage repository 531 may be any procedures (e.g., a series of method steps) and/or other similar operational processes that the control engine 506 of the controller 404 follows based on certain conditions at a point in time. The protocols 532 may include any of a number of communication protocols that are used to send and/or obtain data between a controller 404 and other components of a system (e.g., the system 400). Such protocols 532 used for communication may be time-synchronized protocols. Examples of such time-synchronized protocols may include, but are not limited to, a highway addressable remote transducer (HART) protocol, a WirelessHART protocol, and an International Society of Automation (ISA) 100 protocol. In this way, one or more of the protocols 532 may provide a layer of security to the data transferred within a system (e.g., the system 400). Other protocols 532 used for communication may be associated with the use of Wi-Fi, Zigbee, visible light communication (VLC), cellular networking, BLE, UWB, and Bluetooth.

The algorithms 533 may be any formulas, mathematical models, forecasts, simulations, and/or other similar tools that the control engine 506 of a controller 404 uses to reach a computational conclusion. For example, one or more algorithms 533 may be used, in conjunction with one or more protocols 532, to assist a controller 404 to determine when to start, adjust, and/or stop the operation of a well 420, a fluid component source 428, a testing apparatus 470, the processing system 495, a sensor device 460, another controller 404 of the analytic system 450 and/or another component of the system 400. As another example, one or more algorithms 533 may be used, in conjunction with one or more protocols 532, to assist a controller 404 to determine when to have a sensor device 460 measure a parameter and subsequently assist the controller 404 in performing a calculation or make a determination using the measurement.

As yet another example, one or more algorithms 533 may be used, in conjunction with one or more protocols 532, to assist a controller 404 to identify an optimal (e.g., most cost effective, most likely to generate a target chemistry of one or more fluid components 427 based on the characteristics and/or composition of the rock 448 in a sample 447) mixture of fluid components 427 to form a fluid 437 that is delivered down a well 420. As still another example, one or more algorithms 533 may be used, in conjunction with one or more protocols 532, to assist a controller 404 to interpret measurements of parameters made by a sensor device 460 after one or more fluid components 427 have been combined with a sample 447 in a vessel 461 of a testing apparatus 470 for a period of time. As yet another example, one or more algorithms 533 may be used, in conjunction with one or more protocols 532, to assist a controller 404 in identifying one or more fluid components 427 that are combined with the rock 448 of a sample 447 in a vessel 461 of a testing apparatus 470.

Stored data 534 may be any data associated with a field (e.g., the subterranean formation 110, the fractures 101 within the volume 190 adjacent to a wellbore 120, the characteristics of proppant 112 used in a field operation, the composition of the water, rock 448, and/or other parts of the samples 447), other fields (e.g., other wellbores and subterranean formations), the other components (e.g., the user systems 455, the testing apparatuses 470, the sensor devices 460, the sensor devices 360, the controllers 304, the fluid components 427, the processing system 495), including associated equipment (e.g., motors, pumps, compressors), of the system 400, measurements made by the sensor devices 460 and the sensor devices 360, threshold values, tables, results of previously run or calculated algorithms 533, updates to protocols 532, user preferences, and/or any other suitable data. Such data may be any type of data, including but not limited to historical data, present data, and future data (e.g., forecasts). The stored data 534 may be associated with some measurement of time derived, for example, from the timer 535.

Examples of a storage repository 531 may include, but are not limited to, a database (or a number of databases), a file system, cloud-based storage, a hard drive, flash memory, some other form of solid-state data storage, or any suitable combination thereof. The storage repository 531 may be located on multiple physical machines, each storing all or a portion of the communication protocols 532, the algorithms 533, and/or the stored data 534 according to some example embodiments. Each storage unit or device may be physically located in the same or in a different geographic location.

The storage repository 531 may be operatively connected to the control engine 506. In one or more example embodiments, the control engine 506 includes functionality to communicate with the users 451 (including associated user systems 455), the testing apparatuses 470, the processing system 495, the sensor devices 460, the sensor devices 360, the controllers 304, the network manager 480, and/or the other components in the system 400. More specifically, the control engine 506 sends information to and/or obtains information from the storage repository 531 in order to communicate with the users 451 (including associated user systems 455), the testing apparatuses 470, the processing system 495, the sensor devices 460, the sensor devices 360, the controllers 304, the network manager 480, and/or the other components of the system 400. As discussed below, the storage repository 531 may also be operatively connected to the communication module 507 in certain example embodiments.

In certain example embodiments, the control engine 506 of a controller 404 controls the operation of one or more components (e.g., the communication module 507, the timer 535, the transceiver 524) of the controller 404. For example, the control engine 506 may activate the communication module 507 when the communication module 507 is in “sleep” mode and when the communication module 507 is needed to send data obtained from another component (e.g., a sensor device 460) in the system 400. In addition, the control engine 506 of a controller 404 may control the operation of one or more other components (e.g., a testing apparatus 470, the processing system 495, a fluid component source 428, operations of a well 420), or portions thereof, of the system 400.

The control engine 506 of a controller 404 may communicate with one or more other components of the system 400 and/or an external system. For example, the control engine 506 may use one or more protocols 532 to facilitate communication with the sensor devices 360 to obtain data (e.g., measurements of various parameters, such as water chemistry, temperature, pressure, and flow rate), whether in real time or on a periodic basis and/or to instruct a sensor device 460 to take a measurement. As another example, the control engine 506 may use one or more algorithms 533 and/or protocols 532 to decide which tests (e.g., determining an ion concentration, determining an ion ratio, determining a mass loss, determining an amount of a stable isotope) to perform on the rock 448 of a sample 447 from a well 420.

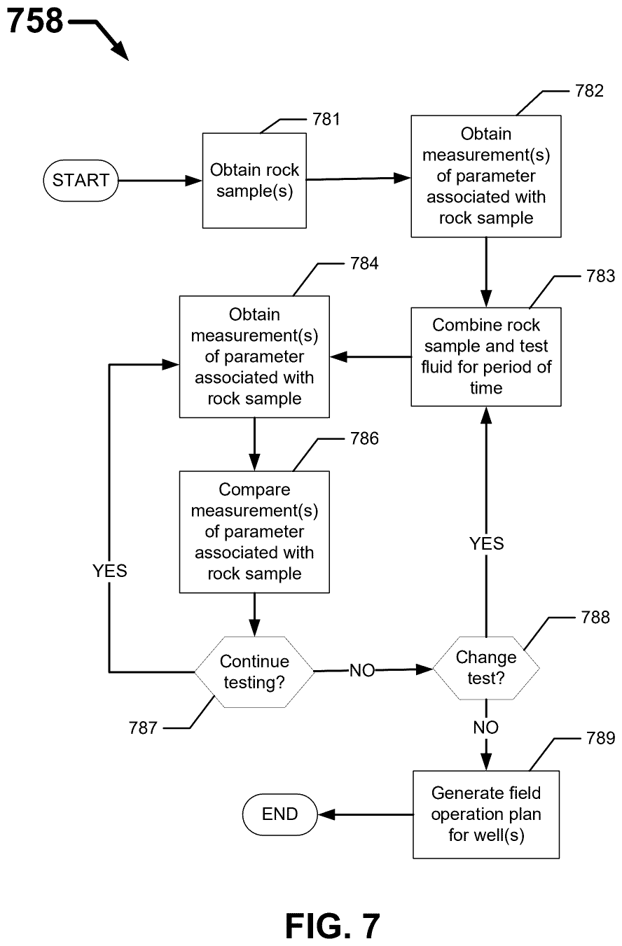

As yet another example, the control engine 506 may use one or more algorithms 533 and/or protocols 532 to generate a new or updated algorithm 533 and/or a new or updated protocol 532 that provides expected results using the baseline for a well 420. As still another example, the control engine 506 may use one or more algorithms 533 and/or protocols 532 to determine, using the results of testing the one or more parameters associated with the rock 448 of samples 447, a volume and composition of a fluid 437 (e.g., a fracturing fluid when the field operation includes fracturing, saltwater when the field operation is injecting the saltwater into a well 420 configured as an injection well). A number of other capabilities of the control engine 506 (as well as the controller 404 as a whole and/or other portions of the controller 404) are discussed below with respect to FIG. 7.

The control engine 506 of the controller 404, through the use of one or more protocols 532 and/or one or more algorithms 533, may implement machine learning as a way to evolve over time with new data and associated changes that may result from the new data. The control engine 506 may use, for example, supervised learning, unsupervised learning, semi-supervised learning, and/or reinforcement learning, as those terms are known in the art of machine learning. In this case, these types of machine learning are effective with sufficient data (e.g., measurements from sensor devices 460) and use of algorithms 533 and/or protocols 532 that automatically build mathematical models using sample data—also known as “training data”.

In this way, for example, the analytic system 450 may measure and interpret the measurements of one or more parameters associated with hydrocarbons emitted from a rock-fluid interaction in order to establish baselines, compare subsequent data to baselines, adjust baselines, perform retroactive analysis, assess a well 420, recommend a landing location for a well 420, recommend placement of a well 420, develop an index (e.g., an indicator, a numeric representation) for the hydrocarbon production potential of a well 420, develop an enhanced oil recovery strategy for a well 420, etc., using data and language elements native to the analytic system 450. Using this flexibility allowed by the learning protocols 532 and/or algorithms 533, the analytic system 450 may scale to disparate vendor solutions and ‘build’ asset development optimization scenarios and recommendations. The learning protocols 532 and/or algorithms 533 may use or include large language models (LLM) to implement unique classification/semantic matching properties that may assist in the development of asset optimization by the analytic system 450.

The learning protocols 532 and/or algorithms 533 that may be used and trained by the control engine 506 may include, but are not limited to, instance-based learning algorithms, artificial neural network algorithms, deep learning algorithms, and ensemble algorithms. Instance-based learning algorithms typically build up a database of example data and compare new data to the database using a similarity measure in order to find a better match and make a prediction. For this reason, instance-based methods are also called winner-take-all methods and memory-based learning. Focus may be put on the representation of the stored instances and similarity measures used between instances. Instance-based algorithms may be computationally expensive for very large datasets since they save all training instances/data points and are sensitive to data noise.

Artificial neural networks may be fairly similar to the human brain. For example, artificial neural networks may be made up of artificial neurons, take in multiple inputs, and produce specific outputs. Artificial neural networks may be an enormous subfield comprised of a large number of neural network architectures and hundreds of algorithms and variations for different types of problems. Artificial neural networks may be biologically inspired computational simulations for certain specific tasks like clustering, classification, or pattern recognition.

Deep learning algorithms may be a modern update to artificial neural networks by building much larger and more complex neural networks. With deep learning, many methods may be applied to very large datasets. Various architectures may be applied for deep learning algorithms. Deep learning may have a high computational cost because much of its development requires advanced processing, storage hardware, and ML platforms/APIs.

Ensemble algorithm methods may be models composed of multiple weaker models that are independently trained and whose predictions are combined in some way to make the overall prediction. Various combination techniques (e.g., averaging, max voting, bagging/bootstrapping (sampling subsets of original complete dataset), boosting) may be applied. Unlike other standard ensemble methods where models are trained in isolation, the boosting technique may employ an iterative approach, training models in succession, with each new model being trained to correct the errors made by the previous ones. Models may be added sequentially until no further improvements may be made.

The control engine 506 may generate and process data associated with control, communication, and/or other signals sent to and obtained from the users 451 (including associated user systems 455), the sensor devices 460, the sensor devices 360, the controllers 304, the other controllers 404 of the analytic system 450, the fluid component sources 428, the conveyance system 488, the network manager 480, and the other components of the system 400. In certain embodiments, the control engine 506 of the controller 404 may communicate with one or more components of a system external to the system 400. For example, the control engine 506 may interact with an inventory management system by ordering replacements for components or pieces of equipment (e.g., a sensor device 460, a valve 485, a motor) within the system 400 that has failed or is failing. As another example, the control engine 506 may interact with a contractor or workforce scheduling system by arranging for the labor needed to replace a component or piece of equipment in the system 400. In this way and in other ways, the controller 404 is capable of performing a number of functions beyond what could reasonably be considered a routine task.

In certain example embodiments, the control engine 506 may include an interface that enables the control engine 506 to communicate with the sensor devices 460, the sensor devices 360, the controllers 304, the other controllers 404 of the analytic system 450, the fluid component sources 428, the conveyance system 488, the user systems 455, the network manager 480, and/or other components of the system 400. For example, if a user system 455 operates under IEC Standard 62386, then the user system 455 may have a serial communication interface that will transfer data to the controller 404. Such an interface may operate in conjunction with, or independently of, the protocols 532 used to communicate between the controller 404 and the users 451 (including corresponding user systems 455), the sensor devices 460, the sensor devices 360, the controllers 304, the other controllers 404 of the analytic system 450, the fluid component sources 428, the conveyance system 488, the network manager 480, and the other components of the system 400.

The control engine 506 (or other components of the controller 404) may also include one or more hardware components and/or software elements to perform its functions. Such components may include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The baseline determination module 541 of the controller 404 may be configured to determine a baseline for the rock 448 within one or more of the samples 447 associated with each of the wells 420. For example, the baseline determination module 541 may use measurements of parameters taken by one or more of the sensor devices 460, where the parameters are associated with the rocks 448 of the samples 447 from a well 420 and/or with a combination of one or more rocks 448 of a sample 447 and one or more fluid components 427 in a vessel 461 of a testing apparatus 470. Using one or more protocols 532 and/or one or more algorithms 533, the baseline determination module 541 may generate a baseline of the parameters associated with the rocks 448 in the samples 447 (e.g., samples 447-1, samples 447-X) for each well 420 (well 420-1, well 420-X). In addition, the baseline determination module 541 may also be configured to modify an existing baseline using measurements of one or more parameters by one or more sensor devices 460, one or more protocols 532, one or more algorithms 533, and/or stored data 534.

Implementation of the functions of the baseline determination module 541 may be performed in one or more of a number of ways. For example, the baseline determination module 541 may determine a difference in at least one parameter between a baseline and results of testing the samples 447 or portions thereof (e.g., rock 448) for at least one of the wells 420 during a field operation, where the difference exceeds a threshold parameter value (e.g., part of the stored data 534) for the at least one parameter.

The recommendation module 542 of the controller 404 may be configured to generate a recommendation regarding one or more of the wells 420. For example, the recommendation module 542 may use one or more protocols 532 and/or one or more algorithms 533 to generate a recommendation as to particular features (e.g., a particular fluid 437 to be used, a flow rate of the fluid 437, a pressure of the fluid 437, a particular well 420 to target, a duration, a start date and time) of a field operation and/or one or more particular wells 420 to direct the field operation. In some cases, the recommendation module 542 may also modify one or more of the features of a field operation that is in progress.

As another example, the recommendation module 542 may use one or more protocols 532 and/or one or more algorithms 533 to recommend an alteration of a chemical composition of a fluid 437 (also sometimes called a field operation fluid) used for a field operation. As yet another example, the recommendation module 542 may use one or more protocols 532 and/or one or more algorithms 533 to recommend an alteration of altering at least one parameter (e.g., a flow rate, a pressure, a temperature) of the field operation fluid 437.

Implementation of the functions of the recommendation module 542 may be performed in one or more of a number of ways. For example, the recommendation module 542 may use one or more algorithms 533 and/or protocols 532 to determine that a difference between the measured results (e.g., as determined by the baseline determination module 541) and the expected results (e.g., based on existing algorithms 533) exceeds a threshold forecast value. In such a case, the recommendation module 542 may use one or more algorithms 533 and/or protocols 532 to generate a revision to the algorithm 533 (e.g., a forecasting model) based on the difference. As another example, the recommendation module 542 may use one or more algorithms 533 and/or protocols 532 to generate a recommendation for a subsequent well 420 added to a pad of existing wells 420. The recommendation module 542 may use stored data 534 that is derived from outputs of the baseline determination module 541.

The field operation evaluation module 543 of the controller 404 may be configured to evaluate a field operation currently being performed or planned to be performed on one or more of the wells 420. For example, the field operation evaluation module 543 may use one or more protocols 532 and/or one or more algorithms 533, as well as measurements of one or more parameters made by one or more sensor devices 460, to compare the results of testing samples 447 from a well 420 during a field operation to expected results generated by one or more algorithms 533 (e.g., a forecasting model). Any differences that exceed a threshold value may be used by the field operation evaluation module 543 as a basis of evaluating the field operation.

Implementation of the functions of the field operation evaluation module 543 may be performed in one or more of a number of ways. For example, the field operation evaluation module 543 may use one or more algorithms 533 and/or protocols 532 to recommend a change to a field operation based on a difference determined between the baseline and the results of testing the samples 447 from one or more wells 420 during the field operation.

The communication module 507 of the controller 404 determines and implements the communication protocol (e.g., from the protocols 532 of the storage repository 531) that is used when the control engine 506 communicates with (e.g., sends signals to, obtains signals from) the user systems 455, the sensor devices 460, the sensor devices 360, the controllers 304, the other controllers 404 of the analytic system 450, the fluid component sources 428, the conveyance system 488, the network manager 480, and the other components of the system 400. In some cases, the communication module 507 accesses the stored data 534 to determine which communication protocol is used to communicate with another component of the system 400. In addition, the communication module 507 may identify and/or interpret the communication protocol of a communication obtained by the controller 404 so that the control engine 506 may interpret the communication. The communication module 507 may also provide one or more of a number of other services with respect to data sent from and obtained by the controller 404. Such services may include, but are not limited to, data packet routing information and procedures to follow in the event of data interruption.

The timer 535 of the controller 404 may track clock time, intervals of time, an amount of time, and/or any other measure of time. The timer 535 may also count the number of occurrences of an event, whether with or without respect to time. Alternatively, the control engine 506 may perform a counting function. The timer 535 is able to track multiple time measurements and/or count multiple occurrences concurrently. The timer 535 may track time periods based on an Instruction obtained from the control engine 506, based on an instruction obtained from a user 451, based on an instruction programmed in the software for the controller 404, based on some other condition (e.g., the occurrence of an event) or from some other component, or from any combination thereof. In certain example embodiments, the timer 535 may provide a time stamp for each packet of data obtained from another component (e.g., a sensor device 460) of the system 400.

The power module 530 of the controller 404 obtains power from a power supply (e.g., AC mains) and manipulates (e.g., transforms, rectifies, inverts) that power to provide the manipulated power to one or more other components (e.g., the timer 535, the control engine 506) of the controller 404, where the manipulated power is of a type (e.g., alternating current, direct current) and level (e.g., 12V, 24V, 120V) that may be used by the other components of the controller 404. In some cases, the power module 530 may also provide power to one or more of the sensor devices 460.

The power module 530 may include one or more of a number of single or multiple discrete components (e.g., transistor, diode, resistor, transformer) and/or a microprocessor. The power module 530 may include a printed circuit board, upon which the microprocessor and/or one or more discrete components are positioned. In addition, or in the alternative, the power module 530 may be a source of power in itself to provide signals to the other components of the controller 404. For example, the power module 530 may be or include an energy storage device (e.g., a battery). As another example, the power module 530 may be or include a localized photovoltaic power system.

The hardware processor 521 of the controller 404 executes software, algorithms (e.g., algorithms 533), and firmware in accordance with one or more example embodiments. Specifically, the hardware processor 521 may execute software on the control engine 506 or any other portion of the controller 404, as well as software used by the users 451 (including associated user systems 455), the network manager 480, and/or other components of the system 400. The hardware processor 521 may be an integrated circuit, a central processing unit, a multi-core processing chip, SoC, a multi-chip module including multiple multi-core processing chips, or other hardware processor in one or more example embodiments. The hardware processor 521 may be known by other names, including but not limited to a computer processor, a microprocessor, and a multi-core processor.

In one or more example embodiments, the hardware processor 521 executes software instructions stored in memory 522. The memory 522 includes one or more cache memories, main memory, and/or any other suitable type of memory. The memory 522 may include volatile and/or non-volatile memory. The memory 522 may be discretely located within the controller 404 relative to the hardware processor 521. In certain configurations, the memory 522 may be integrated with the hardware processor 521.

In certain example embodiments, the controller 404 does not include a hardware processor 521. In such a case, the controller 404 may include, as an example, one or more field programmable gate arrays (FPGA), one or more insulated-gate bipolar transistors (IGBTs), and/or one or more integrated circuits (ICs). Using FPGAs, IGBTs, ICs, and/or other similar devices known in the art allows the controller 404 (or portions thereof) to be programmable and function according to certain logic rules and thresholds without the use of a hardware processor. Alternatively, FPGAs, IGBTs, ICs, and/or similar devices may be used in conjunction with one or more hardware processors 521.

The transceiver 524 of the controller 404 may send and/or obtain control and/or communication signals. Specifically, the transceiver 524 may be used to transfer data between the controller 404 and the users 451 (including associated user systems 455), the sensor devices 460, the sensor devices 360, the controllers 304, the other controllers 404 of the analytic system 450, the fluid component sources 428, the conveyance system 488, the network manager 480, and the other components of the system 400. The transceiver 524 may use wired and/or wireless technology. The transceiver 524 may be configured in such a way that the control and/or communication signals sent and/or obtained by the transceiver 524 may be obtained and/or sent by another transceiver that is part of a user system 455, a sensor device 460, the sensor devices 360, the controllers 304, the other controllers 404 of the analytic system 450, the fluid component sources 428, the conveyance system 488, the network manager 480, and/or another component of the system 400. The transceiver 524 may send and/or obtain any of a number of signal types, including but not limited to radio frequency signals.

When the transceiver 524 uses wireless technology, any type of wireless technology may be used by the transceiver 524 in sending and obtaining signals. Such wireless technology may include, but is not limited to, Wi-Fi, Zigbee, VLC, cellular networking, BLE, UWB, and Bluetooth. The transceiver 524 may use one or more of any number of suitable communication protocols (e.g., ISA100, HART) when sending and/or obtaining signals. The transceiver 524 may send and receive the communication signals using one or more of the communication links 405.

Optionally, in one or more example embodiments, the security module 523 secures interactions between the controller 404, the users 451 (including associated user systems 455), the sensor devices 460, the sensor devices 360, the controllers 304, the other controllers 404 of the analytic system 450, the fluid component sources 428, the conveyance system 488, the network manager 480, and the other components of the system 400. More specifically, the security module 523 authenticates communication from software based on security keys verifying the identity of the source of the communication. For example, user software may be associated with a security key enabling the software of a user system 455 to interact with the controller 404. Further, the security module 523 may restrict receipt of information, requests for information, and/or access to information.

A user 451 may be any person that interacts, directly or indirectly, with a controller 404 and/or any other component of the testing system 400. Examples of a user 451 may include, but are not limited to, a business owner, an engineer, a company representative, a geologist, a consultant, a drilling engineer, a contractor, and a manufacturer's representative. A user 451 may use one or more user systems 455, which may include a display (e.g., a GUI). A user system 455 of a user 451 may interact with (e.g., send data to, obtain data from) the controller 404 via an application interface and using the communication links 405. The user 451 may also interact directly with the controller 404 through a user interface (e.g., keyboard, mouse, touchscreen).

The network manager 480 is a device or component that controls all or a portion (e.g., a communication network, the controller 404) of the system 400. The network manager 480 may be substantially similar to some or all of the controller 404, as described above. For example, the network manager 480 may include a controller that has one or more components and/or similar functionality to some or all of the controller 404. Alternatively, the network manager 480 may include one or more of a number of features in addition to, or altered from, the features of the controller 404. As described herein, control and/or communication with the network manager 480 may include communicating with one or more other components of the same system 400 and/or another system. In such a case, the network manager 480 may facilitate such control and/or communication. The network manager 480 may be called by other names, including but not limited to a master controller, a network controller, and an enterprise manager. The network manager 480 may be considered a type of computer device, as discussed below with respect to FIG. 6.

Interaction between each controller 404, the sensor devices 460, the sensor devices 360, the controllers 304, the other controllers 404 of the analytic system 450, the fluid component sources 428, the conveyance system 488, the users 451 (including any associated user systems 455), the network manager 480, and other components (e.g., the valves 485, the wells 420) of the system 400 may be conducted using communication links 405 and/or power transfer links 487. Each communication link 405 may include wired (e.g., Class 1 electrical cables, Class 2 electrical cables, electrical connectors, Power Line Carrier, RS485) and/or wireless (e.g., Wi-Fi, Zigbee, visible light communication, cellular networking, Bluetooth, Bluetooth Low Energy (BLE), ultrawide band (UWB), WirelessHART, ISA100) technology. A communication link 405 may transmit signals (e.g., communication signals, control signals, data) between each controller 404, the sensor devices 460, the sensor devices 360, the controllers 304, the other controllers 404 of the analytic system 450, the fluid component sources 428, the conveyance system 488, the users 451 (including any associated user systems 455), the network manager 480, and the other components of the system 400.

Each power transfer link 487 may include one or more electrical conductors, which may be individual or part of one or more electrical cables. In some cases, as with inductive power, power may be transferred wirelessly using power transfer links 487. A power transfer link 487 may transmit power between each controller 404, the sensor devices 460, the sensor devices 360, the controllers 304, the other controllers 404 of the analytic system 450, the fluid component sources 428, the conveyance system 488, the users 451 (including any associated user systems 455), the network manager 480, and the other components of the system 400. Each power transfer link 487 may be sized (e.g., 12 gauge, 18 gauge, 4 gauge) in a manner suitable for the amount (e.g., 480V, 24V, 120V) and type (e.g., alternating current, direct current) of power transferred therethrough.

Each of the controllers 304 of the system 400 is a device or component that controls a portion (e.g., a communication network, some of the field equipment 109) of the system 400. A controller 304 may be substantially similar to some or all of the controller 404, as described above. For example, a controller 304 may include a controller that has one or more components and/or similar functionality to some or all of the controller 404. Alternatively, a controller 304 may include one or more of a number of features in addition to, or altered from, the features of the controller 404. As described herein, control and/or communication with a controller 304 may include communicating with one or more other components of the same system 400 and/or another system. In such a case, a controller 304 may facilitate such control and/or communication. Each controller 304 may be considered a type of computer device, as discussed below with respect to FIG. 6.

A user 451 (which may include an associated user system 455), the sensor devices 460, the sensor devices 360, the controllers 304, the other controllers 404 of the analytic system 450, the fluid component sources 428, the conveyance system 488, the network manager 480, and the other components of the system 400 may interact with a controller 404 using the application interface 526. Specifically, the application interface 526 of a controller 404 obtains data (e.g., information, communications, instructions, updates to firmware) from and sends data (e.g., information, communications, instructions) to the user systems 455 of the users 451, the sensor devices 460, the sensor devices 360, the controllers 304, the other controllers 404 of the analytic system 450, the fluid component sources 428, the conveyance system 488, the network manager 480, and/or the other components of the system 400. Examples of an application interface 526 may be or include, but are not limited to, an application programming interface, a web service, a data protocol adapter, some other hardware and/or software, or any suitable combination thereof. Similarly, the user systems 455 of the users 451, the sensor devices 460, the sensor devices 360, the controllers 304, the other controllers 404 of the analytic system 450, the fluid component sources 428, the conveyance system 488, the network manager 480, and/or the other components of the system 400 may include an interface (similar to the application interface 526 of the controller 404) to obtain data from and send data to a controller 404 in certain example embodiments.

In addition, as discussed above with respect to a user system 455 of a user 451, one or more of the sensor devices 460, one or more of the sensor devices 360, one or more of the controllers 304, one or more of the other controllers 404 of the analytic system 450, one or more of the fluid component sources 428, some or all of the conveyance system 488, the network manager 480, and/or one or more of the other components (or portions thereof) of the system 400 may include a user interface. Examples of such a user interface may include, but are not limited to, a graphical user interface, a touchscreen, a keyboard, a monitor, a mouse, some other hardware, or any suitable combination thereof.

The controller 404, the users 451 (including associated user systems 455), the sensor devices 460, the sensor devices 360, the controllers 304, the other controllers 404 of the analytic system 450, the fluid component sources 428, the conveyance system 488, the network manager 480, and the other components of the system 400 may use their own system or share a system in certain example embodiments. Such a system may be, or contain a form of, an Internet-based or an intranet-based computer system that is capable of communicating with various software. A computer system includes any type of computing device and/or communication device, including but not limited to a controller 404. Examples of such a system may include, but are not limited to, a desktop computer with a Local Area Network (LAN), a Wide Area Network (WAN), Internet or intranet access, a laptop computer with LAN, WAN, Internet or intranet access, a smart phone, a server, a server farm, an android device (or equivalent), a tablet, smartphones, and a personal digital assistant (PDA). Such a system may correspond to a computer system as described below with regard to FIG. 6.

Further, as discussed above, such a system may have corresponding software (e.g., user system software, sensor device software, controller software). The software may execute on the same or a separate device (e.g., a server, mainframe, desktop personal computer (PC), laptop, PDA, television, cable box, satellite box, kiosk, telephone, mobile phone, or other computing devices) and may be coupled by the communication network (e.g., Internet, Intranet, Extranet, LAN, WAN, or other network communication methods) and/or communication channels, with wire and/or wireless segments according to some example embodiments. The software of one system may be a part of, or operate separately but in conjunction with, the software of another system within the system 400.

FIG. 6 illustrates one embodiment of a computing device 618 that implements one or more of the various techniques described herein, and which is representative, in whole or in part, of the elements described herein pursuant to certain example embodiments. For example, a controller 404 (including components thereof, such as a control engine 506, a hardware processor 521, a storage repository 531, a power module 530, and a transceiver 524) may be considered a computing device 618 (also called a computer system 618 herein). Computing device 618 is one example of a computing device and is not intended to suggest any limitation as to scope of use or functionality of the computing device and/or its possible architectures. Neither should the computing device 618 be interpreted as having any dependency or requirement relating to any one or combination of components illustrated in the example computing device 618.

The computing device 618 includes one or more processors or processing units 614, one or more memory/storage components 615, one or more input/output (I/O) devices 616, and a bus 617 that allows the various components and devices to communicate with one another. The bus 617 represents one or more of any of several types of bus structures, including a memory bus or memory controller, a peripheral bus, an accelerated graphics port, and a processor or local bus using any of a variety of bus architectures. The bus 617 includes wired and/or wireless buses.

The memory/storage component 615 represents one or more computer storage media. The memory/storage component 615 includes volatile media (such as random access memory (RAM)) and/or nonvolatile media (such as read only memory (ROM), flash memory, optical disks, magnetic disks, and so forth). The memory/storage component 615 includes fixed media (e.g., RAM, ROM, a fixed hard drive, etc.) as well as removable media (e.g., a Flash memory drive, a removable hard drive, an optical disk, and so forth).

One or more I/O devices 616 allow a user 451 to enter commands and information to the computing device 618, and also allow information to be presented to the user 451 and/or other components or devices. Examples of input devices 616 include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques are stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 618 is connected to a network (not shown) (e.g., a LAN, a WAN such as the Internet, cloud, or any other similar type of network) via a network interface connection (not shown) according to some example embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other example embodiments. Generally speaking, the computer system 618 includes at least the minimal processing, input, and/or output means necessary to practice one or more embodiments.

Further, those skilled in the art will appreciate that one or more elements of the aforementioned computer device 618 is located at a remote location and connected to the other elements over a network in certain example embodiments. Further, one or more embodiments is implemented on a distributed system having one or more nodes, where each portion of the implementation (e.g., a fluid component source 428, a testing apparatus 470, the processing system 495) is located on a different node within the distributed system. In one or more embodiments, the node corresponds to a computer system. Alternatively, the node corresponds to a processor with associated physical memory in some example embodiments. The node alternatively corresponds to a processor with shared memory and/or resources in some example embodiments.

The computer device 618 set forth in FIG. 6 may be used for taking measurements of hydrocarbons released from rock samples after the rock samples interact with a test fluid. For example, certain example embodiments may be directed to a computer-implemented method for improving production performance of a wellbore using a rock sample and rock sample-test fluid interaction testing, the computer-implemented method, where the computer-implemented method includes facilitate combining the rock sample and a test fluid for a period of time, where the rock sample originates from a portion of a subterranean formation through which the wellbore is drilled. The computer-implemented method may also include facilitate obtaining a measurement of a hydrocarbon released from the rock sample-test fluid interaction testing after the period of time. The computer-implemented method may further include generating, using the measurement, a forecast of hydrocarbon production potential for a portion of a subterranean formation from which the rock sample is obtained.

FIG. 7 shows a flowchart 758 of a method for asset development optimization using fluid sampling and rock-fluid interaction testing according to certain example embodiments. While the various steps in this flowchart 758 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order. Some or all of the steps of the method of FIG. 7 may be performed off site (e.g., in a laboratory remote from a field operation). In addition, or in the alternative, some or all of the steps of the method of FIG. 7 may be performed on site (e.g., in the field, adjacent to a wellbore 120) where a field operation is being performed or planned.

In addition, a person of ordinary skill in the art will appreciate that additional steps not shown in FIG. 7 may be included in performing this method. Accordingly, the specific arrangement of steps should not be construed as limiting the scope. Further, a particular computing device, such as the computing device 618 discussed above with respect to FIG. 6, may be used to facilitate (e.g., direct, control, provide instructions, provide recommendations, perform, execute) performance of one or more of the steps for the methods shown in FIG. 7 in certain example embodiments. Any of the functions performed below by a controller 404 (an example of which is shown in FIG. 5) may involve the use of one or more protocols 532, one or more algorithms 533, and/or stored data 534 stored in a storage repository 531. In addition, or in the alternative, any of the functions (or portions thereof) in the method may be performed by a user (e.g., user 451).

The method shown in FIG. 7 is merely an example that may be performed by using an example system described herein. In other words, systems for improving production performance using fluid sampling and rock-fluid interaction testing may perform other functions using other methods in addition to and/or aside from those described with respect to FIG. 7. Incorporating the description above with respect to FIGS. 1 through 6, the method shown in the flowchart 758 of FIG. 7 begins at the START step and proceeds to step 781, where rock 448 (also called rock samples 448 herein) from one or more samples 447 from one or more wells 420 are obtained. As used herein, the term “obtaining” may include collecting, receiving, retrieving, accessing, generating, etc. or any other manner of obtaining rock samples 448 from the samples 447 from one or more wells 420. The samples 447 (including the rock 448) may be obtained from some or all of the wells 420 of a pad. The samples 447 (including the rock 448) obtained at this point in time may be prior to or during part (e.g., exploration, production, shut-in period) of a field operation. Each sample 447 may also include some amount of water (e.g., produced water, formation water). Each sample 447 of this step 781 may be called a first sample or an initial sample.

A sample 447 (including the rock 448) may be obtained at the surface (e.g., surface 108, surface 208) using field equipment 109, part of the conveyance system 488, and/or other equipment (e.g., pumps, compressors). For example, a sample 447 may be part of a mud return line, and the cuttings (a form of rock 448) may be included in the drilling mud. In addition, or in the alternative, a sample 447 (including the rock 448) may be obtained directly from the wellbore 420. For example, a sample 447 may be a core sample (another form of rock 448) excised or otherwise extracted from the subterranean formation 110 by a coring tool.

Some or all of the process of obtaining the samples 447 (including the rock 448) from a well 420 may be controlled by a controller 404 (or a collecting component thereof) of the analytic system 450 using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining the samples 447 (including the rock 448) from a well 420 may be controlled by a user 451. The samples 447 (including the rock 448) may be obtained from a well 420 continuously over an extended period of time or on an iterative basis. The rate (e.g., hourly, daily, weekly, randomly) of collecting and testing the rock 448 of samples 447 may vary (e.g., based on field operations, based on field conditions, based on business need, based on whether there is no significant change in the various measured properties of the rock 448) over time. Samples 447 (including the rock 448) from the same well 420 and/or different wells 420 may be obtained from different formation depths.

In some cases, one or more protocols 532 are followed when one or more samples 447 are obtained. For example, when a bottle test is used (e.g., when the vessel 461 of a testing apparatus 470 is in the form of a bottle), samples 447 that include rock 448 in the form of rock cuttings may be obtained for testing. Under the protocol, the rock samples 448 are washed before testing using toluene to remove hydrocarbon contaminants and oil-based mud, followed by repeated washing with toluene 2 or 3 more times before vacuum filtering and air drying the rock samples 448 for at least 12 hours. After that time, as part of step 782 below, the dry rock samples 448 are weighed, and 1-10 g of rock 448 are measured and placed into each vessel 461 (in this case, in the form of a bottle) for testing. In each case, the exact mass of rock 448 in each vessel 461 is weighed and recorded.

Rock samples 448 may be obtained during a drilling operation. Rock samples 448 may be obtained in one or more of a number of ways. For example, one or more rock samples 448 may be obtained through mud filtrate samples, which may include one or more baseline samples and/or rock samples taken from returned mud from various depths in a well 420. As another example, one or more rock samples 448 may be obtained through cuttings following a protocol (e.g., cutting samples collected every 30 feet in a lateral section with a minimal sample size of 4 ounces each). In such cases, the rock samples 448 may have surficial OBM removed and then dried. As yet another example, one or more rock samples 448 may be obtained through core samples, if available. As still another example, one or more rock samples 448 may be obtained through mud gas analysis results, if available.

In step 782, measurements of one or more parameters associated with the rock 448 of the samples 447 are obtained. Measurements of the one or more parameters associated with the rock 448 of the samples 447 may be made, directly or indirectly, using one or more sensor devices 460. The parameters associated with the rock 448 of the samples 447 may be measured at the surface (e.g., surface 108, surface 208). The parameters associated with the rock 448 of the samples 447 that are measured may include, but are not limited to, the composition of the rock 448 of the samples 447, the mass of the rock 448, the amount (concentration) of each part of the composition of the rock, the amount and type of TDSs in the samples 447, the state (e.g., liquid, solid) of each part of the composition, the temperature of the samples 447 (including the rock 448), the permeability of the rock 448, and the porosity of the rock 448.

Some or all of the process of obtaining measurements of the parameters associated with the rock 448 of the samples 447 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining measurements of the parameters associated with the rock 448 of the samples 447 may be controlled by a user 451. The parameters associated with the rock 448 of the samples 447 may be measured continuously over an extended period of time or on a discrete basis.

In some cases, the samples 447 (including the rock 448) may be processed by the processing system 495 before and/or after measurements of the parameters are taken. In the latter case, the parameters associated with the rock 448 of the samples 447 may be remeasured after the samples 447 (including the rock 448) has been processed. The samples 447 (including the rock 448) may be processed multiple times. In addition, or in the alternative, the parameters associated with the rock 448 of the samples 447 may be measured multiple times. The samples 447 may be processed for any of a number of purposes, including but not limited to separating cuttings and/or other forms of rock 448, changing the pH, and adding chemicals (e.g., a fluid component 427). The samples 447 (including the rock 448) may be processed using any of a number of appropriate equipment of the processing system 495, including but not limited to heaters, chillers, mixers, separators, filters, agitators, pumps, and centrifuges.

Some or all of the processing, if any, of the samples 447 (including the rock 448) using the processing system 495 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the processing, if any, of the samples 447 (including the rock 448) using the processing system 495 may be controlled by a user 451. The samples 447 (including the rock 448) may be processed using the processing system 495 continuously over an extended period of time or on a discrete basis.

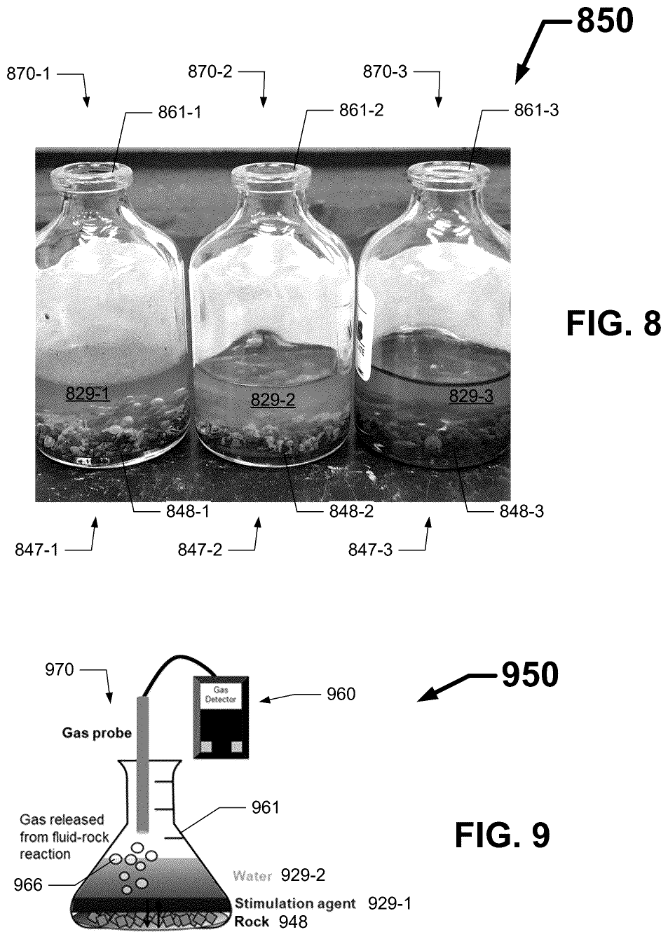

In step 783, the rock 448 and a test fluid 429 (e.g., a single fluid component 427, a combination of multiple fluid components 427) are combined for a period of time. The rock 448 and the test fluid 429 may be combined in a vessel 461 of a testing apparatus 470 of the analytic system 450. The rock 448 and the test fluid 429 may be brought to the vessel 461 using the conveyance system 488, which may be controlled by a controller 404 of the analysis system 450, a controller 304, the network manager 480, and/or a user 451 (including an associated user system 455). The test fluid 429 may be in the form of a liquid and/or a gas. FIGS. 8 through 10, 27, and 32 below show examples of different testing environments and/or set ups the rock 448 and the test fluid 429 may be combined.

One or more of the characteristics (e.g., the amount, the state (e.g., liquid, gas), the chemical composition, the temperature) of the test fluid 429 may be determined and/or implemented by a controller 404 of the analysis system 450, a controller 304, the network manager 480, and/or a user 451 (including an associated user system 455). Similarly, once the rock 448 and the test fluid 429 are combined in the vessel 461, the characteristics (e.g., the pressure, the temperature, humidity, agitation, flow rate) of some or all of the combination (or portions thereof) may be controlled by a controller 404 of the analysis system 450, a controller 304, the network manager 480, and/or a user 451 (including an associated user system 455).

In some cases, the combination (or portions thereof) of the rock 448 and the test fluid 429 may be processed by the processing system 495 during the period of time that the combination is in the vessel 461. In such cases, the combination (or portions thereof) of the rock 448 and the test fluid 429 may be processed continuously over the period of time. Alternatively, the combination (or portions thereof) of the rock 448 and the test fluid 429 may be processed one or more discrete times over the period of time. The combination (or portions thereof) of the rock 448 and the test fluid 429 may be processed for any of a number of purposes, including but not limited to separating cuttings and/or other forms of rock 448, changing the pH, and adding more or different test fluid 429 (e.g., a different fluid component 427).

In some cases, at least some of the processing system 495 may be integrated with or applied to a vessel 461 of a testing apparatus 470 of the analytic system 450. Alternatively, a testing apparatus 470 of the analytic system 450 may include equipment that is functionally similar to the equipment of the processing system 495. Such equipment may include, but is not limited to, a heater, a chiller, a mixer, a separator, a filter, an agitator, a pump, and a centrifuge. Some or all of the processing, if any, of the combination (or portions thereof) of the rock 448 and the test fluid 429 using processing equipment (e.g., from the processing system 495) may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the processing, if any, of the combination (or portions thereof) of the rock 448 and the test fluid 429 may be controlled by a user 451. The combination (or portions thereof) of the rock 448 and the test fluid 429 may be processed continuously over an extended period of time or on a discrete basis.

The period of time that the rock 448 and the test fluid 429 are combined may vary. For example, when the test fluid 429 is a strong reagent, the period of time may be relatively short (e.g., minutes, hours, days). As another example, when the test fluid 429 is a fracturing fluid, the period of time may be relatively longer (e.g., hours, days, weeks, months). The period of time may be driven by the purpose of the test fluid 429. For example, if the purpose of the test fluid 429 is to determine the overall or general viability of the well 420 at the depth from which the rock 448 is obtained, then the period of time may be relatively short and require a small number (e.g., one, two, four) of different test fluids 429. As another example, if the purpose of the test fluid 429 is to generate a specific strategy (e.g., determine the placement of a subsequent well 420, determine a landing zone of the well 420, determine the optimal fracturing fluid for use in a fracturing operation in the well 420), then the period of time may be relatively longer and require a relatively large number (e.g., 4, 6, 11, 100, 398) of different test fluids 429.

In some cases, extending the period of time may be done for a particular strategic purpose. For example, the period of time may be extended to determine whether shutting in the well 420 increases imbibition, and so result in increased hydrocarbon production when the well 420 is put back on production. In such a case, the interaction between the rock 448 and the test fluid 429 may also indicate how long the shut in period should be and/or the composition of the chemicals to be used when the well 420 is put back on production.

In step 784, measurements of one or more parameters associated with the combination (or portions thereof) of the rock 448 and the test fluid 429 are obtained. Measurements of the one or more parameters associated with the combination (or portions thereof) of the rock 448 and the test fluid 429 may be made, directly or indirectly, using one or more sensor devices 460. The parameters associated with the combination (or portions thereof) of the rock 448 and the test fluid 429 may be measured at the surface (e.g., surface 108, surface 208). The parameters associated with the combination (or portions thereof) of the rock 448 and the test fluid 429 that are measured may include, but are not limited to, the composition of the rock 448 within the combination, the mass of the rock 448 within the combination, the amount (concentration) of each part of the composition of the rock 448 within the combination, the amount and type of TDSs in the combination, the state (e.g., liquid, solid) of each part of the combination, the temperature of the combination (including the rock 448), the permeability of the rock 448 within the combination, and the porosity of the rock 448 within the combination.

In some cases, the measurements obtained are associated with hydrocarbons that are released from a reaction of the rock 448 and the test fluid 429. For example, in such cases, measurements may include, but are not limited to, the amount (concentration) of hydrocarbon released, the type of hydrocarbon released, the state (e.g., liquid, solid) of each part of the hydrocarbon released, and the composition of the hydrocarbon released. When the measurements of a parameter are newly obtained, or after a changing event (e.g., a change in the formulation of the test fluid 429, a change in the vessel 461), the measurements may represent or be used to generate a baseline for the rock 448.

Some or all of the process of obtaining measurements of the parameters associated with the combination (or portions thereof) of the rock 448 and the test fluid 429 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining measurements of the parameters associated with the combination (or portions thereof) of the rock 448 and the test fluid 429 may be controlled by a user 451. The parameters associated with the combination (or portions thereof) of the rock 448 and the test fluid 429 may be measured continuously over an extended period of time or on a discrete basis.

In some cases, the combination (including the rock 448) may be processed by the processing system 495 before and/or after measurements of the parameters are taken. In the latter case, the parameters associated with the combination (or portions thereof) of the rock 448 and the test fluid 429 may be remeasured after the combination (including the rock 448) has been processed. Some or all of the combination (including the rock 448) may be processed multiple times. In addition, or in the alternative, the parameters associated with the combination (or portions thereof) of the rock 448 and the test fluid 429 may be measured multiple times. The combination (or portions thereof) of the rock 448 and the test fluid 429 may be processed for any of a number of purposes, including but not limited to separating cuttings and/or other forms of rock 448, changing the pH, and adding more chemicals (e.g., an additional fluid component 427). The combination (or portions thereof) of the rock 448 and the test fluid 429 may be processed using any of a number of appropriate equipment of the processing system 495, including but not limited to heaters, chillers, mixers, separators, filters, agitators, pumps, and centrifuges.

Some or all of the processing, if any, of the combination (or portions thereof) of the rock 448 and the test fluid 429 using process equipment (e.g., processing equipment of the processing system 495, processing equipment integrated with the vessel 461) may be controlled by a controller 404 (or a processing component thereof) of the analytic system 450 using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the processing, if any, of the combination (or portions thereof) of the rock 448 and the test fluid 429 may be controlled by a user 451. The combination (or portions thereof) of the rock 448 and the test fluid 429 may be processed continuously over an extended period of time or on a discrete basis.

In some cases, one or more protocols 532 are followed when multiple rock samples 448 are tested and measured. For example, when a bottle test is used (e.g., when the vessels 461 of a testing apparatus 470 are in the form of bottles), each rock sample 448 may be treated and dried in preparation for testing, as discussed above with respect to steps 781 and 782. A different test fluid 429 in the form of one or more different solutions and/or stimulation chemicals (e.g., synthetic brine solutions, field brine solutions, hydrochloric acid (1-15% by weight), acetic acid (1-15% by weight), nitric acid, mud acid, scale inhibitors, iron control agents, etc.) may be chosen to combine with a rock sample 448 in each vessel 461. FIG. 11 below shows an example of a graph showing mass loss of rock samples 448 for different test fluids 429.

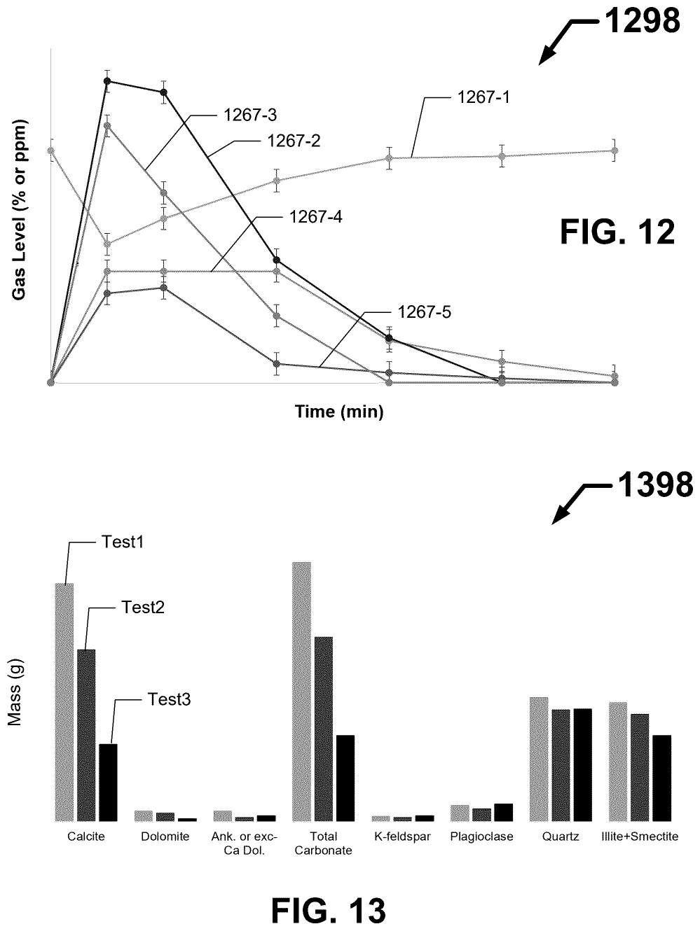

A sensor device 460 in the form of a custom gas detection monitor may be suspended above the test fluid 429 of each vessel 461 to monitor gas (e.g., gas 966 in FIG. 9 below) released by the rock-fluid interactions. The sensor device 460 may be capable of detecting, for example, CO₂, CO, H₂S, SO₂, O₂, and combustible gases (e.g., H₂, hydrocarbons). FIG. 12 below shows an example of a graph plotting various gases released by rock-fluid interactions. In some cases, after some exposure time (e.g., 1 minute, 8 weeks), the rock-fluid reaction is quenched by the addition of another test fluid 429 in the form of a brine or pure deionized water if the initial test fluid 429 was an acid.

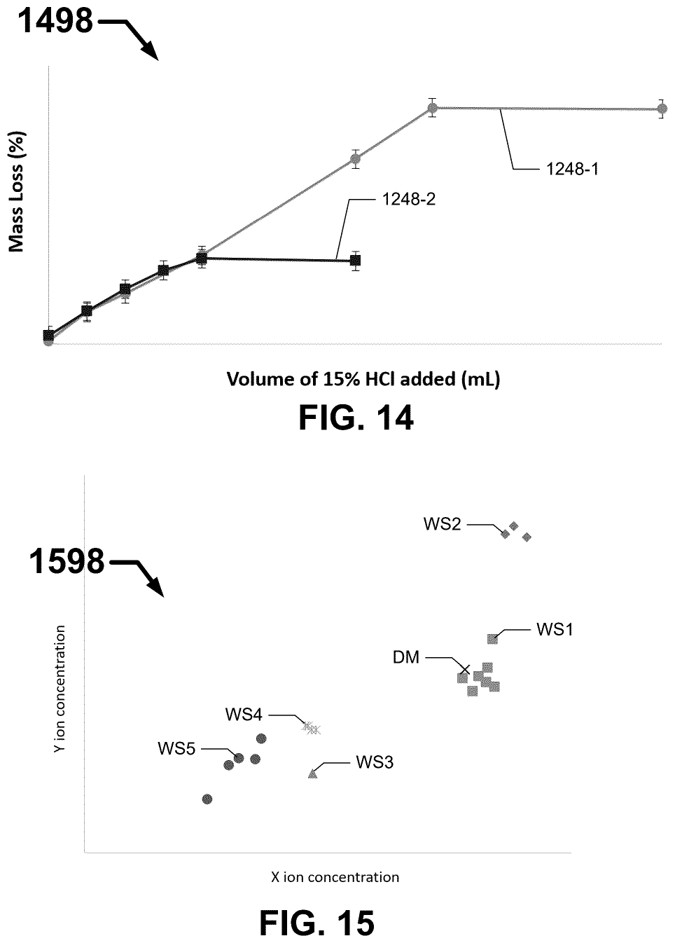

The resulting combination may be collected and syringe filtered through a 0.45 μm filter. A pH value of the remaining fluid may be determined to evaluate consumption of added acid. In some cases, fluid samples may then be acid preserved using 1-2 drops of concentered nitric acid to prevent precipitation of dissolved species. The resulting mixture may then be analyzed by various methods such as ICP-MS and IC to determine dissolved cation and anion levels. Rock samples may be analyzed by methods such as quantitative x-ray diffraction (QXRD) and XRF before and after fluid interaction to determine changes in minerology and elemental composition. An example of this is shown in the graph of FIG. 13 below. Rock mass loss after the rock-fluid interaction may also be determined, for example, by vacuum filtration, air drying overnight, and mass measurement of recovered rock cutting fragments the following morning. An example of this is shown in the graph of FIG. 14 below.

Rock-water or rock-acid reactions may be monitored over time (with or without dynamic gas release monitoring). The reaction may be quenched (i.e., stopped) at different intervals for chemical analysis of spent reaction fluid one or more analytical methods. Chemical analysis post-reaction of rock mineralogical changes and dissolved species in the fluid allows for determination of field rock properties and potential interactions with fracturing and/or stimulation agents. These results using example embodiments may directly aid characterization of unique and/or individual field rock and/or formation properties for production chemistry risk assessments. These results using example embodiments may also allow for tailored chemical usage recommendations to protect and improve subsurface integrity and ROI from unconventional and other assets as appropriate.

In some cases, a mud filtrate analysis and data interpretation may be performed on one or more rock samples 448. In such cases, different testing methods (e.g., inductively coupled plasma (ICP), IC testing, stable isotope testing). Also in such cases, data interpretation and comparison may be made with existing water chemistry data. In addition, or in the alternative, rock-fluid interaction tests may be performed on one or more rock samples 448. In such cases, the tests may reveal the composition (e.g., H₂, CO₂, H₂S, CO, SO₂, hydrocarbons) and the amount of gas released from the rock sample 448. In addition, or in the alternative, in such cases, the tests may reveal cations, anions, elements, hydrocarbons, and the like released into the fluid phase (e.g., using ICP testing, IC testing, stable isotope testing). In some cases, the test fluid 429 may be an acidic solution (e.g., containing HCl, CH₃COOH, etc.) or a non-acidic solution (e.g., fracturing water, fracturing fluid).

In step 786, the measurements of the parameters associated with the rock sample 448 over time are compared. A comparison of the measurements of the parameters associated with the rock sample 448 may be made by a controller 404 (or a comparing component thereof (e.g., the baseline determination module 541)) of the analytic system 450 using one or more protocols 532, one or more algorithms 533 (e.g., models), and stored data 534. Alternatively, a comparison of the measurements of the parameters associated with the rock sample 448 may be made by a controller 304 of the system 400, the network manager 480, a user 451 (including an associated user system 455), or some other component of the system 400. The comparisons may result in or contribute to a trend, the establishment of a baseline, the creation or modification of an algorithm 533, and/or some other predictive indication associated with the rock 448. In certain example embodiments, this step 786 may be used to establish a calibration or reference database to help forecast and/or improve reservoir and well performance in TRU plays.

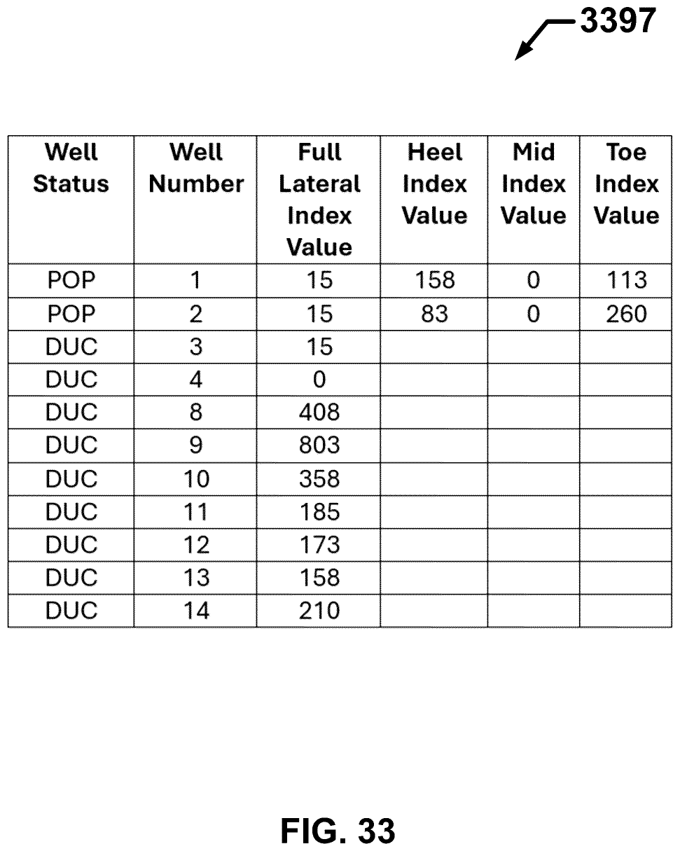

When the measurements are compared to a baseline, the result may be used to assess (e.g., determine the hydrocarbon production potential of) the well 420 from which the rock 448 is obtained and/or adjacent wells 420. The comparison of the measurements to a baseline may be a high-level assessment (e.g., develop further, stop development, land a lateral section at a specific depth) of a well 420. Alternatively, the comparison of the measurements to a baseline may be an optimization assessment (e.g., fracture the well 420 at a depth or range of depths using a particular fracturing fluid for a particular duration of time). The comparison of the measurements to a baseline may be an assessment of hydrocarbon production potential for the well 420 and/or lead to the development of an enhanced oil recovery (EOR) strategy. The comparison of the measurements to a baseline may be quantified as some value (e.g., an index (see, e.g., FIG. 33)) or range of values that can be compared to a threshold value.

In some cases, rather than using a baseline as a basis of comparison, the measurements are compared with other measurements (e.g., of the same well 420, of another well 420). In such cases, the comparison may lead to an overall strategy or a specific strategy (e.g., a put on production (POP) sequence, well-well interaction, fracturing and/or refracturing details, a chemical injection plan and related details, chemical treatment versus fracturing operation, well performance lookback for reservoir performance) that includes multiple wells 420.

In some cases, multiple different tests (e.g., different test fluids 429 applied to the rock 448 and/or the rock sample-test fluid interaction being conducted in different vessels 461 (e.g., under different configurations and/or conditions)) are conducted in parallel according to certain example embodiments. In such cases, the results may additionally or alternatively be compared with each other (as opposed to only being compared against a baseline). This approach may be used, for example, when determining an optimal enhancement operation for a well 420 that has been determined to be viable (e.g., has an index that exceeds a threshold value).

In step 787, a determination is made as to whether testing of the rock 448 should continue. Such a determination may be based on whether any of a number of factors, including but not limited to whether another field operation will be performed on the same and/or a different well 420, whether the status of a well 420 changes (e.g., from shut-in to not shut-in, from current status to further development (e.g., another fracturing operation, chemical treatment)), the amount of time that has passed since the field operation ended, after a new landing spot of a well has been developed, and the amount of change in the current test results relative to previous results and/or the baseline for the rock 448 of a well 420. The determination may be made by a controller 404 (or the recommendation module 542 thereof) of the analytic system 450 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the determination may be made by a user 451. The determination may be made at the surface (e.g., surface 108, surface 208). If the rock 448 continues to be tested, then the process reverts to step 784. If the rock 448 does not continue to be tested, then the process proceeds to step 788.

In step 788, a determination is made as to whether the testing of the rock 448 should be changed. The testing of the rock 448 may be changed in any of a number of ways. Examples of the ways in which testing of the rock 448 may be changed may include, but are not limited to, adding a new test fluid 429, changing (e.g., reducing, increasing, removing) the current test fluid 429, changing a condition (e.g., temperature, pressure, agitation) within the vessel 461, increasing the period of time for the test, and adding and/or removing a parameter associated with the rock 448 to be measured. For example, if testing is being conducted to identify the optimal fracturing fluid to be used in a secondary fracturing operation, each test fluid 429 may be a different fracturing fluid, and the test environment may be controlled to mirror one or more aspects (e.g., temperature, pressure, flow rate) of the downhole environment. For another example, if testing is being conducted to identify a potential chemical treatment (e.g., an acid treatment, surfactant treatment, polymer treatment) of the part of the subterranean formation from which the rock 448 is obtained, each test fluid 429 may be a different chemical treatment fluid, and the test environment may be controlled to mirror one or more aspects (e.g., temperature, pressure, flow rate) of the downhole environment.

The determination may be made by a controller 404 (or the recommendation module 542 thereof) of the analytic system 450 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the determination may be made by a user 451. The determination may be made at the surface (e.g., surface 108, surface 208). If the testing of the rock 448 is not changed, then the process reverts to step 783. If the testing of the rock 448 is changed, then the process proceeds to step 789.

In step 789, a field operation plan for one or more wells 420 is generated. In certain example embodiments, the field operation plan uses a recommended operation fluid that is based on comparing the measurements of the parameters associated with the rock sample 448. The field operation plan may be generated by a controller 404 (or the field operation evaluation module 543 thereof) of the analytic system 450 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the field operation plan may be generated by a user 451. The field operation plan may be generated at the surface (e.g., surface 108, surface 208).

Example embodiments may be applied to different types of subterranean formations (e.g., subterranean formation 110, subterranean formation 210). Factors to consider in such cases may include, but are not limited to, the type of formation, size and/or type of cuttings, types of rock samples (e.g., cuttings, core samples), other rock-related data (e.g., well logs, geology data, production data, engineering, data, operation fluids 437 (including chemical additives) placed into a subterranean formation during a fracturing and/or other type of field operation) available, and the impact of rock permeability and/or porosity. Example embodiments may also utilize systematic rock-fluid interaction studies. Examples of such rock-fluid interaction studies may include, but are not limited to, any water and/or chemical additives that may be injected into a subterranean formation, the impact on oil and water fingerprinting analysis, the impact on fluid chemistry-related risks (e.g., scale, corrosion, sludge), and a comprehensive characterization of hydrocarbons (e.g., gas, liquid) released.

In some cases, generating a field operation plan also includes implementing or facilitating the implementation of a field operation. For example, if a field operation plan includes a recommended operation fluid for a field operation to be performed on a well 420, then a controller 404 (or the field operation evaluation module 543 thereof) of the analytic system 450 may control one or more of the fluid component sources 428 and parts of the conveyance system 488 to generate a fluid 437 that matches the characteristics (e.g., chemical composition, gas/liquid state, volume) of the of the recommended operation fluid from the field operation plan. In some cases, in addition, a controller 404 (or the field operation evaluation module 543 thereof) of the analytic system 450 may control the delivery (e.g., flow rate, temperature, pressure) of the fluid 437 into the one or more wells 420 targeted in the field operation plan.

Example embodiments may be used to update the economic assessment of one or more wells 420. In such cases, example embodiments may generate one or more indicators, including but not limited to a forecast on water and/or oil production, a recommended intervention of a well 420, and a recommendation as to whether a fracturing operation performed on a well 420 is worthwhile. In addition, or in the alternative, example embodiments may be used to modify a fracturing design for a well 420. In such case, the modified fracturing design may optimize, for example, the fracturing fluid, recommend a chemical to control iron levels, skip one or more sections along a lateral, modify a plug distance and/or location, and optimize a perforation location and/or frequency. When step 789 is complete, the process proceeds to the END step.

The method described in FIG. 7 may capture a number of different variations of a method for improving production performance using fluid sampling and rock-fluid interaction testing. For example, in one example embodiment, the method may include: (1) collecting mud filtrate samples for the drilling mud used and for the returned drilling mud (corresponding to step 781); (2) conducting a press test to investigate production section waterflows and to predict produced water sources/water cut at the production stage (corresponding to step 782); (3) generating and implementing criteria to select appropriate rock (core or cutting) samples 448 from reservoirs/wells 420 (including both producing wells and drilled but not completed wells) (corresponding to part of step 781); (4) conducting rock-fluid interaction experiments, where the fluid includes acid and aqueous solutions (corresponding to step 783): (5) monitoring/sampling the tests, which include but not limited to rock mass loss, dissolved ions, generated gas composition, and stable isotope analysis of both liquid and gas samples (corresponding to step 784); (6) integrating the analysis results to establish a rock-fluid interaction profile/database (corresponding to step 786); (7) correlating the test results and fluid chemistry assessments to formation properties (e.g., porosity/permeability/mineralogy data), production data (e.g., production rate, GOR, cumulative oil/water/gas production, etc.), well logging results, and other data sources (e.g., mud gas data) (corresponding to step 786); (8) comparing/correlating results from cutting samples from the well 420 to results from core samples from the well 420 (corresponding to step 786); (9) developing a field operation plan by identifying key factors and establishing a workflow with key indexes and “calibration” envelope/algorithm to assess/forecast/improve TRU reservoir and individual well performance (corresponding to step 789); and (10) utilizing test results from rock and fluid samples collected during drilling stages for both ongoing and future developments to modify the field operation plan as needed (corresponding to step 787).

In another example embodiment, the method may include: (1) collecting rock samples 448 (corresponding to step 781); (2) measuring parameters associated with rock-fluid interaction using a variety of experimental setups, including but not limited to bottle test, autoclave, coreflood, aging cell, and Amott cell (corresponding to steps 783 and 784); (3) adding fluid and chemical additives that may be placed into the formation (corresponding to steps 783, 787, and 788); (4) measure parameters using a full suite of fluid chemistry monitoring for the rock-fluid reaction (corresponding to step 784); (5) monitoring and analyzing compositions gas and hydrocarbons released during the rock-fluid interaction (e.g., H₂, CO₂, H₂S, CO, SO₂, hydrocarbons) (corresponding to steps 784 and 786); (6) performing stable isotope analysis of both liquid and gas samples as appropriate (corresponding to steps 784 and 786); and (7) developing an integrated test system incorporating in-situ rock-fluid interaction/gas generation experiments with gas composition or stable isotope analysis (including but not limited to gas detector, GC, GC-MS) (corresponding to steps 783 through 788).

The method set forth in the flowchart 758 of FIG. 7 may apply to the measurements of hydrocarbons released from rock samples after the rock samples interact with a test fluid. For example, certain example embodiments may be directed to a method for improving production performance of a wellbore using a rock sample and rock sample-test fluid interaction testing. In such a case, the method may include combining the rock sample and a test fluid for a period of time, where the rock sample originates from a portion of a subterranean formation through which the wellbore is drilled. Such a method may also include obtaining a measurement of a hydrocarbon released from the rock sample-test fluid interaction testing after the period of time. Such a method may further include generating, using the measurement, a forecast of hydrocarbon production potential for a portion of a subterranean formation from which the rock sample is obtained.

In such cases, the hydrocarbon may include a liquid and/or a gas. In such cases, the test fluid may include an acid, a brine, chemical additives, formation water, and/or some other chemical. When the test fluid includes an acid, the acid may include at least one of a group consisting of HCl, HNO₃, acetic acid, CO₂, and saturated acidic water. The test fluid may be modified based on how the well is completed and treated under field conditions. For hydraulic fracturing technology, aqueous phase fluid may be utilized. In addition to the reagents used to interact with rock samples, some or all of the chemical additives potentially used during completion, refracturing, and/or well stimulation may be used as at least part of a test fluid.

A rock sample may be among drilling mud circulated to a surface from the wellbore. A rock sample may be or include a core sample extracted from the wellbore. A rock sample may be collected from a substantially vertically oriented portion of the wellbore. In such cases, the method for improving production performance of a wellbore using a rock sample and rock sample-test fluid interaction testing may also include determining, using the measurement, a location to land a subsequent horizontal wellbore.

A rock sample may be collected from a substantially horizontally oriented portion of the wellbore. In such cases, the method for improving production performance of a wellbore using a rock sample and rock sample-test fluid interaction testing may also include determining, using the forecast of the hydrocarbon production potential, a production forecast for the wellbore. In addition, or in the alternative, in such cases, the method for improving production performance of a wellbore using a rock sample and rock sample-test fluid interaction testing may also include determining, using the forecast of the hydrocarbon production potential, a completion optimization plan for the wellbore. In addition, or in the alternative, in such cases, the method for improving production performance of a wellbore using a rock sample and rock sample-test fluid interaction testing may also include assessing and prioritizing existing wells for further stimulation, refracturing, and/or other treatments.

While strong reagents may be used to accelerate reaction for landing optimization purposes, for completion optimization purposes, fracturing fluids with chemical additives may be used. Current data and conditions may be used to determine the amount of time for reactions, the chemical dosages for test fluids, the chemical type for test fluids, etc. that may result in more or optimal hydrocarbon gas release from the rock samples during interaction with the test fluids.

A measurement made during the method for improving production performance of a wellbore using a rock sample and rock sample-test fluid interaction testing may be obtained using, for example, gas chromatography, an oil in water analyzer, by measuring a mass of the hydrocarbon, using water displacement, and using a catalytic bead sensor. In such cases, the measurement of the hydrocarbon may include a chemical content of a gas and a volume of the gas.

FIG. 8 shows part of an analytic system 850 in accordance with certain example embodiments. Incorporating the description above with respect to FIGS. 1 through 7, the part of the analytic system 850 shown in FIG. 8 includes parts of three testing apparatuses 870. Testing apparatus 870-1 includes a vessel 861-1 in the form of a bottle. Inside and at the bottom of the vessel 861-1 is a sample 847-1 that includes rock 848-1 and a test fluid 829-1 in the form of a liquid. Testing apparatus 870-2 includes a vessel 861-2 in the form of a bottle. Inside and at the bottom of the vessel 861-2 is a sample 847-2 that includes rock 848-2 and a test fluid 829-2 in the form of a liquid. Testing apparatus 870-3 includes a vessel 861-3 in the form of a bottle. Inside and at the bottom of the vessel 861-3 is a sample 847-3 that includes rock 848-3 and a test fluid 829-3 in the form of a liquid.

Using a vessel 861 in the form of a bottle allows for testing that is easily scalable in size and number for testing large libraries of rock 848 (e.g., drill cutting sample). For example, a test on rock 848 may involve dozens or hundreds of vessels 861 in the form of bottles. Rock 848 in the form of drill cutting samples may be initially evaluated for size/morphology and mineralogical content. Subsequently, the rock 848 may be exposed to different test fluids 829 in the form of aqueous solutions and stimulation fluids (e.g., hydrochloric acid, acetic acid, nitric acid, mud acid etc.) at customizable rock mass-to-fluid volume ratios.

FIG. 9 shows part of another analytic system 950 in accordance with certain example embodiments. Incorporating the description above with respect to FIGS. 1 through 8, the part of the analytic system 950 shown in FIG. 9 includes parts of a single testing apparatus 970. Specifically, the testing apparatus 970 of FIG. 9 includes a vessel 961 in the form of a bottle and a sensor device 960 in the form of a gas detector. Disposed within the vessel 961 is a rock sample 948. Combined with the rock 948 in the vessel 961 is a test fluid 929-1 in the form of a stimulation agent (e.g., an acid). Another test fluid 929-2 in the form of water is also inside the vessel 961, positioned above the test fluid 929-1. In alternative cases, the test fluid 929-2 is omitted from the test.

The interaction between the rock 948 and the test fluid 929-1 yields a gas 966, which rises through the test fluid 929-2 and is detected by the sensor device 960. For example, there may be a rapid chemical reaction and evolution of mixed gases by combining the rock 948 and the test fluid 929-1 in the vessel 961, which may be detected and broken down into relative percent components (e.g., O₂, H₂, CO₂, H₂S, CO, SO₂, and combustible gases such as hydrocarbons) using the sensor device 960. The test fluid 929-1 that is spent mixed with the test fluid 929-2 in the form of water may then be analyzed (e.g., using another sensor device 960) for dissolved chemical species (e.g., cations, anions) by various water chemistry analyses.

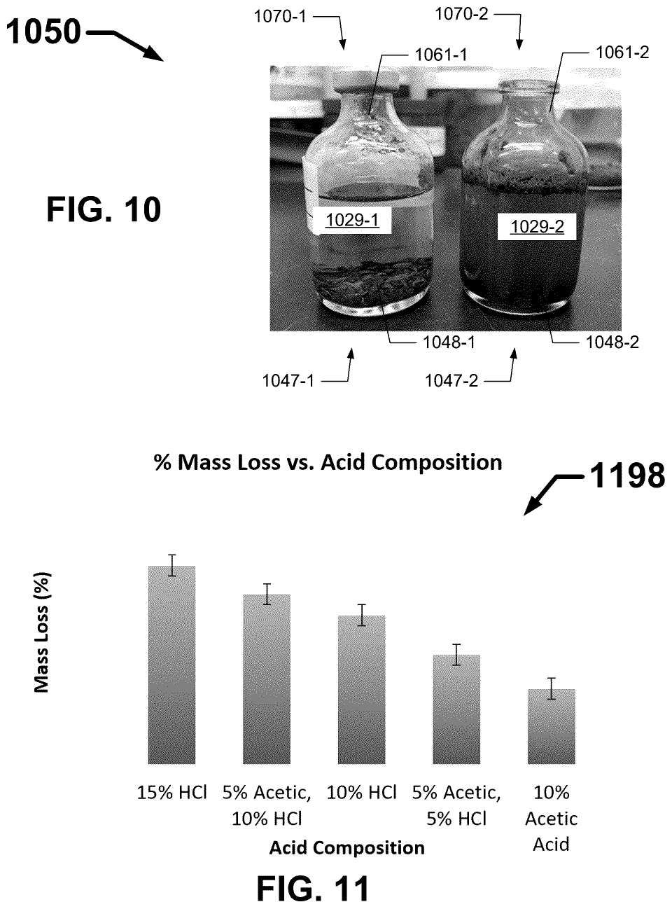

FIG. 10 shows part of another analytic system 1050 in accordance with certain example embodiments. Incorporating the description above with respect to FIGS. 1 through 9, the part of the analytic system 1050 shown in FIG. 10 includes parts of three testing apparatuses 1070. Testing apparatus 1070-1 includes a vessel 1061-1 in the form of a bottle. Inside and at the bottom of the vessel 1061-1 is a sample 1047-1 that includes rock 1048-1 and a test fluid 1029-1 in the form of a brine. Testing apparatus 1070-2 includes a vessel 1061-2 in the form of a bottle. Inside and at the bottom of the vessel 1061-2 is a sample 1047-2 that includes rock 1048-2 and a test fluid 1029-2 in the form of hydrochloric acid. The rock 1048-1 and the rock 1048-2 may be taken from the same sample 1047. There is significantly more rock-fluid interaction in the vessel 1061-2 compared to the rock-fluid interaction in the vessel 1061-1. As a result, there is rock reaction/dissolution into the fluid phase in the vessel 1061-2, allowing for analytical testing of release agents and dissolved ions.

FIG. 11 shows a graph 1198 of mass loss of rock samples 448 according to certain example embodiments. Incorporating the description above with respect to FIGS. 1 through 10, the graph 1198 shows mass loss of a rock sample 448 as a percentage along the vertical axis and acid composition along the horizontal axis. This graph 1198 shows that customizing the test fluid 429 may determine how much mass loss a rock sample 448 experiences. When the test fluid 429 includes 15% HCl, the mass loss of the rock sample 448 is higher than the mass loss for when the test fluid 429 includes 5% acetic acid and 10% HCl, which is higher than when the test fluid 429 includes 10% HCl, which is higher than when the test fluid 429 includes 5% acetic acid and 5% HCl, which is higher than when the test fluid 429 includes 5% acetic acid.

FIG. 12 shows a graph 1298 of a gas analysis resulting from a rock-fluid interaction according to certain example embodiments. Incorporating the description above with respect to FIGS. 1 through 11, the graph 1298 of FIG. 12 plots the gas level along the vertical axis versus time in minutes along the horizontal axis for 5 different gases (e.g., gas 966) that result from the interaction of a rock sample 448 and a test fluid 429 in a vessel 461. The levels of gas may be measured by a sensor device 460 of a testing apparatus 470 of the analytic system 450. Plot 1267-1 represents the level of O₂ as a percentage over time.

To standardize and/or further develop the implementation of example embodiments, one or more of a number of processes may be followed. For example, some or all of the testing may be automated to result in automatic and/or more frequent logging of the gas monitoring results. As another example, data interpretation may be optimized by utilizing peak height, peak width, integrated areas, and/or other data metrics in the comparison and other analysis. As yet another example, the amount of rock, the rock-acid ratio in the gas release profile, and/or other factors may be investigated. As still another example, by analyzing the impact of the size of cutting samples and/or crushed rock samples on the gas release profile, samples with similar size ranges may be selected for further testing and analysis.

FIG. 13 shows a graph 1398 of minerology changes in rock 448 before and after interaction with a test fluid 429 according to certain example embodiments. Incorporating the description above with respect to FIGS. 1 through 12, the graph 1398 of FIG. 13 shows the mass (in grams) of calcite, dolomite, Ank.or exc-Ca Dol., total carbonate, K-feldspar, plagioclase, quartz, and illite+smectite in a rock sample 448 at 3 stages. The first test (Test1) is shown in the graph 1398 as the left-most vertical bar for each item along the horizontal axis and occurs before the interaction between the rock sample 448 and a test fluid 429 in the form of HCl. The second test (Test2) is shown in the graph 1398 as the middle vertical bar for each item along the horizontal axis and occurs when the test fluid 429 in the form of HCl is at 4 mL. The third test (Test3) is shown in the graph 1398 as the right-most vertical bar for each item along the horizontal axis and occurs when the test fluid 429 in the form of HCl is at 8 mL. These measurements are made using a sensor device 460 using quantitative x-ray diffraction (QXRD) technology. The results of the graph 1398 show a change in the minerology of the rock samples 448 after interaction with hydrochloric acid, with the largest differences observed in carbonates (especially calcite).

FIG. 14 shows a graph 1498 of mass loss of rock samples 1248 based on interaction with a test fluid 429 according to certain example embodiments. Incorporating the description above with respect to FIGS. 1 through 13, the graph 1498 of FIG. 14 shows mass loss of two rock samples 1248 (rock sample 1248-1 and rock sample 1248-2) when combined with varying amounts of a test fluid 429 in the form of HCl. Specifically, the vertical axis shows mass loss as a percentage, and the horizontal axis shows the volume of 15% HCl added in mL. The rock samples 1248 in this case are from different field locations (e.g., different TVD, different well 420). Rock sample 1248-1 and rock sample 1248-2 show substantially the same amount of mass loss through the test fluid 429 reaching 4 mL. Beyond that volume of test fluid, rock sample 1248-2 does not experience any appreciable change in mass loss, whereas rock sample 1248-1 does experience appreciable mass loss. The results shown in this graph 1498 illustrate that use of example embodiments allows for custom field mineralogy evaluation to tailor acid/chemical usage.

Use of example embodiments may be used in a number of different situations and/or to achieve a number of different objectives. Example embodiments may be used to characterize, forecast, and/or improve reservoir and well performance and reduce water production shale asset developments. In addition, or in the alternative, example embodiments may be used to optimize fracturing and completion strategies to improve returns. The following table lists some examples of how example embodiments may be used:

| Sample Analysis Method | Subject of Investigation | Objective |
|---|---|---|
| Drilling mud return | Production section | Understand water sources at |
| filtrate analysis | waterflows; water source | the subsurface before |
| | identification | fracturing operations, predict |
| | | water sources during |
| | | fracturing operations, |
| | | understand impact of water- |
| | | related issues (e.g., subsurface |
| | | scale) on well performance, |
| | | and understand potential |
| | | changes in water sources |
| | | before and after fracturing |
| | | (e.g., as fracturing operations |
| | | may potentially connect the |
| | | lateral/producer with |
| | | additional water sources) |
| Rock-fluid/chemical | Combustible gas release from | Determine indications of |
| interaction tests | rocks (cutting/core samples) | hydrocarbon production |
| | | potential in shale matrixes by |
| | | hydraulic extraction or other |
| | | technologies depending on the |
| | | test design |
| Rock (drill cuttings, | Impact of chemical additives | Forecast and proactively |
| or core samples if | on release of Fe, Ba, Mn, Ca, | optimize fracturing |
| available) - fluid/ | Mg, and other elements, rock | fluid/chemical additives |
| chemical interaction | dissolution, and solid | program to manage impact of |
| tests | precipitation at the subsurface | fluid/chemicals related |
| | | production issues and improve |
| | | reservoir and production |
| | | performance and to improve |
| | | recovery of hydrocarbons; |
| | | optimize fracturing design; |
| | | and improve capital |
| | | efficiency; optimize capital |
| | | deployment (e.g., if a certain |
| | | part of the landing or lateral is |
| | | not known to show |
| | | hydrocarbon release, but the |
| | | lateral has been drilled, an |
| | | operator can make a conscious |
| | | choice not to stimulate a |
| | | certain part of the lateral). |

FIG. 15 shows a graph 1598 plotting measurements taken from rock samples 448. Incorporating the description above with respect to FIGS. 1 through 14, the graph 1598 of FIG. 15 indicates an amount of an ion concentration (in this case designated as “Y ion concentration”) along the vertical axis versus an amount of another ion concentration (in this case designated as “X ion concentration”) along the horizontal axis for samples of 5 different water sources WS (WS1, WS2, WS3, WS4, and WS5) as well as a sample taken from drilling mud return DM. X and Y in this case may be concentrations, ratios, stable isotope analysis results, and/or other determinations of a chemical element (e.g., Cl, Br, I, F, Li, Na, K, Ca, Mg, Sr, Ba, SO₄, etc.), including conservative ions (which may not be released or precipitated in the study system) and/or non-conservative ions (which may be released and/or precipitated in the study system). As an example, the analysis based on conservative elements such as Cl and Br in many cases may be used reliably for water source allocation. The allocation results, together with analysis of other ions, may be used to understand fluid compatibility and mineral precipitation.

A graph of this nature may arise using example embodiments when subsurface waterflow is encountered or suspected during drilling. In such cases, mud filtrate samples may be collected and analyzed to understand the potential source of the waterflow. In this case, the water chemistry data indicates the water phase from return mud (as indicated by DM in the graph 1598) is similar to WS1. Using example embodiments, water samples may be collected at different total depths (TDs), and testing these water samples may show variations in water sources along the lateral section of the development well.

Cross plots and data analysis based on ion concentrations, ion concentration ratios, stable isotope analysis results, some other variable, and/or any type of combination may be utilized for water source identification/allocation, rock-water interaction (e.g., mineral dissolution and transformation), and/or drill in fluid-formation fluid incompatibility/mineral precipitation investigation. Some of the findings from this work may be used to better interpret well logging test results, e.g., for water saturation determination and other purposes.

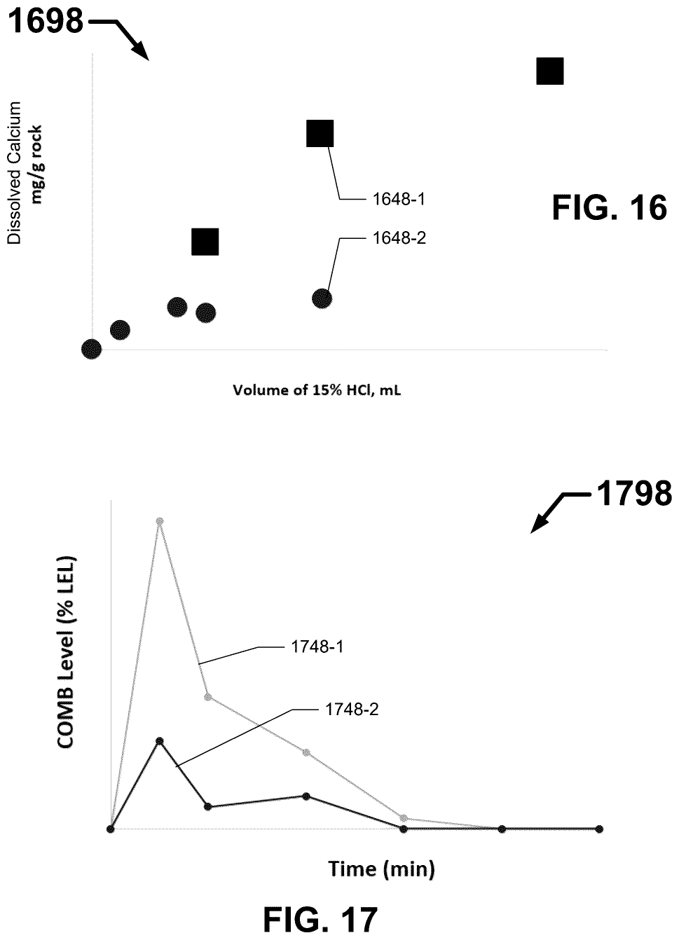

FIGS. 16 through 19 are part of an example case study that may result from using example embodiments. Incorporating the description above with respect to FIGS. 1 through 15, in this example case study, rock samples 1648 from two different wells (e.g., wells 420) of the same field are obtained and tested. FIG. 16 shows a graph 1698 of dissolved calcium (in mg/g of each rock sample 1648) along the vertical axis versus the volume of a test fluid 429 in the form of 15% HCl (in mL) along the horizontal axis. The graph 1698 shows that rock sample 1648-1 has a higher amount of dissolved calcium relative to the amount of dissolved calcium in rock sample 1648-2.

FIG. 17 shows a graph 1798 of gas (also known as headspace gas) in percent LEL along the vertical axis that results from combining the rock samples 1748 with a test fluid 429 in a vessel 461 over time in minutes along the horizontal axis. The graph 1798 shows that rock sample 1748-1 has a higher amount of headspace gas, particularly in the first few minutes, relative to the amount of headspace gas associated with rock sample 1748-2.

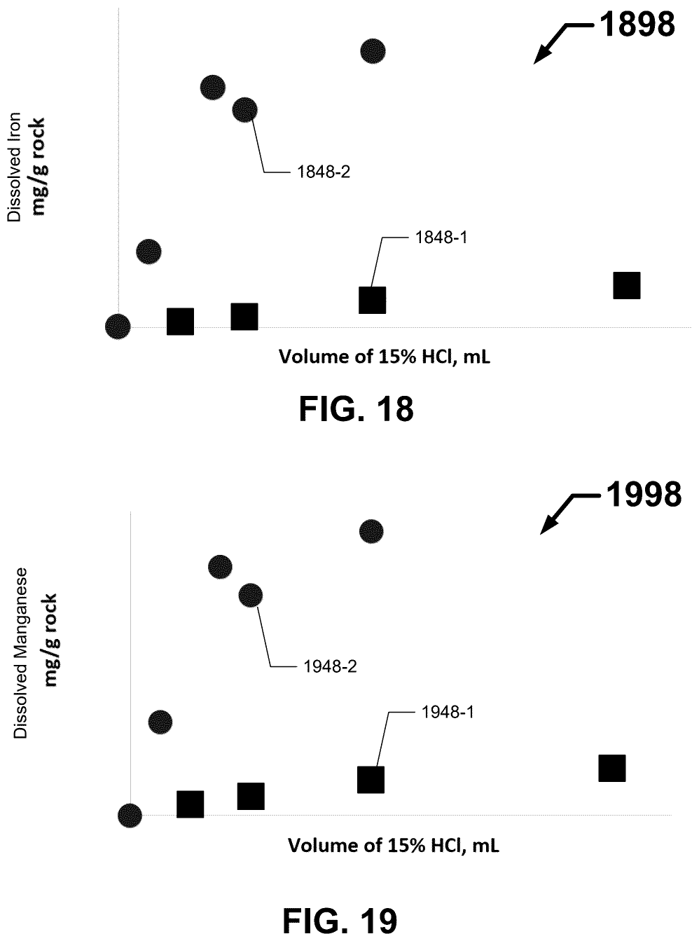

FIG. 18 shows a graph 1898 of dissolved iron (in mg/g of each rock sample 1848) along the vertical axis versus the volume of a test fluid 429 in the form of 15% HCl (in mL) along the horizontal axis. The graph 1898 shows that rock sample 1848-2 has a higher amount of dissolved iron relative to the amount of dissolved iron in rock sample 1848-1. FIG. 19 shows a graph 1998 of dissolved manganese (in mg/g of each rock sample 1948) along the vertical axis versus the volume of a test fluid 429 in the form of 15% HCl (in mL) along the horizontal axis. The graph 1998 shows that rock sample 1948-2 has a higher amount of dissolved manganese relative to the amount of dissolved manganese in rock sample 1948-1.

To complement the graphs of FIGS. 16 through 19, the following table summarizes some of the findings of the example case study using example embodiments. The conclusion of the example case study is that the well 420-1 from which the rock samples 1848-1 are collected is more likely to have better performance than the well 420-2 from which the rock samples 1848-2 are collected.

| Required tests | | Assessment | |
|---|---|---|---|
| and analysis | Key factors | Well 420-1 | Well 420-2 |
| Rock-fluid | Ca/Mg released to | Higher calcium, | Lower calcium, likely |
| interaction | aqueous phase from | likely higher level of | lower level of |
| | rock-acid interaction | stimulation by acid | stimulation by acid |
| | | treatment | treatment |
| Rock-fluid | Fe/Mn/Ba etc. | Lower Fe, Mn; Lower | Higher Fe, Mn; FeS |
| interaction | released to aqueous | potential for Fe- | concern, higher risk |
| | phase from rock-acid | containing deposits | of deposit formation; |
| | interaction | | Fe control may be |
| | | | required |
| Rock-fluid | Released combustible | Higher | Lower |
| interaction | gas (CH₄, C₂H₆, C₃H₈, | | |
| | . . . ) | | |
| Tests/analysis/data | Water chemistry | Results do not suggest | Moveable water |
| interpretation based | parameters (Cl, SO₄, | significant presence | sources from non- |
| on filtrate from | Ca, Na, K, Mg, Ca, Sr, | of subsurface water | target formation or |
| returned drilling mud | Ba, Br, NO₃, I, etc.) | sources in returned | water channeling |
| | | drilling mud | from injection wells |
| | | | are suspected |

| | | |
|---|---|---|
| Pre-completion | Production | Well (e.g., 420-1) is predicted to have better |
| assessment | performance/EUR, | well performance than well (e.g., 420-2). Acid |
| | fluid-related | treatment (e.g., HCl) may have more positive |
| | production risks | impact on production performance for well |
| | | 420-1 than for well 420-2. |

Example embodiments may be used to establish correlations and/or collaborations of a current well 420 with one or more existing wells 420. For instance, example embodiments may be used to identify key factors and establish a workflow with key indexes and “calibration” envelope/algorithm to assess, forecast, and/or improve TRU reservoir and individual well performance utilizing test results from rock and fluid samples collected during drilling stages for both ongoing and future developments. A formula, model, and/or other form of algorithm 533 may or may not be developed and/or utilized in this part of the process.

When using existing wells 420, different types of data may be applied to a subject well 420 using example embodiments. Examples of such data may include, but are not limited to, production data, workover jobs, field observations, chemical treatments, mud gas data, observations during drilling, and external water invasion. In addition, or in the alternative, various tests and factors may be utilized, including but not limited to rock-fluid interaction tests with available cutting and/or core samples, mud gas analysis, subsurface chemicals and fluid chemistry data, geoscience factors (e.g., structural configuration, lithology, stratigraphy methods, gross thickness, net-to-gross ratio, net pay, porosity, saturation, permeability, heterogeneity), engineering factors (e.g., reservoir depth, pressure, temperature, fluid properties, recovery mechanisms, fluid mobilities, fluid distribution, well productivity), and operational factors (e.g., water depth, well type, completion, spacing, facility type and constraints, artificial lift, pattern type and spacing, injector/producer ratio).

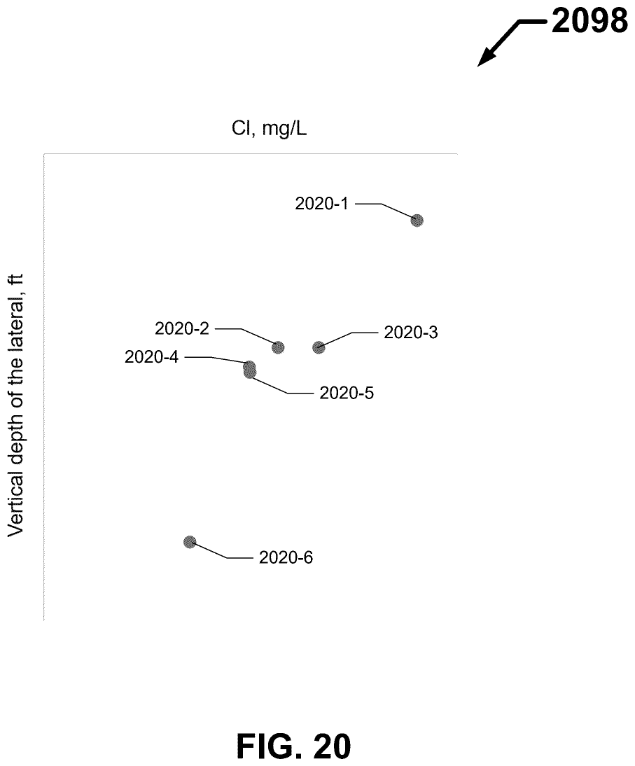

The field operation plan generated using example embodiments may include one or more recommendations for an operational fluid, which may be in the form of chemical additives (e.g., function, product name, concentration, volume), fracturing fluid (also sometimes called fracturing water), reservoir fluids (e.g., water, hydrocarbons)). FIG. 20 shows a graph 2098 of a volume of a recommended operation fluid 437 for multiple wells 2020 according to certain example embodiments. Incorporating the description above with respect to FIGS. 1 through 19, the graph 2098 of FIG. 20 plots the vertical depth (in feet) of the lateral for each of 6 wells 2020 (well 2020-1, well 2020-2, well 2020-3, well 2020-4, well 2020-5, and well 2020-6) along the vertical axis and the volume of chlorine (in mg/L) for the recommended operational fluid 437 along the horizontal axis.

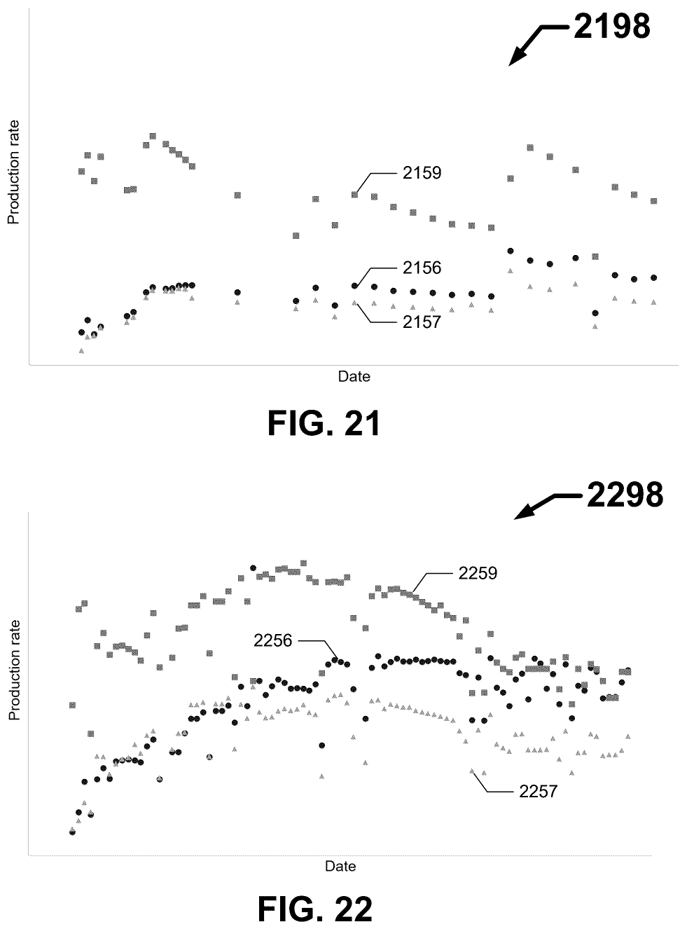

Example embodiments may consider field production and acid (or other form of operation fluid) usage data. For example, the graphs of FIGS. 21 through 23 show that, in this case, well production performance is negatively correlated with the 15% HCl acid usage (volume per foot) in completion. Incorporating the description above with respect to FIGS. 1 through 20, the graph 2198 of FIG. 21 shows a case where the operation fluid 437 includes 21 gallons of 15% HCl per foot for a particular well 2320-1. The horizontal axis of the graph 2198 is time, and the vertical axis of the graph 2198 is a rate (MCF per day for gas 2156 (shown as circles in the graph 2198), barrels per day for oil 2157 (shown as triangles in the graph 2198), and barrels per day for water 2159 (shown as squares in the graph 2198)).

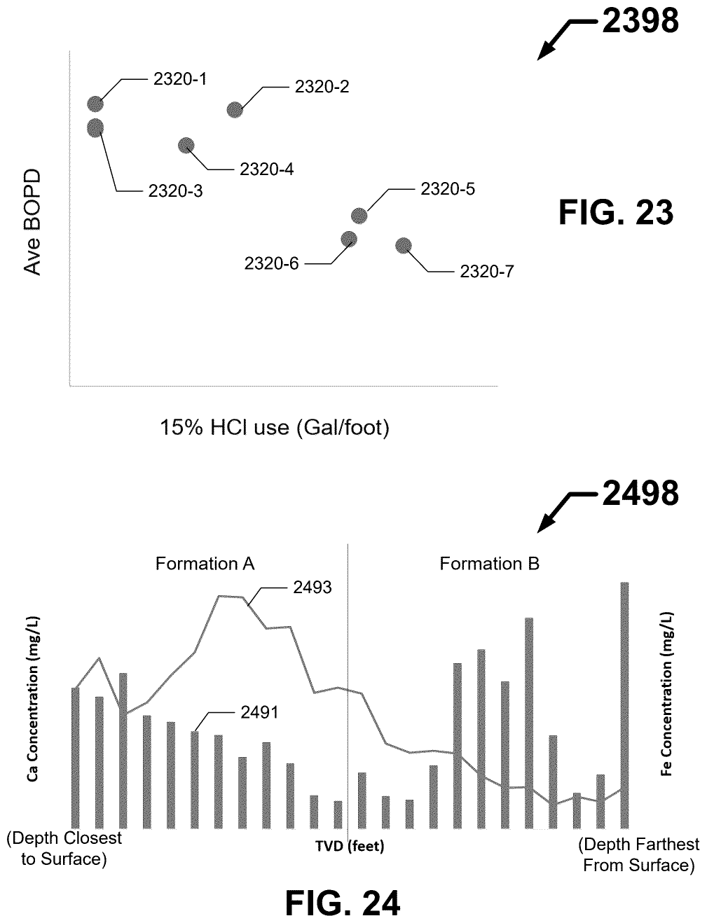

The graph 2298 of FIG. 22 shows a case where the operation fluid 437 includes 10 gallons of 15% HCl per foot for a particular well 2320-2 that differs from the well 2320-1 used in the graph 2198 of FIG. 21. The horizontal axis of the graph 2298 is time, and the vertical axis of the graph 2298 is a rate (MCF per day for gas 2256 (shown as circles in the graph 2298), barrels per day for oil 2257 (shown as triangles in the graph 2298), and barrels per day for water 2259 (shown as squares in the graph 2298)). The graph 2398 of FIG. 23 shows a plot for seven wells 2320 (well 2320-1, well 2320-2, well 2320-3, well 2320-4, well 2320-5, well 2320-6, and well 2320-7). Specifically, the vertical axis of the graph 2398 of FIG. 23 is in barrels of oil per day, and the horizontal axis of the graph 2398 of FIG. 23 is where the operation fluid 437 includes 15% HCl (expressed in gallons per foot).

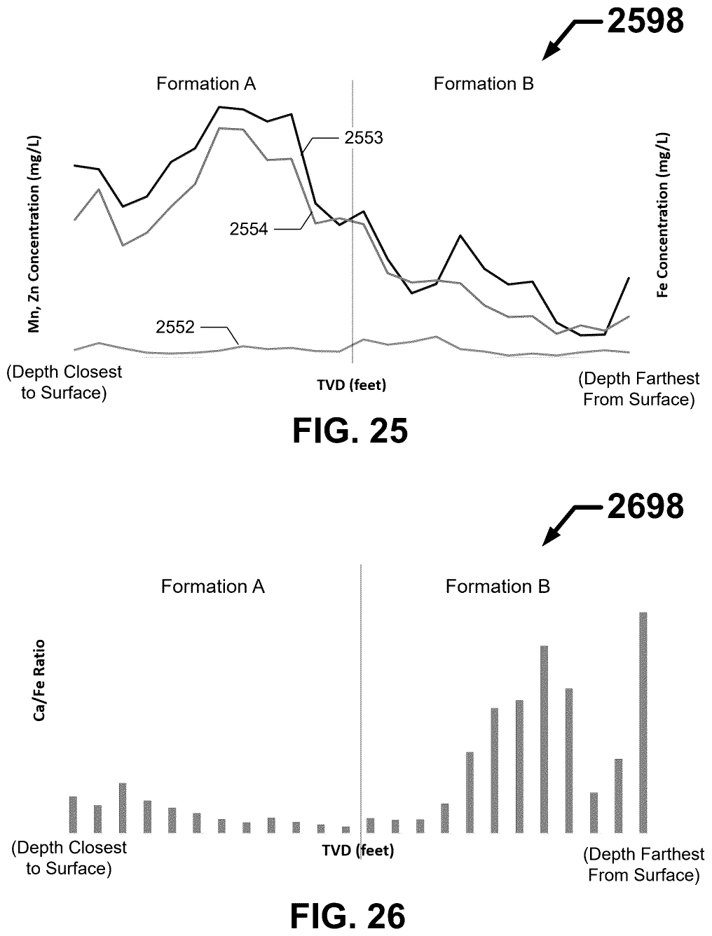

FIG. 24 shows a graph 2498 of changes in calcium (shown by plot 2491) and iron (shown by plot 2493) in rock samples 448 over a range of depths that span two formations (formation A and formation B) according to certain example embodiments. FIG. 25 shows a graph 2598 of changes in manganese (shown by plot 2553), iron (shown by plot 2554), and zinc (shown by plot 2552) in rock samples 448 over a range of depths that span two formations (formation A and formation B) according to certain example embodiments. FIG. 26 shows a graph 2698 of calcium to iron ratios for rock samples 448 over a range of depths that span two formations (formation A and formation B) according to certain example embodiments. Incorporating the description above with respect to FIGS. 1 through 23, put another way, elemental analysis results are shown in FIGS. 24 and 25 to demonstrate typical experiment output. Changes in Ca, Fe, Mn, and Zn concentration were observed, and the Ca/Fe ratio, which could be used as an indicator to different formation solids, is displayed in FIG. 26. The graphs of FIGS. 24 through 26 show the results from one set of rock samples 448 collected in one part (formation A) of the subterranean formation, and also from a second set of rock samples 448 collected in another part (formation B) of the subterranean formation.

Specifically, the graph 2498 of FIG. 24 shows a plot 2491 of calcium concentration (in mg/L) along the left vertical axis versus TVD (in feet) and another plot 2493 of iron concentration (in mg/L) along the right vertical axis versus TVD (in feet). The graph 2598 of FIG. 25 shows a plot 2552 of zinc concentration (in mg/L) and a plot 2553 of manganese concentration (in mg/L) along the left vertical axis versus TVD (in feet) and another plot 2554 of iron concentration (in mg/L) along the right vertical axis versus TVD (in feet). The graph 2698 of FIG. 26 shows a plot of the calcium-to-iron ratio (unitless) along the vertical axis versus TVD (in feet).

FIG. 27 shows part of another analytic system 2750 according to certain example embodiments. Referring to the description with respect to FIGS. 1 through 26 above, the part of the analytic system 2750 shown in FIG. 27 includes parts of a single testing apparatus 2770. Specifically, the testing apparatus 2770 of FIG. 27 includes a vessel 2761 in the form of a conical flask and a sensor device 2760 in the form of a gas detector with a probe 2763 that extends through a stopper 2764 at the top end of the vessel 2761 and terminates inside the vessel 2761. Disposed within the vessel 2761 is a rock sample 2748. Combined with the rock sample 2748 in the vessel 2761 is a test fluid 2729 in the form of a stimulation agent (e.g., an acid) and/or water. Some of the test fluid 2729 may be added by a reagent source 2768, which provides a reagent (e.g., HCl, HNO₃, acetic acid, CO₂ saturated acidic water, calcium carbonate) into the vessel 2761 through a tube that extends through a stopper 2764 at the top end of the vessel 2761 and terminates inside the vessel 2761.

In some cases, hydrocarbons 2766 (a form of subterranean resource 111) may be retained in the rock samples 2748 (e.g., unpreserved shale cuttings, core samples). Measuring parameters associated with hydrocarbons 2766 released from the interaction between the rock samples 2748 and the test fluid 2729 may provide a more accurate indication (sometimes called an index value herein) of the potential amount of subterranean resources that may be recovered from the wellbore using hydraulic fracturing technology. The larger the rock samples 2748 (e.g., in terms of the number of rock samples 2748, in terms of the size of each rock sample 2748), the stronger the indication of the potential amount of subterranean resources that may be recovered from the wellbore based on the hydrocarbons 2766 released from the interaction of the rock samples 2748 and the test fluid 2729 in the vessel 2761.

In some cases, the interaction between the rock sample 2748 and the test fluid 2729 yields a hydrocarbon 2766 (e.g., a gas, a liquid) that is released from the rock sample 2748, rises through the test fluid 2729 (or is otherwise separated from the test fluid 2729), and collects above the test fluid 2729 within the vessel 2761. The sensor device 2760 is then able to measure one or more parameters associated with the hydrocarbons 2766. For example, the sensor device 2760 may be configured to measure one or more gaseous parameters (e.g., volume of combustible gas, O₂, chemical composition, mass) associated with the hydrocarbons 2766. As another example, in terms of subterranean resources 2711 released from the rock sample 2748 in liquid form, the sensor device 2760 (e.g., in the form of an oil in water analyzer) may be used to measure the chemical composition, mass, and/or volume of hydrocarbons 2766 in liquid form extracted from the rock sample 2748.

In some cases, when the rock samples 2748 are relatively large, interaction of the rock samples 2748 with the test fluid 2729 may allow the sensor device 2760 to test hydrocarbons 2766 in the form of oil extracted from the rock samples 2748. In such a case, the sensor device 2760 may measure a fingerprinting profile of the oil. Using this approach, example embodiments of the analytic system 2750 may be used to estimate gas/water/oil ratios from the hydrocarbons 2766 released from the rock samples 2748 to generate a production forecast (e.g., for an amount of hydrocarbons that may potentially be recovered by hydraulic fracturing technology) for the subterranean formation (e.g., subterranean formation 110) from which the rock samples 2748 are obtained because of the indication from the rock-fluid interaction with respect to production performance.

In addition to generating a production forecast, example embodiments may be used for other predictive and planning purposes. For example, analysis of hydrocarbons 2766 released from the rock samples 2748 (e.g., cuttings, core samples) that are taken from the substantially vertical section of a wellbore may be used for determining an optimal landing location in a well pad and/or placement of a future well. As another example, analysis of hydrocarbons 2766 released from rock samples 2748 (e.g., cuttings, core samples) that are taken from the substantially horizontal section of a wellbore may be used for determining an optimal completion plan for the well. Example embodiments may be used for optimizing current and future wells in shale and tight formations.

The sensor device 2760 may be configured to measure one or more of a number of parameters associated with the hydrocarbons 2766 released from the rock samples 2748 after the rock samples 2748 interact with the test fluid 2729 in the vessel 2761. For example, the sensor device 2760 may be configured to measure an amount of gas and/or liquid hydrocarbons 2766 released from the rock samples 2748 after the rock samples 2748 interact with the test fluid 2729 in the vessel 2761. As another example, the sensor device 2760 may be configured to measure the composition of gas hydrocarbons 2766 released from the interaction of the rock samples 2748 and the test fluid 2729.

As another example, the sensor device 2760 may be configured to measure the mass of the hydrocarbons 2766 released from the interaction of the rock samples 2748 and the test fluid 2729. Such data may help with projecting production capability of the well. As another example, the sensor device 2760 may be configured to measure the molecules per volume of the hydrocarbons 2766 released from the interaction of the rock samples 2748 and the test fluid 2729. Such data may help with projecting production capability of the well. As another example, the sensor device 2760 may be configured to measure the mass of the rock samples 2748 after the interaction of the rock samples 2748 and the test fluid 2729. Such data may help with projecting production capability of the well.

As another example, the sensor device 2760 may be configured to measure the chemistry signature of the hydrocarbons 2766 (e.g., in gas form, in liquid form) released from the interaction of the rock samples 2748 and the test fluid 2729. Such data may help with assessing the production allocation of the well, as when correlated with samples of produced gas and/or oil during production of the well. As another example, the sensor device 2760 may be configured to measure the chemistry signature of the test fluid 2729 (e.g., formation water) that remains after the hydrocarbons 2766 are released from the interaction of the rock samples 2748 and the test fluid 2729. Such data may help with assessing the production allocation of the well, as when correlated with samples of produced water during production of the well.

In some cases, the sensor device 2760 may be configured to measure one or more parameters associated with Cl in formation water. In such a case, the test fluid 2729 may include a reagent from the reagent source 2768 where the reagent (e.g., HNO₃) does not include Cl. In addition, or in the alternative, the sensor device 2760 may be configured to measure one or more other parameters associated with formation water (part of the test fluid 2729). For example, HNO₃ may be used as a reagent from the reagent source 2768 to be part of the test fluid 2729 in order to the chemistry signature of the formation water after the hydrocarbons 2766 are extracted from the rock samples 2748. In such cases, HNO₃ with a water chemical tracer may be utilized to determine the chemistry signature of the formation water. The Cl concentration in the formation water and the volume of the formation water may be calculated based on the concentrations (e.g., measured by the sensor device 2760) of the water chemical tracer and the Cl. In certain example embodiments, a reagent of the reagent source 2768 may include a caustic solution that may adsorb CO₂ and/or H₂S within the vessel 2761.

The sensor device 2760 may take any of a number of forms for purposes of the analytic system 2750 of FIG. 27. For example, the sensor device 2760 may be or include a balance that is configured to measure mass. In such a case, the balance may be configured to measure the mass of the hydrocarbons 2766, the formation water, the rock samples 2748, and/or other components within the vessel 2761 in solid, liquid, and/or gas form. This mass balance may include or be attached to a data logger to measure the change (e.g., decrease) in mass over time (e.g., continuously, in discrete increments of time) and generate a graph (e.g., constant display with real time updates, on demand). In some cases, a filter (e.g., cotton wool) may be inserted in the neck of the vessel 2761 (e.g., in place of the stopper 2764) to allow gases (sometimes called headspace gases herein) to escape from the vessel 2761. With the balance, the mass of any solid, liquid, and/or gas in the vessel 2761 may be measured and recorded.

As another example, the sensor device 2760 may be or include a combustible gas detector and/or an explosimeter. In such cases, the hydrocarbon 2766 may be or include a flammable or explosive gas or vapor. Another example of a sensor device 1760 may be or include a catalytic bead sensor, which may monitor combustible gas concentrations through the temperature elevation of a filament. This temperature change is translated into a quantifiable indicator called the lower explosive limit (LEL). A potential drawback with using this approach is that while the sensor device 2760 may monitor gas concentrations, it may not identify the different gases or its contents. The identification and/or contents of a gas may be determined by a sensor device 2760 in the form of a gas chromatograph. When the hydrocarbon 2766 is or includes a liquid, examples of a sensor device 2760 may include, but are not limited to, an oil in water analyzer, which may determine an amount of liquid in the hydrocarbon 2766 and/or the mass measurement of the separated hydrocarbon phase.

In some cases, the analytic system 2750 may include a gas syringe 2771 to extract gases from the vessel 2761 for testing and/or measurement by a sensor device (e.g., sensor device 2760). In addition, or in the alternative, the analytic system 2750 may include components and/or equipment that may be used for a water displacement approach to collect and measure samples after the rock samples 2748 reacts with the test fluid 2729. As with the description above with respect to the analytic system 950 of FIG. 9, there may be a chemical reaction and evolution of mixed gases by combining the rock sample 2748 and the test fluid 2729 in the vessel 2761, which may be detected and broken down into relative percent components (e.g., O₂, H₂, CO₂, H₂S, CO, SO₂, and combustible gases such as hydrocarbons) using the sensor device 2760. The test fluid 2729 that is spent (e.g., in the form of formation water) may then be analyzed (e.g., using another sensor device 2760) for dissolved chemical species (e.g., cations, anions) by various water chemistry analyses.

In certain example embodiments, the analytic system 2750 of FIG. 27 may include a sonication device 2779. In such cases, the sonication device 2779 may be in communication with the vessel 2761. The sonication device 2779 may be configured to provide vibrations to the vessel 2761. When this occurs, the vibrations may cause any bubbles that form during interaction between the rock samples 2748 and the test fluid 2729 and/or during the release of the hydrocarbons 2766 from the rock samples 2748. By removing the bubbles, there may be a more consistent release of gas to be collected and/or measured by the sensor device 2760, leading to more consistent and reliable data.

While not shown in FIG. 27, the analytic system 2750 may also include a temperature regulating device configured to control the temperature of the contents (e.g., the rock samples 2748, the test fluid 2729, the hydrocarbons 2766) of the vessel 2761. Also, while not shown in FIG. 27, the analytic system 2750 may also include a pressure regulating device configured to control the pressure of the contents (e.g., the rock samples 2748, the test fluid 2729, the hydrocarbons 2766) of the vessel 2761. In some cases, the test environment of FIG. 27 may be combined with the test environment of FIG. 9 above. For example, the sensor device 2760 of FIG. 27 may be used to measure one or more parameters associated with hydrocarbons 2766, but also one or more parameters associated with other gases released from the interaction between the rock samples 2748 and the test fluid 2729. In such a case, the other gases may provide an indication of potential hydrocarbon production, optimal capital deployment (e.g., closing off a well, selective fracturing, smart completion), optimal fracturing fluid composition, optimal landing, etc.

When the test fluid 2729 is in the form of a strong reagent, the hydrocarbons 2766 released from the rock samples 2748 may be measured to determine the hydrocarbon production potential and/or hydrocarbon recovery. When the test fluid 2729 is in the form of a fracturing fluid (e.g., water, chemical additives), actual field conditions may be simulated. Such testing may take a relatively longer period of time to achieve meaningful results. Using either or both types of test fluid 2729, example embodiments may be used to optimize the fracturing fluid and/or field operations (e.g., shut in time between well completion and POP date), thereby optimizing the production performance of a well.

FIG. 28 shows a graph 2898 of an index value from headspace gas monitoring (as discussed above with respect to FIG. 27) according to certain example embodiments. Referring to the description with respect to FIGS. 1 through 27 above, the graph 2898 plots the index value along the vertical axis over time (in seconds) along the horizontal axis. The plot of the index values for well A shows a strong correlation with production performance. Since well A is an economic (producing) well, a decision may be made to enhance development and/or production of well A to capitalize on the remaining potential of well A. On the other hand, the plot for well B shows little if any production performance, current or future. Since well B is an uneconomic well, a decision may be made to stop development and/or production of well B to avoid further uneconomic investment in well B.

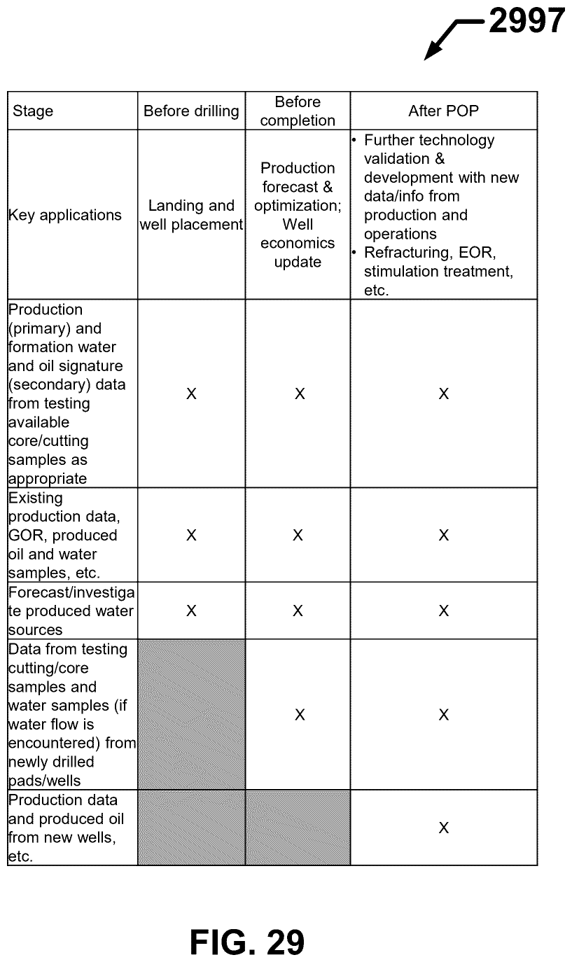

FIG. 29 shows a table 2997 that highlights the capabilities of utilizing an example analytic system over time according to certain example embodiments. As discussed above, the use of data collected using example embodiments may be used for different stages along the life of a well. For example, before drilling a well, example embodiments using data associated with hydrocarbons released from rock samples of an existing well that interact with test fluids may be used to help determine the landing and placement of the well to be drilled. For example, a production analysis using data associated with hydrocarbons released from rock samples of an existing well that interact with test fluids may identify where to land. As another example, a formation water analysis using data associated with hydrocarbons released from rock samples of an existing well that interact with test fluids may identify information about production allocation and/or drainage height of a prospective well. The associated data may also be used for estimating production data, GOR, produced oil and water samples, and other information associated with a prospective well. The associated data may further be used to forecast and/or investigate produced water sources associated with a prospective well.

Referring to the description of FIGS. 1 through 28 above, the table 2997 of FIG. 29 shows that, before completion of an existing well, data associated with hydrocarbons released from rock samples from that well that interact with test fluids may be used to help with a production forecast, production optimization, and/or an economic forecast for that well. For example, a production analysis and formation water analysis using data associated with hydrocarbons released from rock samples of the well that interact with test fluids may help generate a production forecast, production optimization, and/or an economic forecast for that well before completion of the well. As another example, a formation water analysis using data associated with hydrocarbons released from rock samples from the well that interact with test fluids may be used for estimating production data, GOR, produced oil and water samples, and other information associated with the well before completion of the well. The data associated with hydrocarbons released from rock samples of the well that interact with test fluids may further be used to forecast and/or investigate produced water sources associated with the well before completion of the well. The data associated with hydrocarbons released from rock samples of the well that interact with test fluids for the well may also include data associated with hydrocarbons released from rock samples from one or more other adjacent wells that interact with test fluids.

The table 2997 of FIG. 29 also shows that, after a well is put on production (POP), data associated with hydrocarbons released from rock samples from that well that interact with test fluids may be used to help provide technology validation and development with new data and/or information from production and operations for that well. Also, after a well is POP, data associated with hydrocarbons released from rock samples from that well that interact with test fluids may be used to help serve as a reference for refracturing, EOR (e.g., chemical treatment, surfactant treatment, polymer treatment), simulation treatment, shutting in the well for a period of time, and/or other forms of enhanced operations.

A production analysis and formation water analysis using data associated with hydrocarbons released from rock samples of the well that interact with test fluids may help generate a production forecast, production optimization, and/or an economic forecast for that well after being POP. As another example, a formation water analysis using data associated with hydrocarbons released from rock samples from the well that interact with test fluids may be used for estimating production data, GOR, produced oil and water samples, and other information associated with the well after being POP. The associated data may further be used to forecast and/or investigate produced water sources associated with the well after being POP. The data associated with hydrocarbons released from rock samples of the well that interact with test fluids for the well may also include data associated with hydrocarbons released from rock samples from one or more other adjacent wells that interact with test fluids. The data associated with hydrocarbons released from rock samples of the well that interact with test fluids for the well may also include production data and data associated with produced oil from one or more other adjacent wells.

Data associated with hydrocarbons released from rock samples of the well that interact with test fluids may be organized (e.g., in a table or series of tables) and used in conjunction with other data and/or adjacent wells. An example of such other data may include subsurface chemicals and fluid chemistry data, which may include but are not limited to chemical additives (e.g., function, product name, concentration, volume), fracturing fluid, and reservoir fluids (e.g., formation water, hydrocarbons). Another example of such other data may include geoscience data, which may include but is not limited to structural configuration, lithology, stratigraphy methods, gross thickness, net-to-gross ratio, net pay, porosity, saturation, permeability, and heterogeneity. Yet another example of such other data may include engineering data, which may include but is not limited to reservoir depth, pressure, temperature, fluid properties, recovery mechanisms, fluid mobilities, fluid distribution, and well productivity. Still another example of such other data may include operational data, which may include but is not limited to water depth, well type, completion, spacing, facility type and constraints, artificial lift, pattern type and spacing, and injector/producer ratio.

Example embodiments may include mechanisms for validating measurements made by sensor devices and/or outputs from models and other algorithms regarding data associated with hydrocarbons released from rock samples of the well that interact with test fluids. Example embodiments may additionally or alternatively include mechanisms for database development and analytics. Example embodiments may additionally or alternatively include mechanisms for field case studies and well performance lookback. Example embodiments may additionally or alternatively include mechanisms for promoting best practices on the application of relevant technologies contemplated herein.

When a sensor device (e.g., sensor device 2760) is measuring one or more parameters (e.g., mol. %, molecular weight, Wt. %, specific gravity, L.V. %, pounds per gallon (in air), pounds per gallon (in vacuum), API gravity, cubic feet vapor per gallon) associated with a gas, examples of such a gas may include, but are not limited to, hydrogen, oxygen, nitrogen, methane, carbon dioxide, ethane, propane, iso-butane, n-butane, iso-pentane, n-pentane, i-hexanes, n-hexane, 2,2,4-trimethylpentane, benzene, heptanes, toluene, octanes, ethylbenzene, xylenes, nonanes, and decanes.

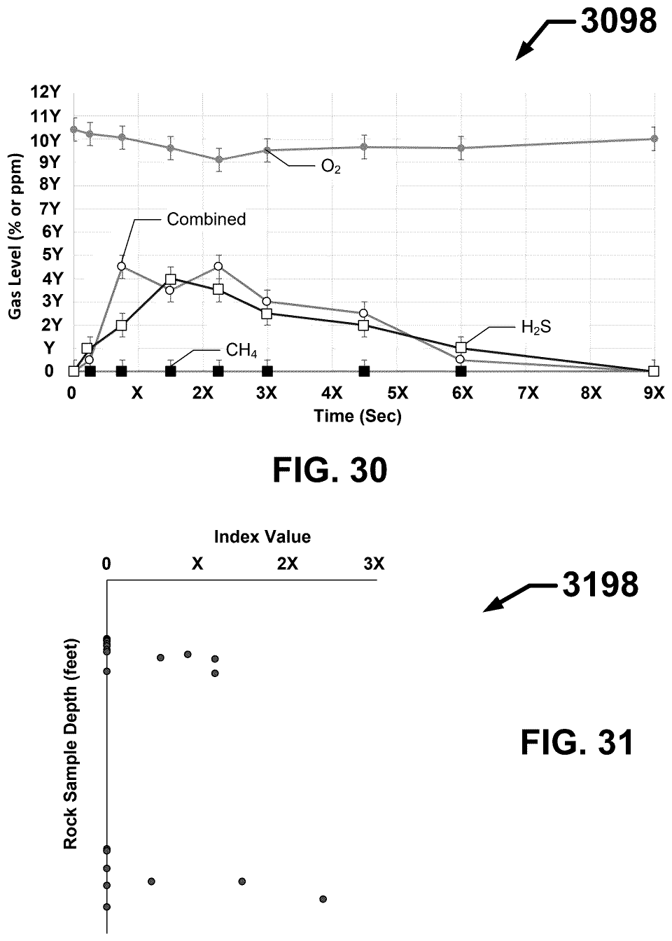

FIG. 30 shows another graph 3098 of headspace gas monitoring according to certain example embodiments. Referring to the description with respect to FIGS. 1 through 29 above, the graph 3098 plots a gas level (either as a percentage or in ppm) along the vertical axis over time (in seconds) along the horizontal axis. The headspace gas is collected from a vessel (e.g., vessel 2761) after rock samples (e.g., rock samples 2748) interact with a test fluid (e.g., test fluid 2729) (e.g., an acid). Specifically, the graph 3098 shows four plots. One plot is for O₂ (with plot points as closed circles) as a percentage LEL. Another plot is for the combined gas (with plot points as open circles) as a percentage. Another plot is for H₂S (with plot points as open squares) in ppm. The final plot is for CH₄ (with plot points as closed squares) as a percentage.

FIG. 31 shows another graph 3198 of hydrocarbon production potential from headspace gas monitoring according to certain example embodiments. Referring to the description with respect to FIGS. 1 through 30 above, the graph 3198 plots the rock sample depth (in feet) along the vertical axis versus an index value associated with potential hydrocarbon production based on hydrocarbons released from rock samples of the well that interact with test fluids along the horizontal axis. The rock samples in this case are collected within two relatively small ranges. For the rock samples collected from the upper range of depths (closer to the surface), the values associated with hydrocarbons released from rock samples of the well that interact with test fluids are generally lower than the values for the rock samples collected from the lower range of depths.

FIG. 32 shows part of yet another analytic system 3250 according to certain example embodiments. Referring to the description with respect to FIGS. 1 through 31 above, the part of the analytic system 3250 shown in FIG. 32 includes a testing apparatus 3270 having three vessels (vessel 3261-1, vessel 3261-2, and vessel 3261-3) that are interconnected. The vessel 3261-1 of the testing apparatus 1270 has a cylindrical form with tubing 3278-1 that extends through a stopper 3264 at the top end of the vessel 3261-1 and terminates inside the vessel 3261-1. Disposed within the vessel 3261-1 is a rock sample 3248. Combined with the rock sample 3248 in the vessel 3261-1 is a test fluid 3229 in the form of a stimulation agent (e.g., an acid) and/or water.

In some cases, hydrocarbons 3266 (a type of subterranean resource 111) may be retained in the rock samples 3248 (e.g., unpreserved shale cuttings, core samples) when the rock samples 3248 are placed in the vessel 3261-1. Measuring parameters associated with the hydrocarbons 3266 released from the interaction between the rock samples 3248 and the test fluid 3229 within the vessel 3261-1 may provide a more accurate indication of the potential amount of subterranean resources that may be recovered from the wellbore using hydraulic fracturing technology.

The larger the rock samples 3248 (e.g., in terms of the number of rock samples 3248, in terms of the size of each rock sample 3248), the stronger the indication of the potential amount of subterranean resources that may be recovered from the wellbore based on the hydrocarbons 3266 released from the interaction of the rock samples 3248 and the test fluid 3229 in the vessel 3261-1. In this case, the hydrocarbons 3266 released from the interaction of the rock samples 3248 and the test fluid 3229 are in gaseous form and bubble upward through the test fluid 3229 within the vessel 3261-1 and collect in the space within the vessel 3261-1 above the test fluid 3229. When enough of the gaseous hydrocarbons 3266 collect in the head space of the vessel 3261-1, the hydrocarbons 3266 collect in and flow through the tubing 3278-1, the other end of which terminates within the vessel 3261-2 of the testing apparatus 3270.

The vessel 3261-2 of the testing apparatus 3270 has water 3269 (or some other fluid) that fills some, but not all, of the space within the vessel 3261-2. In certain example embodiments, the volume of the vessel 3261-2 and the volume of the water 3269 is known before the hydrocarbons 3266 are released from the interaction of the rock samples 3248 and the test fluid 3229 in the vessel 3261-1 of the testing apparatus 3270. The top end of the vessel 3261-2 of the testing apparatus 3270 has an aperture through which part of the tubing 3278-1 traverses. The distal end of the tubing 3278-1 is positioned within the headspace in the vessel 3261-2 of the testing apparatus 3270.

As more of the hydrocarbons 3266 enter the headspace of the vessel 3261-2 of the testing apparatus 3270 through the tubing 3278-1, pressure builds within the headspace against the water 3269. As the pressure in the headspace of the vessel 3261-2 continues to build, the water 3269 is forced into tubing 3278-2, one end of which is positioned in the water 3269 within the vessel 3261-2. The tubing 3278-2 traverses an aperture in the top of the vessel 3261-2 and is sealed against the vessel 3261-2 by a stopper 3264. The other end of the tubing 3278-2 traverses an aperture in the top of the vessel 3261-3 of testing apparatus 3270 and is sealed against the vessel 3261-3 by another stopper 3264. The distal end of the tubing 3278-2 is positioned toward the top of the vessel 3261-3 in a headspace.

The water 3269 forced into the tubing 3278-2 by the pressure induced due to the accumulation of hydrocarbons 3266 in the headspace of the vessel 3261-2 of the testing apparatus 3270 flows through the tubing 3278-2 into vessel 3261-3 of the testing apparatus 3270, where the water 3269 collects and accumulates. Throughout the experiment and/or at the end of the experiment, the amount (e.g., in terms of volume, in terms of weight) of water 3269 that results in the vessel 3261-3 of the testing apparatus 3270 is measured (e.g., using one or more sensor devices (e.g., sensor device 460)). The amount of water 3269 that accumulates in the vessel 3261-3 may correlate to that amount of subterranean resources that may be produced from the part of the subterranean formation from which the rock samples 3248 originate.

As with the analytic system 2750 of FIG. 27, in addition to generating a production forecast, example embodiments may be used for other predictive and planning purposes. For example, measuring the amount of water 3269 displaced by the hydrocarbons 3266 released from the rock samples 3248 (e.g., cuttings, core samples) that are taken from the substantially vertical section of a wellbore may be used for determining an optimal landing location in a well pad and/or placement of a future well. As another example, measuring the amount of water 3269 displaced by the hydrocarbons 3266 released from rock samples 3248 (e.g., cuttings, core samples) that are taken from the substantially horizontal section of a wellbore may be used for determining an optimal completion plan for the well.

In certain example embodiments, the analytic system 3250 of FIG. 32 may include one or more of a number of other features and/or components (e.g., a sonication device, a temperature regulating device, a pressure regulating device) to help control some or all of the testing apparatus 3270 during the process discussed above. For example, such a feature and/or component may be applied to the vessel 3261-1 during interaction between the rock samples 3248 and the test fluid 3229 and/or during the release of the hydrocarbons 3266 from the rock samples 3248.

When the test fluid 3229 is in the form of a strong reagent, the hydrocarbons 3266 released from the rock samples 3248 may be measured to determine the hydrocarbon production potential and/or hydrocarbon recovery. When the test fluid 3229 is in the form of a fracturing fluid (e.g., water, chemical additives), actual field conditions may be simulated. Such testing may take a relatively longer period of time to achieve meaningful results. Using either or both types of test fluid 3229, example embodiments may be used to optimize the fracturing fluid and/or field operations (e.g., shut in time between well completion and POP date), thereby optimizing the production performance of a well.

FIG. 33 shows a table 3397 that highlights the capabilities of utilizing an example analytic system over time according to certain example embodiments. Referring to the description with respect to FIGS. 1 through 32 above, the table 3397 of FIG. 33 shows the status of 11 wells that are drilled through the same subterranean formation. Wells 1 and 2 are currently on production (POP), and so have samples taken from the heel, mid, and toe of their lateral sections. As a result of measuring one or more parameters associated with hydrocarbons that are released from an interaction between the rock samples and one or more test fluids, some index value is assigned to the results. An overall average index value is also determined based on the measurements of the hydrocarbons released from an interaction between the rock samples and one or more test fluids the along the entire lateral of each wellbore.

Wells 3, 4, and 8 through 14 are drilled but uncompleted wells (DUCs), which means that they have been drilled but fracturing operations have yet to be performed. Example embodiments may be used to determine which of these wells 3, 4, and 8 through 14 are worth pursuing and which should be closed off (e.g., safely abandoned). For example, as shown in the table, overall index values are assigned to each of wells 3, 4, and 8 through 14. These overall index values may be based, at least in part, on measuring one or more parameters associated with hydrocarbons that are released from an interaction between the rock samples from those wells and one or more test fluids.

As the table 3397 of FIG. 33 shows, the index value for wells 3 is 15, and the index value for well 4 is zero or falls below a minimum threshold value. As a result, a user (e.g., user 451) may determine that wells 3 and 4 should be closed off (e.g., safely abandoned) because there is not a sufficient amount of hydrocarbons that can be produced from those wells to justify the time and expense in performing fracturing operations. By contrast, the index values for wells 8 through 14 are relatively high. As a result, a user (e.g., user 451) may determine that fracturing operations should be performed on some or all of wells 8 through 14 because there is a sufficient amount of hydrocarbons that can be produced from those wells to justify the time and expense.

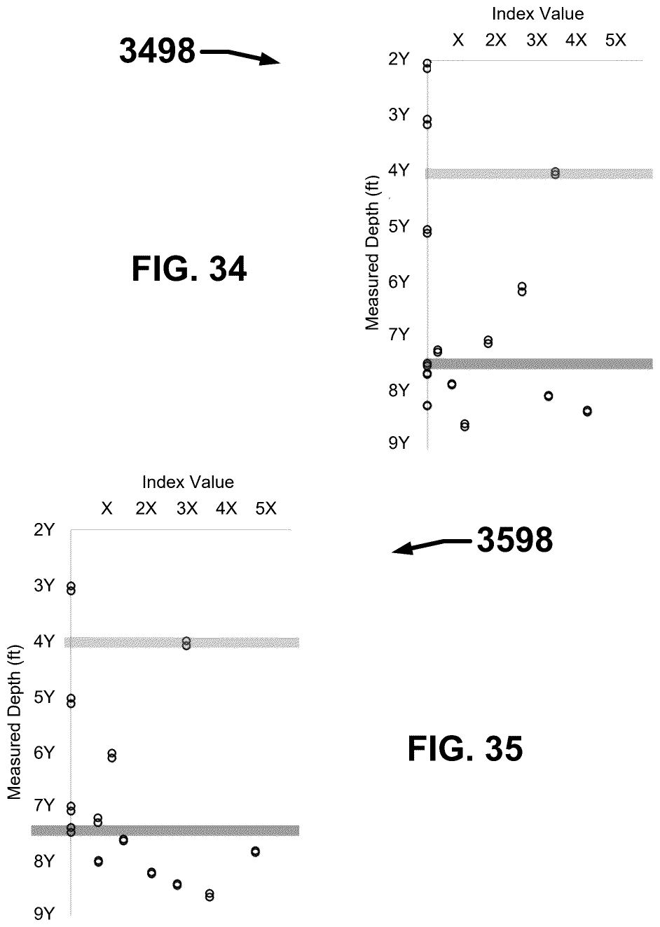

FIGS. 34 and 35 show graphs of hydrocarbon production potential for adjacent wellbores based on testing rock samples using the analytic system 3250 of FIG. 32 according to certain example embodiments. Referring to the description with respect to FIGS. 1 through 33 above, each of the graph 3498 of FIG. 34 and the graph 3598 of FIG. 35 show plots of the rock sample depth (in feet) for the adjacent wellbores along the vertical axis versus an index quantifying hydrocarbon production potential of the wellbores using the analytic system 3250 of FIG. 32 along the horizontal axis. In this case, each well shows a strong potential for hydrocarbon production at a depth of approximately 4 Y and at depths between approximately 8 Y and 9 Y. To the extent that the two wells traverse one or more common layers (e.g., around 4 Y depth, around 8.5 Y depth) of the subterranean formation, a user can produce at these depths in these wells with increased confidence of increased hydrocarbon production and that enhanced production techniques (e.g., fracturing) may help yield these improved results.

For example, using the information revealed in the graph 3498 of FIG. 34 and the graph 3598 of FIG. 35, a user (e.g., user 451) may determine that a substantially horizontal section of the wellbore used for the graph 3498 or the graph 3598, or an entirely new wellbore, should be landed at a depth of approximately 4 Y (one layer of the subterranean formation) or at a depth between approximately 8 Y and 9 Y (another layer of the subterranean formation), where the substantially horizontal section of the wellbore is kicked off from a substantially vertical section of the wellbore.

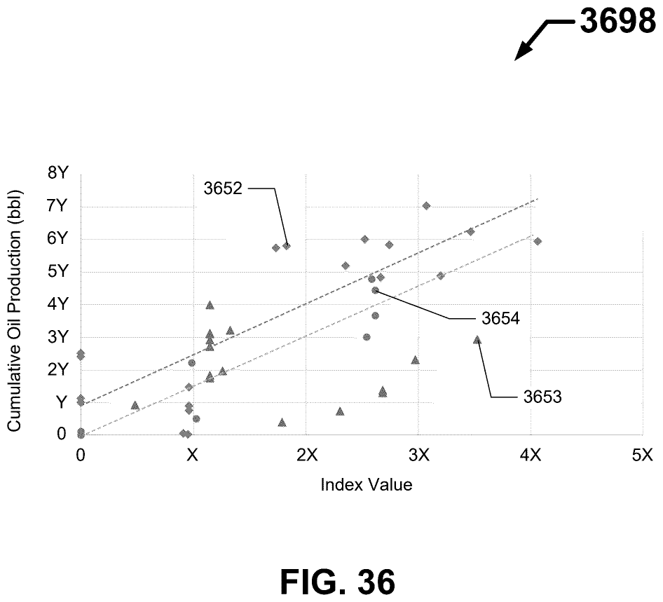

FIG. 36 shows a graph 3698 of actual hydrocarbon production for wells in three different areas based on testing rock samples using the analytic system of FIG. 32 according to certain example embodiments. Referring to the description with respect to FIGS. 1 through 35 above, the graph 3698 of FIG. 36 shows plots of the cumulative production (in bbls) for a wellbore in each area along the vertical axis versus an index quantifying hydrocarbon production potential of the wellbores using the analytic system 3250 of FIG. 32 along the horizontal axis.

Plot 3652 shows the cumulative production versus the measured index value for one wellbore in one area. Plot 3653 shows the cumulative production versus the measured index value for another wellbore in another area. Plot 3654 shows the cumulative production versus the measured index value for yet another wellbore in yet another area. Each area may differ based on one or more of a number of factors, including but not limited to operational characteristics of a wellbore, geological characteristics of the subterranean formation, and geographic location (e.g., distance). The data plotted in the graph 3698 may cover a finite period of time (e.g., one month, 90 days, 6 months, a year).

In some cases, example embodiments may be directed to a method for asset development optimization using a rock sample and rock sample-test fluid interaction testing, where the method may include combining the rock sample and a test fluid for a period of time, where the rock sample originates from a portion of a subterranean formation through which a wellbore is drilled. Such a method may also include obtaining a measurement of a hydrocarbon released from the rock sample-test fluid interaction testing after the period of time, and generating, using the measurement, a forecast of hydrocarbon production potential for a portion of a subterranean formation from which the rock sample is obtained.

In such cases, the hydrocarbon may include a liquid. In addition, or in the alternative, in such cases, the hydrocarbon may include a gas. In addition, or in the alternative, in such cases, the test fluid may include an acid. In such cases, the acid may include at least one of a group consisting of HCl, HNO₃, acetic acid, CO₂, and saturated acidic water. In addition, or in the alternative, in such cases, the test fluid may match a chemical composition of a fracturing fluid. In addition, or in the alternative, in such cases, the test fluid may include a brine. In addition, or in the alternative, in such cases, the rock sample may be among drilling mud circulated to a surface from the wellbore. In addition, or in the alternative, in such cases, the rock sample may include a core sample extracted from the wellbore. In addition, or in the alternative, in such cases, the wellbore at the portion of the subterranean formation may be substantially vertically oriented.

In addition, or in the alternative, in such cases, the measurement may be obtained using gas chromatography. In addition, or in the alternative, in such cases, the measurement may be obtained using an oil in water analyzer. In addition, or in the alternative, in such cases, the measurement may be obtained by measuring a mass of the hydrocarbon. In addition, or in the alternative, in such cases, the measurement may be obtained using water displacement. In addition, or in the alternative, in such cases, the measurement may be obtained using a catalytic bead sensor. In addition, or in the alternative, in such cases, the measurement of the hydrocarbon may include a chemical content of a gas and a volume of the gas.

In some cases, example embodiments may be directed to a system for asset development optimization using a rock sample and rock sample-test fluid interaction testing. In such cases, the system may include a fluid source that is configured to provide a test fluid, an analytic system, and a controller. The analytic system of such a system may include a testing apparatus having a vessel and a sensor device, where the testing apparatus is configured to receive, by the vessel, the rock sample that originates from a portion of a subterranean formation through which a wellbore is drilled; receive, by the vessel, the test fluid from the fluid source; and measure, using the sensor device, a measurement of a hydrocarbon released from the rock sample-test fluid interaction testing in the vessel. The controller of such a system may be configured to facilitate generating, using the measurement, a forecast of hydrocarbon production potential for a portion of a subterranean formation from which the rock sample is obtained.

In such cases, the system may also include a processing system configured to process the rock sample before the rock sample is received by the vessel. In such cases, the processing system may further be configured to process the test fluid before the test fluid is received by the vessel. In addition, or in the alternative, in such cases, the vessel of the testing apparatus may be configured to facilitate the application of a pressure and a temperature to the rock sample and the test fluid.

In some cases, example embodiments may be directed to a computer-implemented method for asset development optimization using a rock sample and rock sample-test fluid interaction testing. In such cases, the computer-implemented method may include facilitate combining the rock sample and a test fluid for a period of time, where the rock sample originates from a portion of a subterranean formation through which a wellbore is drilled. In such cases, the computer-implemented method may also include facilitate obtaining a measurement of a hydrocarbon released from the rock sample-test fluid interaction testing after the period of time. In such cases, the computer-implemented method may further include facilitate generating, using the measurement, a forecast of hydrocarbon production potential for a portion of a subterranean formation from which the rock sample is obtained.

Example embodiments may be used to provide systems and methods for rock-fluid interaction test design to characterize/forecast/improve reservoir and well performance in shale and other TRU plays. Example embodiments may provide a number of benefits. Such benefits may include, but are not limited to, optimizing well performance, optimizing hydraulic fracturing operations, optimizing saltwater disposal operations, optimizing injection and production wells, ease of use, extending the life of a well (including both parent wells and child wells), flexibility, configurability, and compliance with applicable industry standards and regulations.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A method for asset development optimization using a rock sample and rock sample-test fluid interaction testing, the method comprising:
combining the rock sample and a test fluid for a period of time, wherein the rock sample originates from a portion of a subterranean formation through which a wellbore is drilled;
obtaining a measurement of a hydrocarbon released from the rock sample-test fluid interaction testing after the period of time; and
generating, using the measurement, a forecast of hydrocarbon production potential for a portion of a subterranean formation from which the rock sample is obtained.

2. The method of claim 1, further comprising:
determining, using the measurement, a location to place a subsequent wellbore.

3. The method of claim 1, wherein the wellbore at the portion of the subterranean formation is substantially horizontally oriented.

4. The method of claim 3, further comprising:
determining, using the forecast of the hydrocarbon production potential, a production forecast for the wellbore.

5. The method of claim 3, further comprising:
determining, using the forecast of the hydrocarbon production potential, a completion optimization plan for the wellbore.

6. The method of claim 5, wherein the completion optimization plan for the wellbore comprises a chemical composition of a fracturing fluid used in completing the wellbore.

7. The method of claim 3, further comprising:
determining, using the forecast of the hydrocarbon production potential, a location within a layer of the subterranean formation in which to land a substantially horizontal section of the wellbore kicking off from a substantially vertical section of the wellbore.

8. The method of claim 1, wherein the measurement of the hydrocarbon production potential comprises an index value that is based on a baseline.

9. The method of claim 1, wherein generating the forecast of hydrocarbon production potential comprises comparing the measurement to a baseline for the rock sample.

10. The method of claim 1, wherein the period of time is relatively short to determine viability of the wellbore, and wherein the period of time is relatively long to generate a recommendation directed to further development of the wellbore.

11. The method of claim 10, wherein further development of the wellbore comprises at least one of a group consisting of refracturing the wellbore, performing enhanced oil recovery operations on the wellbore, performing simulation treatment operations on the wellbore, and shutting in the wellbore for a period of time.

12. The method of claim 11, wherein performing enhanced oil recovery operations on the wellbore comprises at least one of a group consisting of applying a chemical treatment to the wellbore, applying a surfactant treatment to the wellbore, and applying a polymer treatment to the wellbore.

13. A system for asset development optimization using a rock sample and rock sample-test fluid interaction testing, the system comprising:
a fluid source that is configured to provide a test fluid; and
an analytic system comprising:
a testing apparatus comprising a vessel and a sensor device, wherein the testing apparatus is configured to:
receive, by the vessel, the rock sample that originates from a portion of a subterranean formation through which a wellbore is drilled;
receive, by the vessel, the test fluid from the fluid source; and
measure, using the sensor device, a measurement of a hydrocarbon released from the rock sample-test fluid interaction testing in the vessel; and
a controller communicably coupled to the testing apparatus, wherein the controller is configured to:
facilitate generating, using the measurement, a forecast of hydrocarbon production potential for a portion of a subterranean formation from which the rock sample is obtained.

14. The system of claim 13, wherein the vessel comprises a conical flask.

15. The system of claim 14, wherein the vessel further comprises a stopper disposed at a top end of the conical flask, wherein the stopper has an aperture that traverses therethrough, wherein the aperture has disposed therein a tube through which the test fluid is introduced into an interior of the conical flask.

16. The system of claim 15, wherein the stopper has a second aperture that traverses therethrough, wherein the second aperture has disposed therein a probe for the sensor device.

17. The system of claim 16, wherein the stopper has a third aperture that traverses therethrough, wherein the third aperture has disposed therein an additional tube through which a chemical reagent is introduced into the interior of the conical flask.

18. The system of claim 13, wherein the vessel further comprises cotton wool disposed in a neck of the conical flask.

19. The system of claim 13, further comprising:
a sonication device in communication with the vessel, wherein the sonication device is configured to provide vibrations to the vessel.

20. The system of claim 13, wherein the testing apparatus further comprises a plurality of additional vessels interconnected with the vessel, wherein a first of the plurality of additional vessels is configured to receive the hydrocarbon in gaseous form, and wherein a second of the plurality of additional vessels is configured to receive a fluid forced out of the first of the plurality of additional vessels by the hydrocarbon in gaseous form.
